(19) United States
(12) Patent Application Publication
(10) Pub. No.: US 2026/0199356 A1
(43) Pub. Date: Jul. 16, 2026

(54) Composition and Method to Correct Nutritional Deficiencies underlying Mood Disorder

(71) Applicant: Alfasigma S.p.A., Bologna (IT)

(72) Inventor: Eric R. First, Morristown, NJ (US)

(21) Appl. No.: 19/417,979

(22) Filed: Dec. 12, 2025

Related U.S. Application Data
(60) Provisional application No. 63/733,825, filed on Dec. 13, 2024.

(51) Int. Cl. A61K 31/519; A61K 31/05; A61K 31/121; A61K 31/198; A61K 31/202; A61K 31/385; A61K 31/593; A61K 33/06; A61K 33/30; A61K 36/30; A61K 36/324; A61K 36/53; A61K 36/575; A61K 36/756; A61K 36/81; A61K 36/84; A61K 36/88

(52) U.S. Cl. CPC A61K 31/519; A61K 31/05; A61K 31/121; A61K 31/198; A61K 31/202; A61K 31/385; A61K 31/593; A61K 33/06; A61K 33/30; A61K 36/30; A61K 36/324; A61K 36/53; A61K 36/575; A61K 36/756; A61K 36/81; A61K 36/84; A61K 36/88

(57) ABSTRACT

Disclosed in certain embodiments are compositions and methods for treating, reducing or ameliorating symptoms of a mood disorder and/or re-establishment of homeostasis of nutritional deficiencies.

### FIELD OF THE INVENTION

Disclosed herein are compositions of nutritional formulations, and methods of use thereof. In certain embodiments, the composition and methods of use disclosed herein help in treating, reducing or ameliorating symptoms of a mood disorder and/or re-establishment of homeostasis of nutritional deficiencies.

### BACKGROUND

Over the last 70 years, experts have evolved the hypotheses of the various mechanisms underlying depression and schizophrenia. The monoamine hypothesis of depression was conceived in 1965 and involves imbalances of the monoamine neurotransmitters dopamine, norepinephrine, and serotonin. Since the monoamine hypothesis became the prevailing explanation for select mood and psychiatric disorders in the mid-20th century, therapies Selective Serotonin Reuptake Inhibitors (SSRIs) and Selective Serotonin Norepinephrine Reuptake Inhibitors (SSNRI's), and antipsychotic treatment such as dopamine agonists evolved to target monoamine signaling pathways. The glutamate (or glutamatergic) hypothesis was proposed in the 1990s, when glutamate receptor antagonists were found to have antidepressant-like effects. Since then, research has shown the potential benefit of targeting new treatments such as ketamine towards glutamate neurotransmission in depression, PTSD, Bipolar disorder, and other disorders. More recently, additional hypotheses have emerged, oxidative stress and neuroinflammation and, the involvement of the HPA stress response that leads to chronic anxiety thought to precipitate depression. These new hypotheses are also proposed as new therapeutic targets with many new treatments in development.

There are now many treatments for mood-related disorders, including the use of anti-depressants, anti-psychotics, ketamine, benzodiazepines, anti-epileptics, and various herbs and nutritional ingredients, often used in combination with non-pharmacologic treatments such as cognitive behavioral therapy (CBT), trans-magnetic stimulation (TMS), and electroconvulsive therapy (ECT), among others. Anti-depressants may be helpful to some people to help manage stress and anxiety, but they are specifically intended for depression. Some antidepressants, such as SSRIs, can have adverse effects such as agitation, weight gain, and other side effects. Moreover, their effectiveness in successfully treating depression has been disappointing. As reported in the Sequenced Treatment Alternatives to Relieve Depression (STARD) study, the largest and most comprehensive study to date on the treatment of major depressive disorder (MDD), includes low remission rates, where only one-third of participants achieved remission with the first-line treatment. This results in having to cycle patients on and off various SSRI's, and yet, remission rates decreased with each subsequent treatment cycling step, decreasing to only 13.0% by the fourth step, with only a 67% overall cumulative remission rate across all treatment steps. To address this issue, an emerging treatment approach is the use of adjunctive therapy to augment their response by selecting anti-psychotic medications and or benzodiazepines, which have safety and efficacy issues. Chronic use of anti-psychotics can lead to unwanted movement disorders (tardive dyskinesia), drowsiness, confusion, and weight gain, whereas benzodiazepines can lead to dependence, drowsiness, and foggy thinking. Ketamine, while it is showing promise, does have potential safety issues, including dissociative symptoms and psychotomimetic effects, cardiovascular effect, and chronic and high-frequency use of ketamine has been associated with urological toxicity, including symptoms like dysuria and bladder inflammation, and its potential for abuse.

Nutritional status can contribute to the manifestation of mood disorders. One of the earlier hypotheses of depression was the homocysteine hypothesis, where excess homocysteine can result in deficient folate levels that can contribute to low monoamines and depression. Vitamin D levels have also been shown to be deficient in patients with mood disorders, as well as zinc and other vitamins and minerals. Correcting the underlying nutrient deficiencies has been shown to help improve treatment outcomes. Various amino acidsAshwagandha, such as L-theanine, and herbal supplements, such as saffron and ashwagandha, have been reported to improve mood, anxiety, and depression.

Experts have continued to learn more about the effects of depression, anxiety, and other mood disorders they have on the body. Recently researchers have been looking more deeply into what happens in the brain to cause oxidative stress, glutamate excitotoxicity, and the neuroplastic brain changes in circuitry and function due to oxidative stress, and neuroinflammation which can also damage the brain, just as it can damage other organs and systems. Studies have previously linked chronic depression and anxiety to a higher risk of dementia and Alzheimer's disease.

These recent findings indicate that depression and other mood disorders have multi-factorial pathophysiological processes that contribute to causing problems in the brain, much more than previously thought, and in ways that had not been previously known. While more research is needed to determine what new treatments can address these processes, one thing is clear: treatments over the years have focused largely on one of these main pathophysiologic pathways and what is needed are new compositions and methods to more effectively target multiple pathways to enhance effectiveness in ameliorating depression and other mood related disorders.

There continues to be a need for compositions and methods for the treatment of mood disorders.

### OBJECTS AND SUMMARY

Certain embodiments of the present invention are directed to a pharmaceutical composition comprising a therapeutically effective amount of a compound to treat neurotransmitter signaling disturbance and a therapeutically effective amount of a compound to treat stress response.

In certain embodiments, the pharmaceutical composition further comprises a therapeutically effective amount of a compound to treat glutamate over reactivity.

In certain embodiments, the pharmaceutical composition further comprises a therapeutically effective amount of a compound to treat oxidative stress.

In certain embodiments, the pharmaceutical composition further comprises a therapeutically effective amount of a compound to treat neuroinflammation.

In certain embodiments, the pharmaceutical composition further comprises a therapeutically effective amount of a compound to treat neural communication and plasticity.

In certain embodiments, the present invention is directed to a method of treating mood disorders comprising administering to a patient in need thereof a therapeutically effective amount of a compound to treat neurotransmitter signaling disturbance and a therapeutically effective amount of a compound to treat stress response, wherein the compounds are in the same or different pharmaceutical compositions.

In certain embodiment, the method further comprises a therapeutically effective amount of a compound to treat glutamate overreactivity wherein the compound is in the same or different compositions.

In certain embodiments, the method further comprises a therapeutically effective amount of a compound to treat oxidative stress, wherein the compound is in the same or different compositions.

In certain embodiments, the method further comprises a therapeutically effective amount of a compound to treat neuroinflammation, wherein the compound is in the same or different compositions.

In certain embodiments, the method further comprises a therapeutically effective amount of a compound to treat neural communication and plasticity, wherein the compound is in the same or different compositions.

In certain embodiments, the present invention is directed to a method of treating mood disorders comprising administering to a patient in need thereof a pharmaceutical composition as disclosed herein.

In certain embodiments, the present invention is directed to a kit comprising a therapeutically effective amount of a compound to treat neurotransmitter signaling disturbance and a therapeutically effective amount of a compound to treat stress response, wherein the compounds are in different pharmaceutical compositions.

Certain kit embodiments further comprise a therapeutically effective amount of a compound to treat glutamate overactivity, wherein the compound is in the same or a different composition.

Certain kit embodiments further comprise a therapeutically effective amount of a compound to treat oxidative stress, wherein the compound is in the same or a different composition.

Certain kit embodiments further comprise a therapeutically effective amount of a compound to treat neuroinflammation, wherein the compound is in the same or a different composition.

Certain kit embodiments further comprise a therapeutically effective amount of a compound to treat neural communication and plasticity, wherein the compound is in the same or a different composition.

In certain embodiments, the compound to treat neurotransmitter signaling disturbance is selected from folic acid, folinic acid, 5-methyltetrahydrofolate (D- and/or L-isomers; 5MTH), Calcium L-Methylfolate (LMF) Sodium L-methylfolate, Glucosamine L-methylfolate, vitamin B12 (methylcobalamin), Vitamin B12 (cyanocobalamin), magnesium, 5-hydroxytryptophan (5-HTP), S-adenosylmethionine (SAMe); amino acids Acetyl-L-carnitine (ALC or ALCAR), L-Carnitine (Levocarnitine), Propionyl-L-Carnitine (PLC), Isovaleryl-L-Carnitine, alpha-lipoic acid, N-acetylcysteine, and L-tryptophan, Zinc gluconate, Zinc Picolinate, Zinc Citrate, Zinc Acetate, Zinc Sulfate, Zinc Methionine (Zinc Monomethionine), Zinc Orotate, Zinc Oxide; Iron, Magnesium Oxide (MgO), Magnesium Picolinate (MgPic), Magnesium Citrate, Magnesium L-Threonate (MgLT), Magnesium Glycinate (Bisglycinate), Magnesium Sulfate (MgSO₄); St. John's Wort, Oregano extract, Curcumin, Ginseng (Panax ginseng), Rhodiola rosea, Saffron (Crocus sativus), Luteolin, Tilianin (from Agastache mexicana), L-theanine, pharmaceutically acceptable salts thereof and combinations thereof.

In certain embodiments, the compound to treat neurotransmitter signaling disturbance is L-methylfolate or a pharmaceutically acceptable salt thereof.

In certain embodiments, the compound to treat stress response is selected from L-theanine, valerian root, lemon balm, passionflower, magnolia extract, phaelladendron extract, saffron extract, ashwagandha, 1-methylfolate, folinic acid, vitamin D3, Zinc, pharmaceutically acceptable salts thereof and combinations thereof.

In certain embodiments, the compound to treat stress response is selected from vitamin D3, L-theanine, pharmaceutically acceptable salts thereof, and combinations thereof.

In certain embodiments, the compound to treat glutamate overreactivity is selected from folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Magnesium Picolinate (MgPic), Magnesium Citrate, Magnesium L-Threonate (MgLT), Magnesium Glycinate (Bisglycinate), Magnesium Sulfate (MgSO4); gamma amino butyric acid (GABA), L-theanine and combinations thereof.

In certain embodiments, the compound to treat glutamate overactivity is selected from L-methylfolate, vitamin D3, pharmaceutically acceptable salts thereof, and combinations thereof.

In certain embodiments, the compound to treat oxidative stress is selected from folic acid, Folinic acid, 5-methyltetrahydrofolate (D- and/or L-isomers; 5MTH), Calcium L-Methylfolate (LMF) Sodium L-methylfolate, Glucosamine L-methylfolate, Acetyl-L-carnitine (ALC or ALCAR), L-Carnitine (Levocarnitine), Propionyl-L-Carnitine (PLC), Isovaleryl-L-Carnitine, glutathione, coenzyme Q10, N-acetylcysteine, inositol, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Apple phenolic extracts, Cinnamic acid, Curcumin, Flower essential oil of Tagetes minuta, p-Coumaric acid, Tilapia skin peptides, Flower essential oil of Tagetes minuta, Melatonin, Carvedilol, Saffron extract, Luteolin-7-O-glucuronide, Rosmarinic acid, Celastrol, Thymoquinone, L-theanine and combinations thereof.

In certain embodiments, the compound to treat oxidative stress is selected from 1-methylfolate, vitamin D3, L-theanine, zinc, pharmaceutically acceptable salts thereof, and combinations thereof.

In certain embodiments, the compound to treat neuroinflammation is selected from Calcium L-Methylfolate (LMF) Sodium L-methylfolate, vitamin D3, Zinc, Omega-3 fatty acids (DHA/EPA), Ahi flower oil, alpha-lipoic acid (ALA), medium chain triglyceride (MCT) oil, Curcumin, Boswellia, Resveratrol, L-theanine, and combinations thereof.

In certain embodiments, the compound to treat neuroinflammation is selected from 1-methylfolate, vitamin D3, pharmaceutically acceptable salts thereof and combinations thereof.

In certain embodiments, the compound to treat neural communication and plasticity is selected from folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide; Magnesium Picolinate (MgPic), Magnesium Citrate, Magnesium, L-Threonate (MgLT), Magnesium Glycinate (Bisglycinate), Magnesium Sulfate (MgSO4); Vitamin D3, Curcumin, Ginseng (Panax ginseng) Bacopa monniera; Flavonoids extracts found in various fruits, vegetables, and teas, including, polyphenols Persimmon leaf extract, and Curcumin.

In certain embodiments, the compound to treat neural communication and plasticity is selected from 1-methylfolate, zinc, pharmaceutically acceptable salts thereof and mixtures thereof.

In certain embodiments, the pharmaceutical composition comprises a therapeutically effective amount of a compound to treat neurotransmitter signaling disturbance, a therapeutically effective amount of a compound to treat stress response, a therapeutically effective amount of a compound to treat glutamate overreactivity, a therapeutically effective amount of a compound to treat oxidative stress, a therapeutically effective amount of a compound to treat neuroinflammation and a therapeutically effective amount of a compound to treat neural communication and plasticity.

In certain embodiments, the therapeutically effective amount of a compound to treat neurotransmitter signaling disturbance comprises 1-methylfolate, the therapeutically effective amount of a compound to treat stress response comprises vitamin D3 and L-theanine, the therapeutically effective amount of a compound to treat glutamate overactivity comprises 1-methylfolate and vitamin D3, the therapeutically effective amount of a compound to treat oxidative stress comprises 1-methylfolate, vitamin D3, L-theanine and zinc, the therapeutically effective amount of a compound to treat neuroinflammation comprises 1-methylfolate and vitamin D3 and the therapeutically effective amount of a compound to treat neural communication and plasticity comprises 1-methylfolate and zinc.

In certain embodiments, the pharmaceutical composition is selected from a dosage form for oral, parenteral, subcutaneous, intramuscular, intraperitoneal, intravenous, transdermal, transmucosal, transurethral, rectal, sublingual, buccal, gingival, rectal, subcutaneous, transpulmonary, topical, nasal, or inhalation administration. In a particular embodiment, the composition is an oral dosage form, e.g., a tablet, capsule, softgel, liquid, suspension, solution or powder.

The recitation of an active agent herein is meant to include all pharmaceutically acceptable salts thereof, regardless whether it is specifically indicated.

In certain embodiment, the oral dosage form comprises from about 1 mg to about 75 mg 1-methylfolate, from about 1,000 IU to about 100,000 IU vitamin D3, from about 25 mg to about 500 mg L-theanine, and from about 15 mg to about 250 mg zinc.

In certain embodiment, the oral dosage form comprises from about 10 mg to about 20 mg 1-methylfolate, from about 1,000 IU to about 3,000 IU vitamin D3, from about 200 mg to about 300 mg L-theanine, and from about 10 mg to about 40 mg zinc.

In certain embodiment, the oral dosage form comprises about 15 mg 1-methylfolate, about 2,000 IU vitamin D3, about 250 mg L-theanine, and about 25 mg zinc.

In certain embodiment, the oral dosage form comprises from about 1 mg to about 50 mg 1-methylfolate, from about 1,000 IU to about 100,000 IU vitamin D3, from about 1 mg to about 50 mg folinic acid, from about 1 mg to about 100 mg vitamin B6, from about 1 mg to about 500 mg magnesium and from about 5 mg to about 100 mg saffron.

In certain embodiments, the oral dosage form comprises from about 1 mg to about 50 mg 1-methylfolate, from about 1,000 IU to about 100,000 IU vitamin D3, from about 1 mg to about 50 mg folinic acid, from about 1 mg to about 500 mg iron, from about 100 mg to about 1,000 mg glutathione and from about 5 mg to about 100 mg saffron.

In certain embodiment, the oral dosage form comprises from about 1 mg to about 50 mg 1-methylfolate, from about 1,000 IU to about 100,000 IU vitamin D3, from about 250 mg to about 3,000 mg acetyl-1-carnitine, from about 1 mg to about 500 mg zinc and from about 25 mg to about 500 mg L-theanine.

In certain embodiments, the present invention is directed to a method of treating a mood disorder, e.g., selected from Major Depressive Disorder, Persistent Depressive Disorder, Bipolar Depression, Seasonal Affective Disorder, Atypical Depression, Anhedonia, General Anxiety Disorder, ADHD, ADD, variants thereof or combinations thereof.

In certain embodiments, the administration is adjunctive therapy to a different treatment.

In certain embodiments, the administration is adjunctive therapy to treatment with an SSRI, SNRI, typical antipsychotic, atypical antipsychotic, dopamine agonist, ketamine, or a derivative thereof (e.g., s-ketamine), psychedelic, MDMA, psylocybin, LSD or a combination thereof.

In other embodiments, the administration is adjunctive therapy to Cognitive Behavioral Therapy, transmagnetic stimulation, electroconvulsive treatment, or a combination thereof.

In certain embodiments, the administration is initial therapy.

In certain embodiments, the administration is after discontinuation of a different treatment.

In certain embodiments, the administration is OD, BID, TID or QID.

In certain kit embodiments, the present invention further comprises instructions for treating a mood disorder selected from Major Depressive Disorder, Persistent Depressive Disorder, Bipolar Depression, Seasonal Affective Disorder, Atypical Depression, Anhedonia, General Anxiety Disorder, ADHD, ADD, variants thereof or combinations thereof.

Certain kit embodiments, further comprise instruction for administration as adjunctive therapy to a different treatment, e.g., treatment with an SSRI, SNRI, typical antipsychotic, atypical antipsychotic, dopamine agonist, ketamine or a derivative thereof (e.g., s-ketamine), psychedelic, MDMA, psylocybin, LSD or a combination thereof.

Certain kit embodiments further comprise instruction for administration that is initial therapy, administration after discontinuation of a different treatment and/or administration that is OD, BID, TID or QID.

Certain embodiments are directed to a method of administration to treat the nutritional requirements for neurotransmitter imbalances in the clinical dietary management of mood disorders, comprising administering a pharmaceutical composition of any preceding claim.

Certain embodiments are directed to a method of administration to treat the nutritional requirements for neurotransmitter imbalances in the clinical dietary management of mood disorders, comprising administering a pharmaceutical composition of a kit of any preceding claim.

As used herein, disclosure of an active agent according to embodiments of the present invention is meant to include all pharmaceutically acceptable salts and derivatives thereof. For example, recitation of 1-methyl folate is meant to encompass 1-methylfolate calcium, recitation of vitamin D3 is meant to encompass cholecalciferol, recitation of zinc is meant to encompass zinc bisglycinate and recitation of L-theanine is meant to encompass y-glutamylethylamide.

### BRIEF DESCRIPTION OF THE DRAWINGS

The figures are not necessarily to scale, emphasis instead generally being placed upon illustrative principles. The figures are to be considered illustrative in all aspects and are not intended to limit the disclosure, the scope of which is defined only by the claims.

FIG. 1 illustratively depicts a flowchart of the pathophysiology of nutrient deficiencies within six main process pathways involved in mood regulation.

FIG. 2 illustratively depicts a flowchart of the combination of target and effects from four exemplary ingredients in the dosage form according to embodiments herein.

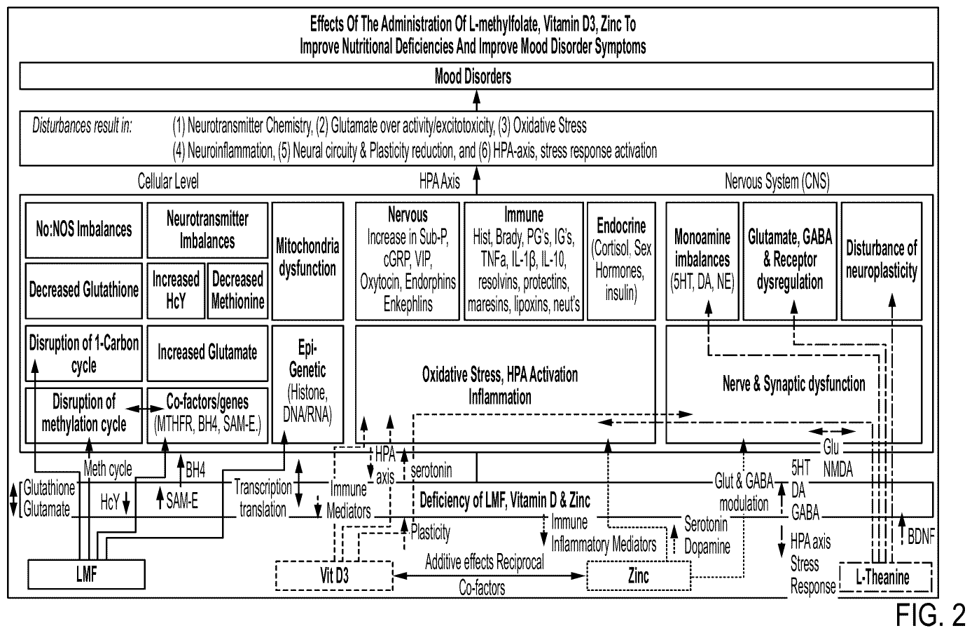

### DETAILED DESCRIPTION

According to various embodiments, disclosed herein is a composition of nutritional ingredients along with pharmaceutically acceptable excipients that are selected based on their targeted effects on one or more of six key process pathways that mediate mood disorders and have underlying nutritional deficiencies as a potential root cause, and any related process pathways thought to underlie the pathogenesis of depression and related mood disorders including; (1) neurotransmitter and receptor synthesis, including the monoamines (5-HT, DA, NE) and release, (2) glutamate and NMDA receptor and GABA modulation, (3) oxidative stress modulation, (4) neuroinflammation, (5) neural communication and plasticity, (6) Hypothamic-Pituitary_Adrenal Axis (HPA) stress response modulation, with the resulting, all-inclusive targeting of these six major process pathways and restoration of nutrient deficiencies to maintain allostasis across these pathways. In certain embodiments, the disclosed compositions may have additive and or synergistic effects within at least one of the six process pathways, and administration of these compositions, address the mood symptoms acutely and restores these nutrient deficiencies to maintain allostasis in the long term.

### Overview of the Six Processes and Pathways Affected as a Result of a Mood Disorder

According to various embodiments, is a disclosure of a composition of ingredients along with optional pharmaceutically acceptable excipients that are selected based on their targeted effects on one or more of six key process pathways that underlie the pathogenesis of mood disorders such as depression and schizophrenia. For the (1) neurotransmitter and receptor synthesis process, known as a “Neurotransmitter Modulator” ingredients can be selected for beneficial effects within for epigenetic histone activation, one-carbon and methylation cycles, homocysteine to methionine conversion, monoamines (5HT, DA, NE) and related neurotransmitters for their synthesis, production, release, inhibition, reuptake and; steroids, hormones, enzymes and cofactors including but not limited to the following processes; dihydrobiopterin to tetrahydrobipterin (BH2: BH4); methionine to S-adenosyl-S-adenosylmethionine (MET: SAMe), nitric oxide synthase to nitric oxide (NOS: NO), homocysteine to methionine (HcY: MET); mediators involved in the Monoamine Synthesis Pathways including, Tyrosine Hydroxylase (TH) which converts tyrosine to L-DOPA, the precursor of dopamine; Tryptophan Hydroxylase (TPH), converts tryptophan to 5-hydroxytryptophan (5-HTP), precursor of serotonin; Enzymes: Monoamine Oxidase (MAO): MAO-A and MAO-B involved in the oxidative deamination of monoamines dopamine,, serotonin, and norepinephrine, producing hydrogen peroxide as a byproduct, which can contribute to oxidative stress; Catechol-O-Methyltransferase (COMT), degrades catecholamines like dopamine, norepinephrine, and epinephrine by methylation; Cofactors and Vitamins: Vitamin B6 (Pyridoxal Phosphate), acts as a cofactor for enzyme aromatic L-amino acid decarboxylase (AADC), which converts L-DOPA to dopamine and 5-HTP to serotonin; Folate and L-Methylfolate: essential for the synthesis of SAMe, which is involved in methylation processes critical for neurotransmitter synthesis and metabolism; Minerals: Iron, required as a cofactor for tyrosine hydroxylase and tryptophan hydroxylase; Zinc, modulates neurotransmitter receptors and transporters, influencing synaptic plasticity and neurotransmission; Transporters: Monoamine Transporters (DAT, NET, SERT) responsible for the reuptake of dopamine, norepinephrine, and serotonin, respectively, from synaptic cleft back into presynaptic neurons, thus regulating their availability and signaling; Regulatory Pathways: CAMP Response Element-Binding Protein (CREB), a transcription factor regulating expression of genes involved in neurotransmitter synthesis and plasticity; Protein Kinases: Such as PKA, PKC, and CaMKII, which modulate activity and trafficking of monoamine transporters and receptors

For the (2) glutamate and GABA modulation, a “Glutamate modulator” can be selected from ingredients that have a beneficial effects within for example, at down-regulating the levels of glutamate excitatory level and upregulating GABA inhibitory levels selected from one or more of the following; Glutamate-Glutamine Cycle involved conversion of glutamate to glutamine in astrocytes and its subsequent conversion back to glutamate in neurons, which is essential for maintaining glutamate homeostasis and involves enzymes such as glutamine synthetase and phosphate-activated glutaminase; NMDA Receptor Regulation, the receptors are modulated by various endogenous agents and signaling pathways and its key regulators include: G-Protein-Coupled Receptors (GPCRs) which modulate NMDA receptor activity via intracellular signaling pathways, affecting receptor expression, localization, and function, Posttranslational Modifications via Phosphorylation, ubiquitination, and palmitoylation of NMDA receptors influence their stability, trafficking, and synaptic expression; GABA Synthesis and Recycling, GABA is synthesized from glutamate by the enzyme glutamate decarboxylase (GAD) and the GABA-glutamine cycle, similar to the glutamate-glutamine cycle, involves the recycling of GABA between neurons and astrocytes; Vitamin B6 a cofactor for GAD, facilitates conversion of glutamate to GABA; Magnesium which modulates NMDA receptor activity by acting as a natural blocker of the receptor's ion channel, preventing excessive calcium influx and excitotoxicity; Zinc Modulation of both glutamate and GABA receptors, influencing synaptic plasticity and neurotransmission; and Antioxidant Systems: Glutathione, a major antioxidant, protects neurons from oxidative stress, where its synthesis involves enzymes such as glutamate-cysteine ligase (GCLC) and glutathione synthetase, among other mechanisms affected by nutritional deficiencies.

For the (3) oxidative stress modulation, known as a “Oxidative Stress Modulator” ingredients can be selected that have beneficial effects within for example; Reactive Oxygen Species (ROS) and Reactive Nitrogen Species (RNS), including superoxide anion (O₂⁻), hydrogen peroxide (H₂O₂), hydroxyl radicals (·OH), nitric oxide (NO), and peroxynitrite (ONOO⁻) that are central to oxidative stress and produced by various cellular processes, including mitochondrial respiration and NADPH oxidase activity; NADPH Oxidases (NOX), a family of enzymes, including NOX1, NOX2, NOX3, NOX4, NOX5, DUOX1, and DUOX2, which are primary sources of ROS in the CNS which transfer electrons from NADPH to oxygen, generating superoxide anions; Antioxidant Defense Systems including key antioxidants, superoxide dismutase (SOD), catalase, and glutathione peroxidase (GPx), that neutralize ROS, converting them into less harmful molecules for example, SOD conversion of superoxide anions into hydrogen peroxide, which is then broken down by catalase and GPx; Nuclear Factor Erythroid 2-Related Factor 2 (Nrf2), a transcription factor regulating expression of antioxidant proteins and when activated, Nrf2 translocates to nucleus and binds to antioxidant response element (ARE), promoting expression of genes involved in detoxification and antioxidant defense, such as glutathione S-transferase and NAD (P) H: quinone oxidoreductase 1 (NQO1); Glutathione (GSH), a major intracellular antioxidant neutralizes ROS and RNS and is synthesized from glutamate, cysteine, and glycine, with glutamate-cysteine ligase (GCLC) being the rate-limiting enzyme in its synthesis, GSH also participates in detoxification of harmful substances through conjugation reactions; Vitamin E, a lipid-soluble antioxidant protects cell membranes from lipid peroxidation; Vitamin C, a water-soluble antioxidant regenerates vitamin E and neutralizes ROS; Zinc and Selenium, essential cofactors for antioxidant enzymes like SOD and GPx, respectively, among other mechanisms affected by nutritional deficiencies.

For (4) neuroinflammation, ingredients can be selected that have beneficial effects within for example; Pro-inflammatory cytokines such as tumor necrosis factor-alpha (TNF-α), interleukin-1 beta (IL-1B), and interleukin-6 (IL-6), critical mediators released in response to microglial and astrocyte activation and release of releasing cytokines and chemokines, Toll-Like Receptors (TLRs): TLRs, particularly TLR4, involved in recognizing pathogen-associated molecular patterns (PAMPs) and damage-associated molecular patterns (DAMPs), Activation of TLRs and activation of downstream signaling pathways, including MyD88 and TRAF6, which promote the production of pro-inflammatory cytokines, Transcription Factor, Nuclear Factor kappa B (NF-κB) that regulates expression of various inflammatory genes, and its activation of NF-κB in glial cells leading to the production of cytokines, chemokines, and other inflammatory mediators; Inflammasomes: such as the NLRP3, multiprotein complexes that detect cellular stress and activate caspase-1, leading to cleavage and release of pro-inflammatory cytokines IL-1β and IL-18, Reactive Oxygen Species (ROS) generated during neuroinflammation exacerbating neuronal damage; Antioxidant systems, including glutathione, which are crucial in mitigating ROS-induced damage; Vitamins and Minerals such as vitamin D and zinc and magnesium which modulate neuroinflammation, whereas, Vitamin D for instance, has anti-inflammatory properties influencing expression of cytokines and other inflammatory mediators; Enzymes such as cyclooxygenase-2 (COX-2) and inducible nitric oxide synthase (iNOS) upregulated during neuroinflammation and contribute to the production of pro-inflammatory mediators, among other mechanisms affected by nutritional deficiencies.

For (5) Neurotrophic and Synaptic Plasticity, a “Neurotrophic Modulator” can be selected that have beneficial effects within for example; Brain-Derived Neurotrophic Factor (BDNF), a critical mediator of neuroplasticity promoting formation, stabilization, and potentiation of synapses through its high-affinity TrkB receptors; Mitogen-Activated Protein Kinase/Extracellular Signal-Regulated Kinase (MAPK/ERK) Pathway, essential for synaptic plasticity and long-term potentiation (LTP), activation of ERK leads to expression of plasticity-related proteins such as c-Fos, Egr-1, and Arc; cAMP Response Element-Binding Protein (CREB), a transcription factor that, when phosphorylated, promotes expression of genes involved in neuroplasticity, including BDNF; Nitric Oxide (NO) synthesized by neuronal nitric oxide synthase (nNOS) contributing to neuroplasticity by activating the cGMP/PKG pathway, which in turn activates ERK signaling; Glutamate, the primary excitatory neurotransmitter has a crucial role in synaptic plasticity, and activation of its NMDA receptors, which are essential for LTP and synaptic strengthening; GABA, primary inhibitory neurotransmitter, helps balance excitatory signals (GLUT: GABA) and is involved in synaptic plasticity by modulating neuronal excitability (GLUT); Vitamin D modulates neuroplasticity by influencing expression of neurotrophic factors and reducing inflammation; Zinc, essential for synaptic plasticity, modulates neurotransmitter release and receptor function; Magnesium, Involved in NMDA receptor function and synaptic plasticity.

For (6) HPA stress response modulation, a “HPA Modulator” can be selected that have beneficial effects within for corticotropin-releasing hormone to adrenocorticotropic hormone (CRH: ACTH), glucocorticoids including cortisol (ACTH: CORT), Glucocorticoid Receptors (CORT: GRs), Mineralocorticoid Receptors (MRs), Arginine Vasopressin (AVP), activation of the sympatho-adrenomedullary (SAM) system and release of Catecholamines in particular; epinephrine (EPI) and norepinephrine (NE), also known as, adrenaline and noradrenaline and their modulation of CRH release, immune system activation and release of Inflammatory cytokines in particular; pro-inflammatory cytokines such as IL-1β, IL-6, and TNF-α which can stimulate HPA axis by increasing CRH and ACTH release, Vitamin D modulation of the HPA axis by influencing expression of enzymes involved in glucocorticoid metabolism and by exerting anti-inflammatory effects.

According to certain embodiments disclosed herein, disclosed is a composition of ingredients along with pharmaceutically acceptable excipients that are selected based on their targeted effects on one or more of the six key pathways already disclosed resulting in an all-inclusive targeting of up to six process pathways. In certain embodiments, the ingredients have an additive or synergistic effect within processes for an attenuated overall response leading to 1) acute improvement in symptoms and 2) long-term correction and balance of nutritional deficiencies involved in these process pathways.

Ingredients with Effects Within The Six Process Pathways

According to certain embodiments, disclosed is a composition of ingredients along with optional pharmaceutically acceptable excipients that are selected based on their targeted effects on one or more and preferably all of the six key process pathways previously disclosed. Certain embodiments have an additive or synergistic effects among ingredients and within processes for an attenuated overall response leading to 1) acute improvement in symptoms and 2) long term correction and balance of nutritional deficiencies involved in these process pathways. In certain embodiments, each ingredient is involved in 2 or more process pathways, and most preferably, each ingredient is involved in all six process pathways.

### Neurotransmitter (Monoamine) Modulators

Further embodiments include ingredients that are selected from one or more of the following non-limiting examples of ingredients: folic acid, folinic acid, 5-methyltetrahydrofolate (D- and/or L-isomers; 5MTH), Calcium L-Methylfolate (LMF) Sodium L-methylfolate, Glucosamine L-methylfolate, vitamin B12 (methylcobalamin), Vitamin B12 (cyanocobalamin), magnesium, 5-hydroxytryptophan (5-HTP), S-adenosylmethionine (SAMe); amino acids Acetyl-L-carnitine (ALC or ALCAR), L-Carnitine (Levocarnitine), Propionyl-L-Carnitine (PLC), Isovaleryl-L-Carnitine, alpha-lipoic acid, N-acetylcysteine, and L-tryptophan, Zinc gluconate, Zinc Picolinate, Zinc Citrate, Zinc Acetate, Zinc Sulfate, Zinc Methionine (Zinc Monomethionine), Zinc Orotate, Zinc Oxide; Iron, Magnesium Oxide (MgO), Magnesium Picolinate (MgPic), Magnesium Citrate, Magnesium L-Threonate (MgLT), Magnesium Glycinate (Bisglycinate), Magnesium Sulfate (MgSO4); St. John's Wort, Oregano extract, Curcumin, Ginseng (Panax ginseng), Rhodiola rosea, Saffron (Crocus sativus), Luteolin, Tilianin (from Agastache mexicana) and L-theanine.

In certain embodiments, the ingredient is selected from one or more of the following non-limiting examples; Calcium L-Methylfolate, folinic acid, Calcium L-Methylfolate (LMF) Sodium L-methylfolate, vitamin B12 (methylcobalamin), Vitamin B6 (Pyridoxal Phosphate); Vitamin D3; Zinc Bisglycinate, Zinc Citrate, Iron, Magnesium, St. John's Wort, Saffron (Crocus sativus), and L-theanine.

In certain embodiments, the composition is comprised of Calcium L-Methylfolate, Vitamin D3; Zinc Bisglycinate and L-theanine.

### Oxidative Stress Modulators

Further embodiments include ingredients that are selected from one or more of the following non-limiting examples, folic acid, folinic acid, 5-methyltetrahydrofolate (D- and/or L-isomers; 5MTH), Calcium L-Methylfolate (LMF) Sodium L-methylfolate, Glucosamine L-methylfolate, Acetyl-L-carnitine (ALC or ALCAR), L-Carnitine (Levocarnitine), Propionyl-L-Carnitine (PLC), Isovaleryl-L-Carnitine, glutathione, coenzyme Q10, N-acetylcysteine, inositol, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Apple phenolic extracts, Cinnamic acid, Curcumin, Flower essential oil of Tagetes minuta, p-Coumaric acid, Tilapia skin peptides, Flower essential oil of Tagetes minuta, Melatonin, Carvedilol, Saffron extract, Luteolin-7-O-glucuronide, Rosmarinic acid, Celastrol, Thymoquinone and L-theanine.

In certain embodiments, the ingredients are selected from one or more of the following non-limiting examples; folinic acid, Calcium L-Methylfolate (LMF) Sodium L-methylfolate, Acetyl-L-carnitine (ALC or ALCAR), L-Carnitine (Levocarnitine), glutathione, coenzyme Q10, N-acetylcysteine, inositol, Zinc Bisglycinate, Zinc Oxide, Curcumin, Saffron extract, and L-theanine.

In certain embodiments, the composition is comprised of Calcium L-Methylfolate, Vitamin D3; Zinc Bisglycinate and L-theanine.

### Anti-Inflammatory Modulators

Further embodiments include ingredients that are selected from one or more of the following non-limiting examples, Calcium L-Methylfolate (LMF) Sodium L-methylfolate, vitamin D3, Zinc, Omega-3 fatty acids (DHA/EPA), Ahi flower oil, alpha-lipoic acid (ALA), medium chain triglyceride (MCT) oil, Curcumin, Boswellia, Resveratrol and L-theanine.

In certain embodiments, the ingredients are selected from one or more of the following non-limiting examples; Calcium L-Methylfolate (LMF) Sodium L-methylfolate, Zinc Bisglycinate, Zinc oxide, Vitamin D3, and L-theanine;

In certain embodiments, the composition is comprised of, Calcium L-Methylfolate (LMF), Zinc Bisglycinate, Vitamin D3 and L-theanine.

### Glutamate-GABA Modulators

Further embodiments include ingredients that are selected from one or more of the following non-limiting examples; folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Magnesium Picolinate (MgPic), Magnesium Citrate, Magnesium L-Threonate (MgLT), Magnesium Glycinate (Bisglycinate), Magnesium Sulfate (MgSO₄); gamma amino butyric acid (GABA), and L-theanine.

In certain embodiments, the ingredients are selected from one or more of the following non-limiting examples; folinic acid, Calcium L-Methylfolate (LMF) Sodium L-methylfolate, Vitamin D3, Zinc Citrate, Zinc Bisglycinate, Magnesium L-Threonate (MgLT), Magnesium Glycinate (Bisglycinate); gamma amino butyric acid (GABA), L-theanine;

In certain embodiments, the composition is comprised of; Calcium L-Methylfolate (LMF) Sodium L-methylfolate; Vitamin D3, Zinc Bisglycinate, and L-theanine.

### Neurotrophic and Synaptic Plasticity Modulators

Further embodiments include ingredients that are selected from one or more of the following non-limiting examples; folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide; Magnesium Picolinate (MgPic), Magnesium Citrate, Magnesium, L-Threonate (MgLT), Magnesium Glycinate (Bisglycinate), Magnesium Sulfate (MgSO₄); Vitamin D3, Curcumin, Ginseng (Panax ginseng) Bacopa monniera; Flavonoids extracts found in various fruits, vegetables, and teas, including, polyphenols Persimmon leaf extract, and Curcumin.

In certain embodiment, the ingredients are selected from one or more of the following non-limiting examples; folic acid, folinic acid, Calcium L-Methylfolate (LMF) Sodium L-methylfolate; Vitamin D3, Zinc Bisglycinate, Zinc Citrate and L-theanine; and Curcumin.

In certain embodiments, the composition is comprised of; Calcium L-Methylfolate (LMF) Sodium L-methylfolate; Vitamin D3, Zinc Bisglycinate, and L-theanine;

### HPA Stress and Anxiety Modulators:

Non-limiting examples of ingredients known to have an effect on the HPA stress response and anxiety: L-theanine, Valerian root, Lemon balm, Passionflower, Magnolia extract, Phaelladendron extract, Saffron extract, and Ashwagandha, 1-methylfolate, Folinic acid, vitamin D3, and Zinc. Preferably, the composition is comprised of, L-theanine which modulates the HPA axis, lowering serum cortisol and glutamate levels to reduce chronic stress and its impact on neurons, and Vitamin D3 known to mitigate HPA axis-induced inflammation by regulating the immune system and pro-inflammatory cytokines.

In certain embodiments, ingredients are selected from one or more of the following non-limiting examples; folic acid, Folinic acid, Calcium L-Methylfolate (LMF) Sodium L-methylfolate; Vitamin D3, Zinc Bisglycinate, Zinc Citrate and L-theanine;

In certain embodiments, the composition is comprised of; Calcium L-Methylfolate (LMF) Sodium L-methylfolate; Vitamin D3, Zinc Bisglycinate, and L-theanine;

In certain embodiments, vitamins, mineral, amino acids, herbs, extracts, and botanical ingredients that have an effect on one or more of the six pathways, providing an enhanced overall response. These nutrients work through various mechanisms, including modulation of neurotransmitter synthesis, reduction of inflammation, and enhancement of neuroplasticity, thereby contributing to the alleviation of depressive symptoms.

### Pharmaceutical Drugs

In certain embodiments, dosage forms as disclosed herein include at least one additional active ingredient. The, at least one additional active ingredient, can include at least one of a mood disorder drug, an antidepressant, an anti-psychotic, anti-anxiety, autism, attention deficit, a cannabinoid, dronabinol, a prodrug thereof, a pharmaceutically acceptable salt thereof and combinations thereof. In certain methods, the additional active agent can be in a separate dosage form suitable for any route of administration.

Certain embodiments disclosed herein are directed to a composition of ingredients along with pharmaceutically acceptable excipients that are selected based on their targeted effects on one or more of 6 key pathways already disclosed that underlie the pathogenesis of mood disorders such as depression.

### Dosage Forms and Preparation

According to certain embodiments, the compositions disclosed herein can be prepared by methods that include, but are not limited to dry blending, wet granulation, dry granulation, compression, extrusion, encapsulation, and any other suitable methods including gummies, topical formulations, liquids, etc.

### Nutritional Ingredient Modulators

According to certain embodiments disclosed herein, suitable dosage forms comprise one or more of a nutritional ingredient selected from one or more of 1. Neurotransmitter (monoamine) modulators, 2. Glutamate-GABA modulators, 3. Oxidative Stress modulators, 4. Neuroinflammation modulators, 5. Neurotrophic/synaptic plasticity modulators, and 6. HPA stress response modulators, and combinations thereof.

In certain embodiments disclosed herein, one active agent can be used for more than one pathway. In other embodiments, one or more active agents can be used for each pathway.

### Pharmaceutical Excipients

According to certain embodiments, one or more ingredients of the dosage forms can be prepared in a matrix in the form of particles, a powder, granules, beads, microspheres and combinations thereof. Suitable diluents include, but are not limited to, lactose, microcrystalline cellulose, starch, powdered sucrose, mannitol, fructose, sorbitol, calcium phosphate, and calcium sulphate. Suitable granulating agents are solutions of povidone, an aqueous preparation of cornstarch, molasses, methylcellulose, hydroxypropyl methylcellulose, carboxymethylcellulose, glucose solution, and microcrystalline cellulose. Suitable dry binders or nonaqueous solution can be used for substances that are adversely affected by aqueous solution. Colorants or flavoring agents can be added to the binding agent to prepare a granulation. Suitable lubricants for use in wet granulation include, but are not limited to, magnesium stearate, calcium stearate, stearic acid, wax, hydrogenated vegetable oil, talc, and starch.

In certain embodiments, matrices can be formed using a melt-granulation or melt-extrusion technique. Other ingredients can be added, for example, release modifying agents, diluents, lubricants, binders, granulating aids, colorants, flavorants, and glidants that are conventional in the pharmaceutical art in amounts up to e.g., about 50% by weight of the particulate if desired. The quantities of these additional materials should be sufficient to provide the desired effect to the desired formulation.

According to certain embodiments, methods of preparing dosage forms as disclosed herein can include a number of pharmaceutically acceptable preparation techniques (e.g., granulation, extrusion, compression, coating, encapsulation, etc. as disclosed above). In embodiments, methods of preparation include granulating one or more components to form a matrix. Compressing (and optionally shaping) the matrix to form one or more tablets using, for example, a tablet press. In certain embodiments, the blended powder formulation may then be encapsulated within a pharmaceutically acceptable capsule.

### Dosage Formats and Routes of Administration

### Dosage Formats

In certain embodiments, the mixed ingredients can be compressed into flat large tablets, caplets, enteric coated tablets; filled into soft capsules, hard capsules, capsule in a capsule, soft gel, or formed into beadlets, or pellets, referred to as precompression (or known as, slugging), or any other suitable form.

### Routes Of Administration

According to certain embodiments, dosage forms as disclosed herein are formulated for any suitable route of administration. In embodiments, the dosage forms comprise a route of administration selected from oral, parenteral, subcutaneous, intramuscular, intraperitoneal, intravenous, transdermal, transmucosal (e.g., via the transurethral or rectal routes), sublingual, buccal, gingival, rectal, subcutaneous, transpulmonary, topical, nasal, inhalation and combinations thereof.

### Methods of Use

Further disclosed herein are methods of using dosage forms and/or kits according to embodiments herein. In certain embodiments, disclosed are methods of supporting a human body's response and nutritional status to one or more of the following non-limiting mood disorders; any depressive type disorder including bipolar disorder, anti-psychotic type disorder, anhedonia, anxiety or stress type disorder, autism spectrum disorder, obsessive compulsive disorder, attention type disorder, or any mood disorder having nutrient deficits, that include administering to a human a dosage form, which may be a part of a kit, according to certain embodiments herein. In certain embodiments, the dosage form can be administered by the human as needed when the human is suffering from one of the aforementioned mood disorders. In certain embodiments, the dosage form can be administered by the human or caregiver in the morning afternoon or evening. According to certain embodiments, the human can be administered any safe and suitable amount. In certain embodiments, the human can be administered up to 1 serving, up to 2 servings, up to 3 servings, or up to 4 servings daily of the dosage form where each serving is, for example, 1 or 2 capsules, 1 or 2 tablets, 1 or 2 shots, etc.

### Restoring Nutritional Status to Homeostatic Levels and Allostasis Across Six Main Process Pathways

According to certain embodiments, disclosed are a composition of ingredients that may have additive and/or synergistic properties along with optional pharmaceutically acceptable excipients that provide a targeted effect within one or more of the six key process pathways, resulting in an all-encompassing coverage of the six key process pathways, that is delivered using a variety of dosage forms that have multi-modal ingredient properties to provide both short term symptomatic relief of the disorder, and long-term replacement of nutritional deficits with selected ingredients restoring the nutrient to homeostasis levels, rebalancing the six process pathways, providing replacement of nutrient deficits, improvement in mood, and significant benefits to the person. In certain embodiments, the formulation of the ingredients is comprised of both active and inactive ingredients which target up to the six process pathways, providing improvement in mood and restoring nutritional status to homeostatic levels, rebalancing one or more systems, improvement in mood and nutritional status, providing an enhanced overall response. It is understood that one compound can effect one or more of the process pathways and that one or more compounds can effect one process pathway.

### Administered Either As a Monotherapy or as an Adjunctive Therapy To Medications

According to certain embodiments disclosed is a composition of ingredients that may have additive and/or synergistic properties along with optional pharmaceutically acceptable excipients that provide a targeted effect within one or more of 6 key pathways, resulting in an all-encompassing coverage of one or more pathways, that is delivered orally by using a variety of dose delivery forms that have multi-modal ingredient properties to provide both short term symptomatic relief of the disorder, and long-term replacement of nutritional deficits with selected ingredients restoring the nutrient to homeostasis levels, rebalancing at least one system, providing improvement in mood, which is administered either as a monotherapy or adjunctive therapy to medications commonly used which are known to those skilled in the art treating a patient with a mood disorder, rebalancing up to 6 systems, improvement in mood and nutritional status providing an enhanced overall response.

### Administered As a Kit

According to certain embodiments, disclosed herein are kits comprising dosage forms in accordance with embodiments herein. The kit can include a container such as a but not limited to a multi-layer tablet, caplet, as a capsule in a capsule where various ingredients may be separated, or co-packaged as individual dose forms, where one part of the kit are a dosage formula of nutrients targeting one or more of the six process pathways, and the other, one or more pharmaceutical active ingredients that are standard of care in the targeted mood disorder for example, an SSRI for MDD or an anti-psychotic used for the treatment of schizophrenia. In accordance with various embodiments, the dosage form comprises at least one nutritional ingredient and at least one pharmaceutical ingredient, for example, an SSRI, as described herein. In certain embodiments, the dosage form comprises at least one or more nutritional ingredient components having effects within or more of the six process pathways, and at least one pharmaceutical ingredient component described herein. In further embodiments, the dosage form comprises at least two or more nutritional ingredient components when combined, and may have additive or synergistic effects within one or more of the six process pathways, and at least one pharmaceutical ingredient component described herein. In further embodiments, the dosage form comprises at least one or more nutritional ingredient components when combined, with at least one pharmaceutical ingredient component described herein, mitigates potential side effects of the pharmaceutical ingredient, for example, adjunct use of 1-methylfolate administered as a kit with an SSRI to correct folate levels affected by the SSRI, thus restoring folate homeostasis.

### Mood Disorders, Current Treatments and Unmet Nutritional Needs

### Depressive Disorders

The main types of depressive disorders include: Major Depressive Disorder (MDD), Persistent Depressive Disorder (PDD), Bipolar Depression, Seasonal Affective Disorder (SAD), and Atypical Depression; all with or without an anhedonia component to the depressive disorder, or anhedonia alone.

### Major Depressive Disorder (MDD)

Depression is one of the most prevalent mental disorders in the United States; more than 16% of individuals suffer from depression during their lifetime. Globally, it is the leading cause of disability and impacts more than 300 million people. Despite the prevalence and severity of the disease, its precise pathophysiology remains unclear. Because of considerable heterogeneity in the neurobiology and genetics of depression, there is a need for a variety of options in order to individualize treatment. Unfortunately, there are no clinically useful biomarkers to guide the selection of optimal treatment.

Usual Care and Adjunctive Therapies: Usual care for MDD includes treatments such as selective serotonin reuptake inhibitors (SSRIs) or serotonin and norepinephrine reuptake inhibitors (SNRIs) that target monoamine neurotransmitters, increasing their presence in the synapse by blocking their reuptake by transporters. Despite decades of use, monoaminergic therapies leave several unmet needs for patients: treatment onset may be slow, and many patients do not respond or remit to symptom levels that allow normal function; side effects can be significant; and many patients find it difficult to continue treatment and even discontinue antidepressant use, resulting in withdrawal reactions and/or appearance of new depressive symptoms.

The Sequenced Treatment Alternatives to Relieve Depression (STAR*D) study, funded by the National Institutes of Mental Health, found that approximately 30% of patients experienced remission from depression and less than 50% of patients achieved a 50% or greater reduction in baseline depressive symptomology scores with initial SSRI monotherapy. Furthermore, response and remission rates decreased after each failed treatment. Of the patients who received secondary treatments after failure of initial SSRI therapy, 18%, 21%, and 25% achieved remission with sertraline, sustained-release bupropion, and extended-release venlafaxine treatment, respectively. Only 10% to 16% achieved remission in their third phase of treatment. Thus, treatment of MDD with monotherapy may be incomplete for many patients, and there is a clear need for effective alternative or adjunctive therapies.

There are a number of commonly used adjunctive therapies available to patients for whom monoaminergic-based therapy is insufficient. Many of them are associated with potentially significant side effects that may put patients at higher risk for other serious health conditions.

While several are evidence-based, only the atypical antipsychotics Lumateperone (Caplyta®), Cariprazine (Vraylar®), Brexpiprazole (Rexulti®), Aripiprazole (Abilify®, Opipza®), and Quetiapine XR (Seroquel XR®), are currently approved by the FDA. In selecting adjunctive therapies, clinicians should consider the risk-benefit profile of each agent and individualize treatment accordingly. Available evidence suggests that two-thirds of patients taking drugs for depression require the use of at least one adjunctive therapy, with many patients cycling through therapy combinations before finding reasonably effective treatment, often a combination of polypharmacy and nonpharmacologic treatments. In particular, patients with treatment-resistant depression are known to experience worse outcomes than those who respond to usual care therapies. Longer duration of untreated depression and the presence of residual symptoms are associated with worse outcomes, including greater cognitive and functional impairment, as well as higher risk of relapse, morbidity, and even mortality. It is therefore important to identify the proper adjunctive therapy early in the treatment process to improve the possibility of acute full remission and increase the likelihood of longer-term efficacy. Advances in the understanding of depression etiology and pathophysiology may allow clinicians to better identify patients who will respond to particular therapies or adjunctive therapies Deficiencies in B vitamins (B6, B9, B12), omega-3 fatty acids, magnesium, vitamin D3 and zinc are commonly associated with MDD. These nutrients are crucial for neurotransmitter synthesis and regulation, inflammation control, and neuroprotection from oxidative stress and inflammation as well as the HPA stress response. Restoring these nutrient levels to homeostasis has shown potential in improving depressive symptoms and enhancing the efficacy of antidepressant treatments

### Other Depressive Disorders:

Persistent Depressive Disorder (PDD), also known as dysthymia, is a chronic form of depression with symptoms lasting for at least two years. It is less common than MDD but can be equally debilitating. Symptoms are similar to MDD but are generally less severe and more chronic. Treatment typically involves psychotherapy and antidepressants.

Bipolar Depression, characterized by episodes of depression and mania or hypomania. The depressive episodes are similar to those in MDD but are part of a broader mood disorder. Treatment includes mood stabilizers (e.g., lithium), antipsychotics, and sometimes antidepressants, though these must be used cautiously to avoid triggering manic episodes. Similar to MDD, PDD is also associated with deficiencies in B vitamins, vitamin D3, omega-3 fatty acids, and minerals like magnesium and zinc, among others. Addressing these deficiencies through diet or supplementation can help manage chronic depressive symptoms and improve overall treatment outcomes. Restoring these nutrient levels to homeostasis has shown potential in improving depressive symptoms and enhancing the efficacy of antidepressant treatment.

Seasonal Affective Disorder (SAD) is a type of depression that occurs at a specific time of year, usually in the winter when there is less natural sunlight. Symptoms include low energy, hypersomnia, overeating, weight gain, and a craving for carbohydrates. Light therapy, psychotherapy, and antidepressants are common treatments. Vitamin D deficiency is often implicated in SAD.

Atypical Depression is characterized by mood reactivity (mood brightens in response to positive events), significant weight gain or increased appetite, excessive sleep, a heavy feeling in the limbs, and a significant sensitivity to rejection. It is treated with psychotherapy and medications, including SSRIs and MAOIs.

Nutrient deficiencies commonly associated with depressive disorders include deficiencies in B vitamins (B6, B9, B12), omega-3 fatty acids, magnesium, zinc, and vitamin D.

### Nutrient Deficiencies in Depressive Disorders and Use of Four Specific Nutritional Ingredients to Improve Condition:

The administration of L-methylfolate, vitamin D3, zinc, and L-theanine can help improve depressive disorders through various mechanisms:

L-methylfolate: This is the bioactive form of folate that can cross the blood-brain barrier. It plays a crucial role in the synthesis of monoamines such as serotonin, norepinephrine, and dopamine, which are neurotransmitters involved in mood regulation. Supplementation with L-methylfolate enhances the efficacy of antidepressants, particularly in individuals with genetic polymorphisms affecting folate metabolism.

Vitamin D3: Vitamin D deficiency is associated with an increased risk of depression. Vitamin D3 has anti-inflammatory and neuroprotective properties, and it can modulate the synthesis of neurotransmitters and neurotrophic factors. Supplementation with vitamin D3 can improve depressive symptoms, potentially by preserving brain structural and functional connectivity.

Zinc: Zinc is an essential trace element that acts as an allosteric modulator of NMDA receptors and is involved in neuroplasticity and neurotransmission. Low serum zinc levels can be indicative of an individuals with depression. Zinc supplementation can enhance the antidepressant effects of standard treatments by modulating glutamatergic neurotransmission and reducing oxidative stress.

L-theanine: This amino acid, found in green tea, has anxiolytic and antidepressant properties. It can increase levels of GABA, serotonin, and dopamine in the brain, and can improve depressive symptoms when used as an adjunct to standard antidepressant therapy.

These supplements can enhance the efficacy of standard antidepressant treatments through various mechanisms, including neurotransmitter synthesis, anti-inflammatory effects, and modulation of neuroplasticity.

### Psychotic Disorders

The main types of psychotic disorders include Schizophrenia, Schizoaffective Disorder, Delusional Disorder, Brief Psychotic Disorder, and Schizophreniform Disorder.

### Schizophrenia

This is the most common psychotic disorder, with a lifetime prevalence of about 1%. Symptoms include hallucinations, delusions, disorganized speech, and negative symptoms such as blunted affect and avolition. Treatment typically involves antipsychotic medications, with second-generation antipsychotics being preferred due to fewer extrapyramidal side effects and symptoms.

### Schizoaffective Disorder

This disorder has features of both schizophrenia and mood disorders (depression or bipolar disorder). The prevalence is lower than schizophrenia, around 0.3%. Symptoms include a combination of psychotic symptoms (hallucinations, delusions) and mood symptoms (depression or mania). Treatment involves antipsychotics, mood stabilizers, and antidepressants.

### Delusional Disorder

This disorder is characterized by persistent delusions without other major psychotic symptoms. It is relatively rare, with a prevalence of about 0.2%. Symptoms include non-bizarre delusions that are plausible but untrue. Treatment typically involves antipsychotic medications and psychotherapy apply.

### Brief Psychotic Disorder:

This disorder involves sudden onset of psychotic symptoms that last less than one month, after which the individual returns to their previous level of functioning. It is rare, with a prevalence of about 0.1%. Symptoms include hallucinations, delusions, and disorganized speech or behavior. Treatment usually involves short-term use of antipsychotics and supportive therapy.

### Schizophreniform Disorder:

This disorder has symptoms similar to schizophrenia but lasts between one and six months. The prevalence is about 0.2%. Symptoms include hallucinations, delusions, disorganized speech, and negative symptoms. Treatment involves antipsychotic medications. As with schizophrenia, deficiencies in folate, vitamin D, and zinc can be addressed to improve outcomes.

### Nutrient Deficiencies in Psychotic Disorders and Use of Four Specific Nutritional Ingredients to Improve Condition:

Nutrient deficiencies in Psychotic Disorders, particularly in 1-methylfolate, vitamin D, and zinc, are common across psychotic disorders and can impact symptom severity and treatment outcomes. Addressing these deficiencies through supplementation can be a valuable adjunctive strategy in managing these conditions.

L-methylfolate: Folate deficiency, particularly in its active form 1-methylfolate, is associated with worse symptoms in psychotic disorders such as schizophrenia and schizoaffective disorder. A meta-analysis found significantly lower blood levels of folate in individuals with first-episode psychosis (FEP) compared to controls, and these lower levels were inversely related to psychiatric symptoms. Supplementation with 1-methylfolate can improve symptoms, particularly negative symptoms, in schizophrenia

Vitamin D: Vitamin D deficiency is prevalent among individuals with psychotic disorders, including schizophrenia, schizoaffective disorder, and others. Lower vitamin D levels are associated with more severe positive and negative symptoms. A systematic review and meta-analysis confirmed that patients with schizophrenia have significantly lower levels of vitamin D compared to controls, and this deficiency is linked to worse clinical outcomes. Supplementation with vitamin D can improve cognitive performance and reduce negative symptoms.

Zinc: deficiency can cause psychotic disorders, particularly schizophrenia. Individuals with schizophrenia can have significantly lower serum zinc concentrations as compared to healthy controls, with more pronounced reductions in newly diagnosed, drug-naïve patients. Zinc has a role in neurotransmitter function and immune response, and its deficiency may exacerbate psychotic symptoms.

L-theanine: This amino acid, has neuroprotective and anxiolytic properties. It can reduce positive symptoms, activation, and anxiety in patients with schizophrenia and schizoaffective disorder when used as an adjunct to antipsychotic treatment. L-theanine modulates neurotransmitter levels, including glutamate and GABA, which can help stabilize mood and reduce psychotic symptoms.

### Attention Disorders

Attention Deficit Hyperactivity Disorder (ADHD) is the primary type of attention deficit disorder, characterized by symptoms of inattention, hyperactivity, and impulsivity. ADHD is classified into three subtypes: predominantly inattentive presentation, predominantly hyperactive-impulsive presentation, and combined presentation.

Prevalence: ADHD affects approximately 7% of children and adolescents worldwide.

Symptoms: The symptoms of ADHD include difficulty sustaining attention, hyperactivity, impulsivity, restlessness, excessive talking, and difficulty organizing tasks.

Treatments: The first-line treatments for ADHD include stimulant medications such as methylphenidate and amphetamines, and non-stimulant medications like atomoxetine, guanfacine, and clonidine. Behavioral therapy and psychoeducation are also important components of treatment.

### Nutrient Deficiencies in Attention Disorders and Use of Four Exemplary Nutritional Ingredients to Improve Condition.

1. L-methylfolate: Folate is essential for brain function, and its deficiency could impact ADHD symptoms.
2. Vitamin D: Vitamin D supplementation can be associated with improvements in cognitive function and ADHD symptoms.
3 Zinc: Zinc deficiency can be addressed to improve ADHD symptoms.
4. L-theanine is a non-protein amino acid found in tea leaves, and has potential cognitive and neurophysiological benefits. Its administration may help improve conditions related to Attention Deficit Hyperactivity Disorder (ADHD) through several mechanisms:
5. Cognitive Function and Attention: L-theanine can enhance attention and cognitive performance. L-theanine can improve total cognition composite scores in children with ADHD when administered alone and in combination with caffeine. L-theanine administration can also improve neurophysiological measures of attention in a dose-dependent manner, particularly in tasks requiring selective attention.
6. Inhibitory Control: L-theanine in combination with caffeine can improve inhibitory control and sustained attention in children with ADHD.
7. Reduction of Mind Wandering: L-theanine can decrease mind wandering, which can enhance attention to target stimuli. L-theanine can reduce neural resource allocation to distractors, thereby improving focus on relevant tasks.
8. Anxiolytic Effects: L-theanine has anxiolytic properties, which can be beneficial for individuals with ADHD who often experience comorbid anxiety. It modulates neurotransmitters such as serotonin and dopamine, contributing to a calming effect that may help reduce hyperactivity and impulsivity.
9. Neuroprotective Effects: L-theanine increases levels of brain-derived neurotrophic factor (BDNF) and has neuroprotective properties, which may support overall brain health and cognitive function.

L-theanine can potentially improve ADHD-related symptoms by enhancing attention, reducing mind wandering, and providing anxiolytic and neuroprotective effects and its combination with caffeine may be particularly more effective in improving cognitive performance and inhibitory control in ADHD patients.

### Autism

Autism Spectrum Disorder (ASD) encompasses a range of neurodevelopmental conditions characterized by challenges with social skills, repetitive behaviors, speech, and nonverbal communication. The main types of ASD include Autistic Disorder, Asperger Syndrome, and Pervasive Developmental Disorder-Not Otherwise Specified (PDD-NOS). The prevalence of ASD is approximately 1 in 36 children, with a higher incidence in males.

Symptoms of ASD vary widely but generally include difficulties in social interaction, communication challenges, and a tendency to engage in repetitive behaviors.

Treatments for ASD are multifaceted and often include behavioral therapies, educational interventions, and sometimes medications to manage specific symptoms such as irritability or hyperactivity. There is no cure for ASD, but early intervention can significantly improve outcomes.

### Nutrient Deficiencies in Attention Disorders and Use of Four Specific Nutritional Ingredients to Improve Condition.

### Supplementation

Zinc: Supplementation can improve behavioral deficits in animal models of ASD and may benefit human patients.

Vitamin D: Supplementation can improve behavioral functioning in ASD, particularly when combined with omega-3 fatty acids.

L-methylfolate: Folate is essential for brain development; the specific benefits of L-methylfolate supplementation may be beneficial.

L-theanine: This compound may have potential benefits through its effects on the HPA stress response and glutamate, both of which could exacerbate the condition.

In certain embodiments, addressing nutrient deficiencies through targeted supplementation of L-methylfolate, zinc, vitamin D, and L-theanine may offer therapeutic benefits for individuals with ASD.

### Stress and Anxiety Disorders

The main types of stress and anxiety disorders include Generalized Anxiety Disorder (GAD), Social Anxiety Disorder (SAD), and Panic Disorder (PD). These disorders have a lifetime prevalence of approximately 34% in the US, with GAD at 6.2%, SAD at 13%, and PD at 5.2%.

### Symptoms

Generalized Anxiety Disorder (GAD): Excessive worry, restlessness, fatigue, difficulty concentrating, irritability, muscle tension, and sleep disturbances.

Seasonal Affective Disorder (SAD): Intense fear of social situations, worry about being judged, avoidance of social interactions, and physical symptoms like sweating and trembling.

Panic Disorder (PD): Recurrent unexpected panic attacks, palpitations, shortness of breath, dizziness, and fear of losing control.

First-line treatments include pharmacotherapy with SSRIs (e.g., sertraline) and SNRIs (e.g., venlafaxine extended release), and Cognitive Behavioral Therapy (CBT). CBT is particularly effective, showing large effect sizes in treating GAD and moderate effects in SAD and PD. Pharmacotherapy with SSRIs and SNRIs has shown small to medium effect sizes compared to placebo.

Clinical guidelines generally recommend SSRIs and SNRIs as first-line treatments for anxiety disorders. Benzodiazepines (BZs) are a class of medications commonly used for the treatment of anxiety disorders. BZs are often reserved for short-term use or as adjunctive therapy when rapid symptom control is needed while waiting for the effects of SSRIs or SNRIs to take hold. For example, the American College of Obstetricians and Gynecologists (ACOG) advises that BZs should be used sparingly and preferably only as a bridge until other treatments become effective.

### Nutrient Deficiencies in Attention Disorders and Use of Four Exemplary Nutritional Ingredients to Improve Condition

1. L-methylfolate: Supplementation with L-methylfolate can improve symptoms of depression and anxiety, particularly when used as an adjunct to standard treatments.
2. Vitamin D: Vitamin D supplementation may alleviate symptoms of anxiety, especially in individuals with vitamin D deficiency.
3. Zinc: Zinc supplementation can have potential benefits in reducing anxiety, likely due to its role in neurotransmitter function and neurogenesis.
4. L-theanine may exert its anxiolytic effects by modulating neurotransmitter levels in the brain, particularly increasing levels of GABA, serotonin, and dopamine, which are associated with relaxation and mood regulation. Additionally, it may reduce levels of excitatory neurotransmitters like glutamate, contributing to its calming effects. L-theanine may reduce anxiety symptoms. For example, daily doses of 200-400 mg of L-theanine can help reduce stress and anxiety in individuals exposed to stressful conditions. Also, L-theanine supplementation may reduce anxiety symptoms in patients with schizophrenia and schizoaffective disorder when used as an adjunct to antipsychotic treatment.

In summary, anxiety disorders are prevalent and significantly impact quality of life. Effective treatments include SSRIs, SNRIs, Benzodiazepines, and CBT. Nutrient deficiencies, particularly in L-methylfolate, vitamin D, and zinc, may exacerbate symptoms, and supplementation can be a beneficial adjunct to standard treatments.

### OTHER EMBODIMENTS

According to certain embodiments, a composition of ingredients disclosed herein may have additive and/or synergistic properties along with optional pharmaceutically acceptable excipients that provide a targeted effect within one or more of six key process pathways, resulting in coverage of up to the six key process pathways, that is delivered orally using a dose delivery forms that have multi-modal ingredient properties to provide both short term symptomatic relief of the disorder, and long-term replacement of nutritional deficits with selected ingredients restoring the nutrient to homeostasis levels, rebalancing the six systems, providing significant benefits to the patient and improvement in mood for example, a patient with one or more a disorder including but not limited to; a depressive disorder, a psychotic disorder, anxiety disorder, attention disorder, or obsessive compulsive disorder, rebalancing the one or more systems, improvement in mood and nutritional status providing an enhanced overall response.

### Six Process Pathways Implicated in Mood Disorders:

A challenge in dealing with this disease is the frequent recurrence, treatment ineffectiveness, and the absence of diagnosis and treatment options, particularly in low- and middle-income countries. Nevertheless, there are viable treatment approaches, notably psychotherapy and/or the use of antidepressants. Both demonstrate efficacy in addressing MDD. Still, approximately 30% of individuals affected by MDD do not experience remission, even after multiple treatments.

Despite the complex nature of MDD, several studies support the importance of various factors, including:
1. Neurotransmitter synthesis and release involved in neural communication
2. Glutamate modulation
3. Oxidative stress modulation
4. Inflammation modulation
5. Neurotrophic and Neuronal plasticity
6. Stress response modulation

### Neurotransmitter (Monoamine) Synthesis and Release Involved in Neural Communication Normal Neurotransmitter Processes

The synthesis and release of monoamines, such as dopamine, norepinephrine, and serotonin, involve several key mechanisms and mediators.

Synthesis: Monoamines are synthesized from amino acid precursors. For instance, dopamine is synthesized from tyrosine, which is converted to L-DOPA by tyrosine hydroxylase and then to dopamine by aromatic L-amino acid decarboxylase. Similarly, serotonin is synthesized from tryptophan via tryptophan hydroxylase and aromatic L-amino acid decarboxylase.

Storage and Release: Monoamines are stored in synaptic vesicles by vesicular monoamine transporters (VMATs). VMATs use a proton gradient to transport monoamines into vesicles, concentrating them for release upon neuronal firing. Upon an action potential, these vesicles fuse with the presynaptic membrane, releasing their contents into the synaptic cleft.

Reuptake and Degradation: After release, monoamines are cleared from the synaptic cleft primarily by reuptake transporters such as the dopamine transporter (DAT), norepinephrine transporter (NET), and serotonin transporter (SERT). These transporters reabsorb monoamines back into the presynaptic neuron, where they can be repackaged into vesicles or degraded by enzymes like monoamine oxidase (MAO).

Receptors: Monoamines exert their effects by binding to specific receptors on target cells. These receptors include G protein-coupled receptors (GPCRs) and ionotropic receptors. For example, dopamine acts on D1-like and D2-like receptors, which are GPCRs that modulate various intracellular signaling pathways. Serotonin receptors, such as 5-HT1A and 5-HT2A, also belong to the GPCR family and regulate neurotransmitter release and neuronal excitability.

Regulation: The release of monoamines is modulated by presynaptic autoreceptors, which provide feedback inhibition. For instance, dopamine D2 autoreceptors inhibit further dopamine release, while serotonin 5-HT1B autoreceptors inhibit serotonin release.

These mechanisms collectively ensure the precise regulation of monoamine levels, which is crucial for maintaining normal neurological function and behavior.

### Dysregulation of the Neurotransmitter (Monoamine) Process Pathway

The pathophysiologic mechanisms and key mediators involved in the synthesis and release of monoamines, such as serotonin (5-HT), norepinephrine (NE), and dopamine (DA), play a significant role in the development of mood disorders like major depressive disorder (MDD).

Synthesis and Release: Monoamines are synthesized from amino acid precursors (e.g., tryptophan for serotonin, tyrosine for dopamine and norepinephrine) and stored in synaptic vesicles. Upon neuronal activation, these vesicles release monoamines into the synaptic cleft, where they bind to their respective receptors on postsynaptic neurons.

Dysregulation in Mood Disorders: In MDD, there is often a dysfunction in the monoaminergic systems. This can involve reduced presynaptic release of these neurotransmitters or impaired signal transduction, leading to altered receptor function and intracellular signaling. For example, decreased levels of serotonin and norepinephrine have been observed in the brains of individuals with depression, which may be due to increased activity of monoamine oxidase A (MAOA), an enzyme that degrades these neurotransmitters.

Receptor and Signal Transduction Abnormalities: Abnormalities in receptor function, such as altered binding and regulation of 5-HT, NE, and DA receptors, have been implicated in mood disorders. For instance, disruptions in 5-HT1A receptor complexes can lead to reduced neuroplasticity and contribute to depressive symptoms. Additionally, the interaction between different monoamine systems, such as the inhibitory effects of serotonin on dopamine and norepinephrine systems via 5-HT2A and 5-HT2C receptors, further complicates the pathophysiology.

Neuroplasticity and Neurogenesis: Impaired neurogenesis and neuroplasticity, particularly in the hippocampus, are also critical factors in the pathophysiology of depression. This impairment is often linked to reduced levels of brain-derived neurotrophic factor (BDNF) and other neurotrophic factors, which are influenced by monoaminergic activity.

Therapeutic Implications: Antidepressants that target monoaminergic systems, such as selective serotonin reuptake inhibitors (SSRIs) and serotonin-norepinephrine reuptake inhibitors (SNRIs), aim to restore the balance of these neurotransmitters and improve mood symptoms by enhancing monoamine availability and receptor function.

In summary, the pathophysiology of mood disorders involves complex interactions between monoamine synthesis, release, receptor function, and intracellular signaling, all of which contribute to the regulation of mood and emotional behavior.

Nutritional Modulators that Target Neurotransmitter (Monoamine) Modulators:

Folate and L-Methylfolate: Folate is crucial for the synthesis of neurotransmitters. L-methylfolate, the bioactive form of folate, can cross the blood-brain barrier and has been shown to enhance the efficacy of antidepressants in individuals with low folate levels. Folate deficiencies may cause depressive symptoms by elevating homocysteine and intracellular one-carbon metabolism.

Folate is crucial for the synthesis of neurotransmitters. L-methylfolate, the bioactive form of folate, can cross the blood-brain barrier and has been shown to enhance the efficacy of antidepressants in individuals with low folate levels.

Vitamin D: Deficiency in vitamin D is associated with depression. Supplementation has been shown to improve depressive symptoms, potentially through its role in neuroimmune modulation and neuroplasticity.

Magnesium: Both minerals play roles in neurotransmitter function and synaptic plasticity. Supplementation with zinc and magnesium has been shown to reduce depressive symptoms, particularly in individuals with deficiencies.

The one-carbon cycle is critical to numerous transmethylation processes in the CNS and to the metabolism of monoamine neurotransmitters such as serotonin, norepinephrine, and dopamine. Folic acid must first be converted by 5,10-methylenetetrahydrofolate reductase (MTHFR) to the metabolically active form, L-5-methyl-tetrahydrofolate (MTHF), which is the only form of folate that crosses the blood-brain barrier (BBB). Upon entering the CNS, MTHF acts as the methyl donor in numerous methylation-dependent processes, including the methylation of homocysteine to form methionine and S-adenosylmethionine (SAMe). Methionine is an essential amino acid and acts as the substrate for SAMe, a methyl group donor in more than 100 methylation reactions in the body. Methionine is also a precursor to glutathione, a naturally occurring antioxidant that modulates glutamate activity and maintains cellular oxidative balance by scavenging and neutralizing reactive oxygen and nitrogen species. Augmenting antidepressants with folate may benefit MDD patients who are predisposed to or already have low folate levels. Folic acid supplementation can improve depressive symptoms, cognition, and oxidative imbalances, and induce hippocampal neurochemical changes. Despite these positive outcomes, L-methylfolate (i.e., L-5-methyl-tetrahydrofolate) may be a better alternative than folic acid due to its ability to pass the BBB. Unlike folic acid, L-methylfolate does not carry the risk of masking symptoms of vitamin B12 deficiency and may have fewer drug interactions that inhibit dihydrofolate reductase.

S-adenosylmethionine (SAMe) can be considered an initial MDD treatment for patients who prefer complementary or alternative approaches; however, it can induce mania in depressed patients on the bipolar spectrum. SAMe is a naturally occurring methyl donor involved in over 100 methyltransferase reactions for critical metabolic pathways, including methylation of DNA bases, proteins, phospholipids, free amino acids, catecholamines, and neurotransmitters. DNA methylation acts to turn off gene transcription, while demethylation is linked to transcriptional activation; therefore, aberrant methylation can negatively impact CNS disorders. SAMe is generated from L-methionine in the one-carbon cycle, which is dependent upon sufficient levels of folate and vitamin B12, both of which are linked to depressive symptoms. There are several mechanisms potentially responsible for the antidepressant activity of SAMe. As a donor of methyl groups, SAMe may exert antidepressant effects by methylation of plasma phospholipids, altering neuronal membrane fluidity and the function of membrane proteins, including monoamine receptors and transporters. Lower SAMe levels may cause a decrease in monoamine synthesis, thereby increasing the risk of depression. In animal models, increased SAMe levels were positively correlated with monoamine neurotransmitter concentrations in the brain. SAMe may be taken orally up to 1600 mg/d, a significantly bioavailable and non-toxic dose. Patients receiving SAMe 800 mg/day in addition to their normal antidepressant have shown significantly greater changes in HAM-D scores from baseline and higher remission rates compared with placebo (p<0.05) (Table 1). In a systematic review, all studies demonstrated a significantly positive effect for SAMe up to 1600 mg on the HAM-D in MDD. SAMe produces a rapid onset of action, with nearly a 5-point difference in HAM-D scores from baseline to week 1. In a Cochrane systematic review, eight trials that included 934 participants investigated the effects of SAMe versus placebo or SSRIs in MDD. Compared with placebo, SAMe was more efficacious as an adjunctive treatment in terms of response and remission; however, the level of evidence was low. SAMe is generally well tolerated and has a favorable safety profile. Frequently reported adverse events associated with SAMe are nausea, diarrhea, and abdominal discomfort. As previously mentioned, SAMe may induce mania and hypomania, even in patients without a previous history of bipolar disorder.

Vitamin B12 is critical to central nervous system (CNS) development and functions by acting as cofactors in converting homocysteine to methionine, an essential amino acid involved in numerous methylation processes critical for synthesizing proteins, lipids, nucleic acids, neurotransmitters, and hormones. Vitamin B12 deficiency is known to disrupt infant brain development and cause neural tube defects, supporting its connection to brain function. Altered vitamin B12 levels are also associated with issues well beyond infancy, particularly with inflammatory conditions that result in psychiatric disorders, including depressive disorders. Inflammation and depressive symptoms have a bidirectional relationship, facilitating and promoting one another, as evidenced by elevation in various inflammatory biomarkers in a subgroup of susceptible MDD patients. From a mechanistic standpoint, inflammation can trigger microglial activation and subsequent release of proinflammatory cytokines, which can induce depressive symptoms by altering the production, metabolism, and transport of neurotransmitters that affect mood (e.g., dopamine, glutamate, serotonin).

Magnesium reduces the expression of pro-inflammatory cytokines such as interleukin-1ß (IL-1β), tumor necrosis factor-alpha (TNF-α), and interleukin-6 (IL-6). This suppression is mediated through the inhibition of signaling pathways like NF-κB and the activation of anti-inflammatory pathways such as PPARY; helping to decrease the production of reactive oxygen species (ROS), which are harmful byproducts that contribute to oxidative stress and inflammation. By reducing ROS levels, magnesium protects neuronal cells from damage and inhibits the activation of microglia, the brain's resident immune cells that play a crucial role in neuroinflammation. This inhibition helps to prevent the release of inflammatory mediators and supports a healthier neural environment; helps to preserve the integrity of the blood-brain barrier, which is essential for preventing the infiltration of inflammatory cells and molecules into the brain. This protective effect reduces neuroinflammation and supports overall brain health; and activates signaling pathways such as ERK1/2 and PPARγ, which are involved in neuroprotection and anti-inflammatory responses. These pathways help to reduce inflammation and promote neuronal survival.

Zinc: has many important effects contributing to maintaining optimal neurotransmitter function and synaptic plasticity. Supplementation with zinc and magnesium has been shown to reduce depressive symptoms, particularly in individuals with deficiencies. Zinc modulates monoamine neurotransmitters through various mechanisms, including the following non-limiting examples: inhibition of type A monoamine oxidase (MAO-A) activity, which is responsible for the breakdown of serotonin and norepinephrine. This inhibition increases the availability of these neurotransmitters in the brain, contributing to improved mood and reduced depressive symptoms; enhances the uptake of serotonin (5-HT) in certain brain regions, such as the corpus callosum, cingulate cortex, and Raphe nucleus, is concentration-dependent and can counteract the inhibitory effects of some antidepressants on serotonin uptake, thereby influencing extracellular serotonin levels; increases the density of 5-HT1A and 5-HT2A receptors in the hippocampus and frontal cortex, respectively, where upregulation enhances serotonergic signaling, which is associated with antidepressant effects; affects dopamine metabolism by increasing tissue levels of dopamine metabolites such as DOPAC and HVA in the prefrontal cortex, modulation of dopaminergic neurotransmission can influence mood and cognitive functions. These mechanisms highlight zinc's role in modulating monoamine neurotransmitter levels, a crucial mechanism underlying its antidepressant and neuroprotective effects. Zinc modulates the hypothalamic-pituitary-adrenal (HPA) stress response through several mechanisms, including the following non-limiting examples: zinc deficiency has been shown to enhance HPA axis activity, leading to increased glucocorticoid secretion, such as corticosterone. This heightened glucocorticoid secretion is linked to behavioral abnormalities and depressive symptoms; zinc is necessary for proper glucocorticoid receptor function. Adequate zinc levels help maintain the sensitivity of these receptors, which are crucial for the negative feedback regulation of the HPA axis. This feedback mechanism helps to prevent excessive glucocorticoid release during stress; modulates neurotransmitter systems, including glutamate and GABA, which are involved in the stress response; affects the levels of BDNF, a neurotrophin involved in neuronal survival and plasticity, wherein, adequate zinc levels support BDNF signaling, which can mitigate the adverse effects of stress on the brain; reduction of inflammatory markers due to its anti-inflammatory properties that help reduce the levels of pro-inflammatory cytokines such as IL-6 and TNF-α, which are elevated during stress, wherein a reduction in inflammation can help modulate the HPA axis response to stress;

Magnesium and zinc: play roles in neurotransmitter function and synaptic plasticity. Supplementation with zinc and magnesium appears to have additive effects when administered together, which has been shown to reduce depressive symptoms, particularly in individuals with deficiencies.

Vitamin D: Deficiency in vitamin D is associated with depression. Supplementation has been shown to improve depressive symptoms, potentially through its role in neuroimmune modulation and neuroplasticity.

Vitamin D and Zinc: combined effects of Vitamin D3 and Zinc has shown that, when these two ingredients are administered together, they have demonstrated significant improvement in people with depression, highlighting the additive effects of these two nutrients.

Omega-3 Fatty Acids are primarily eicosapentaenoic acid (EPA) and docosahexaenoic acid (DHA). They modulate neuroinflammation, neurotransmitter function, and neuroplasticity. Omega-3 supplementation can reduce depressive symptoms, particularly when used as an adjunct to antidepressant therapy.

Probiotics: Gut dysbiosis is linked to depression through the gut-brain axis. Probiotic supplementation can improve mood by reducing systemic inflammation and modulating neurotransmitter production.

Four Exemplary Nutritional Ingredients and MOA(s) within the Process Pathway for the Composition:
(a) L-methylfolate: This bioactive form of folate is crucial for the synthesis of monoamine neurotransmitters such as serotonin, dopamine, and norepinephrine. It acts as a cofactor in the conversion of homocysteine to methionine, which is a precursor for S-adenosylmethionine (SAMe), a key methyl donor in the synthesis of these neurotransmitters. By enhancing monoamine synthesis, L-methylfolate can potentially improve mood and reduce anxiety symptoms.
(b) Vitamin D3: This compound can influence the brain's dopamine system. It can enhance dopamine synthesis and release, and modulate dopamine transporter activity, which is crucial for maintaining dopamine levels in the synaptic cleft. Additionally, vitamin D3 supplementation can increase serotonin levels, helping alleviate anxiety symptoms.
(c) Zinc: Zinc can modulate neurotransmitter systems, including the serotonergic, dopaminergic, and glutamatergic systems. It can enhance the function of GABA, an inhibitory neurotransmitter, and modulate NMDA receptors, which mediate excitatory neurotransmission. Zinc deficiency can increase anxiety, and supplementation can help restore normal neurotransmitter function and reduce anxiety symptoms.
(d) L-theanine: This amino acid can modulate levels of serotonin, dopamine, and norepinephrine in various brain regions, including the prefrontal cortex, nucleus accumbens, and hippocampus. L-theanine has been shown to increase GABA levels and reduce glutamate levels, promoting relaxation and reducing anxiety. Its anxiolytic effects are likely mediated through these changes in neurotransmitter levels and activity.

In summary, L-methylfolate, vitamin D3, zinc, and L-theanine can modulate monoamine neurotransmitters, which may help manage anxiety by improving the balance of excitatory and inhibitory signals in the brain.

### Glutamate Modulation

### Normal Glutamate Processes

Glutamatergic signaling plays a vital role in the release of neurotrophic factors and neuroplasticity. Neuroplasticity allows for learning and memory and is essential for neuronal systems to adapt to environmental stimuli and tailor appropriate responses.

Glutamate signaling plays a complex role in synaptogenesis and neural plasticity. While glutamate was known to be present in the central nervous system, it was not acknowledged as a true neurotransmitter until 1984. Since then, glutamate has been established as the major excitatory neurotransmitter of the nervous system, mediating fast excitatory transmission in the brain. Glutamate is both, the most common amino acid in the brain, and the most abundant neurochemical agonist in the central nervous system. Glutamate signaling involves not only traditional presynaptic to postsynaptic transmission but also recycling and regulation of glutamate by glial cells via excitatory amino acid transporters. Vesicular glutamate transporters package presynaptic glutamate into vesicles within the neuron before release into the synaptic cleft.

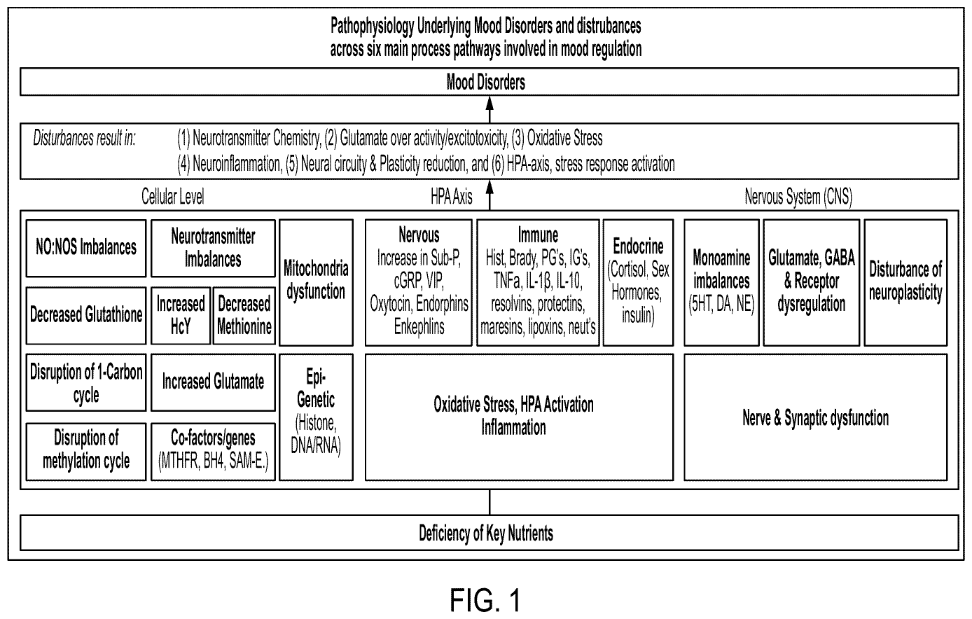

Receptors in the glutamate signaling cascade include ionotropic receptors (which act quickly, opening upon agonist binding and enable ion flow) and metabotropic receptors (which are G-protein-coupled receptors that help modulate synaptic activity and plasticity over a longer timescale) (FIG. 1) These receptor types are complex with a broad range of eff and downstream targets.

The ionotropic multi-subunit NMDA receptor has the highest affinity for glutamate. The NMDA receptor is tightly regulated and requires glutamate and glycine co-agonists for activation. Upon activation, the NMDA receptor ion channel is no longer blocked by the voltage-dependent pore blocker Mg2+, allowing for the influx of Ca2+ and Na+ cations into the postsynaptic neuron. This influx of calcium can trigger multiple signaling cascades that promote cell survival and growth, including the phosphoinositide 3 kinase-protein kinase B (PI3K-AkT) pathway, mammalian target of rapamycin (mTOR) pathway, and brain-derived neurotrophic factor (BDNF) release.

The mTOR pathway regulates protein translation and synaptic plasticity. mTOR is inhibited by tuberous sclerosis complexes and may be activated when PI3K-AkT or extracellular signal-regulated kinases (ERK) inhibit these complexes. mTOR interacts downstream with ribosomal protein S6 kinases, which regulate protein biosynthesis by inhibiting the phosphorylation of eukaryotic elongation factor 2 (eEF2), thereby allowing protein translation. mTOR activation thus increases the expression of synaptic proteins vital for the formation, maturation, and function of synaptic spines, thereby increasing dendritic spine density. Additionally, mTOR processes impact behavioral plasticity, cognition, and neuronal excitability and survival. BDNF is critical in the survival of neurons in adult brains and in synaptic plasticity. Binding of BDNF to tropomyosin receptor kinase B (TrkB) receptors enhances neuronal survival, synaptogenesis, and plasticity and further activates kinase pathways to increase mTOR signaling.

The α-amino-3-hydroxy-5-methyl-4-isoxazolepropionic acid (AMPA) receptor (AMPAR) is also a multi-unit ionotropic receptor that is widely expressed in the central nervous system. Glutamate has a lower affinity for ionotropic AMPARs than for NMDA receptors, which results in quicker inactivation of AMPARs than of NMDA receptors. Activation of the AMPAR results in changes in AMPAR trafficking and localization within the cell, which can lead to long-term potentiation. Additionally, activation of AMPAR by glutamate activates calcium channels and triggers the mTOR pathway.

Metabotropic glutamate receptors (mGluRs) trigger downstream effects by recruiting and activating G-proteins. Glial cells can express mGluRs, in addition to metabolizing glutamate to glutamine and releasing the NMDA receptor co-agonist d-serine. mGluRs are often located outside synapses on neurons and glia, yet they can still modulate synaptic activity and plasticity. Both ionotropic and metabotropic receptors interact with postsynaptic scaffold proteins, including postsynaptic density protein of 95 kDA (PSD-95), which mechanically stabilizes synapses. γ-Aminobutyric acid (GABA) is the major inhibitory neurotransmitter and is essential to balance excitatory glutamate neurotransmission. Excessive glycine, which can be released by both glial cells and glutamatergic neurons, can induce NMDA receptor endocytosis and depress NMDA receptor response.

### Glutamate Dysregulation

A preclinical study in 1990 first indicated that NMDA receptor antagonists mimicked the clinical effects of antidepressants in murine models, leading to the glutamate hypothesis of depression. Subsequent animal models have further supported that glutamatergic dysfunction plays a role in the mechanism of depression.

Chronic stress models in rodents are used to understand how the brain translates stress into depressive-like symptoms and physical changes in brain structure. Under chronic stress, neurons have impaired synaptic number and function as well as diminished plasticity, which increases susceptibility to depression. The synaptic alterations from chronic stress may include loss of mature spines and synaptic connections. The altered synaptic structure has been observed in people with depression. In a rodent model, upon treatment with the NMDA receptor antagonist ketamine, synaptic protein expression was rapidly restored, reversing structural and functional deficits within 24 hours. Stress and depression can also dysregulate GABA function, which causes imbalances between excitatory and inhibitory activity.

BDNF signaling, downstream of glutamate activation of AMPAR, is decreased in the brains of people with depression. Traditional antidepressants slowly induce BDNF expression, which enhances synaptic plasticity and subsequent symptom response. Rodent models support the importance of AMPAR activation and increased BDNF as essential for depression improvement with ketamine, as antidepressant effects are not observed if BDNF is neutralized or AMPAR inhibited. BDNF also plays a role in anxiety, schizophrenia, and other neurodegenerative disorders.

Human imaging and tissue studies confirm there are changes in the glutamatergic system in people with depression. Positron emission tomography (PET) imaging studies and postmortem tissue analyses have revealed abnormalities in NMDA receptor and mGluR subunit/protein expression in the prefrontal cortex in patients with MDD. Additionally, a meta-analysis of proton magnetic resonance spectroscopy neuroimaging studies found decreased glutamatergic metabolite concentrations in the medial frontal cortex of people with depression compared with healthy people. Glutamatergic dysregulation in MDD is also reflected by a significant (P=8.5×10−5) elevation in blood glutamate levels in people with MDD.

The fundamental role of glutamatergic signaling in depression in humans was clinically supported in 2000 in the first small (N=7), placebo-controlled, double-blind clinical trial of ketamine that demonstrated significance (P≤0.001 at 72 h), rapid improvement in major depression symptoms compared with placebo. The mechanism underlying this rapid action has been heavily investigated in preclinical models. As a high-affinity NMDA receptor antagonist, ketamine is theorized to trigger a surge of presynaptic glutamate release, which then activates AMPARs. This activation initiates an intracellular signaling cascade that increases BDNF and mTOR activity, promoting neuronal recovery. Clinically, a wide range of imaging studies, predominantly functional magnetic resonance imaging, have confirmed that ketamine treatment is associated with alterations within and between intrinsic connectivity networks implicated in depression.

Nutritional Modulators that Target Glutamate Process Pathways

To regulate glutamate-to-gamma-aminobutyric acid (GABA) modulation and potentially improve symptoms of mood disorders, certain nutritional ingredients can be beneficial.

Gamma-aminobutyric acid (GABA) itself is a key inhibitory neurotransmitter that can be consumed through dietary sources or enriched foods. Foods naturally containing GABA include fermented products like kimchi, miso, and certain types of yogurt, as well as green tea and some vegetables, or as a supplement.

Glutamine, a precursor to glutamate, is another important nutrient. It plays a crucial role in the glutamate-GABA cycle, and supplementation with glutamine has been shown to ameliorate depressive behaviors by increasing glutamatergic neurotransmission in the medial prefrontal cortex.

Probiotics such as Streptococcus thermophilus and Lactobacillus rhamnosus have been shown to modulate gut microbiota and increase serum levels of neurotransmitters, including GABA, which can positively affect mood and stress levels.

L-methylfolate, the bioactive form of folate, modulates glutamate and glutathione levels through several mechanisms, including the following non-limiting examples: plays a crucial role in the synthesis of neurotransmitters, including glutamate; acts as a cofactor in the conversion of homocysteine to methionine, which is subsequently converted to S-adenosylmethionine (SAMe). SAMe is essential for the methylation processes that regulate the synthesis and metabolism of neurotransmitters, including glutamate. By supporting these metabolic pathways, L-methylfolate helps maintain balanced glutamate levels, reducing the risk of excitotoxicity, and contributes to the synthesis of glutathione, a major antioxidant in the brain. It does this by providing the methyl groups necessary for the production of SAMe, which, in turn, supports the synthesis of cysteine, a precursor of glutathione. Increased availability of cysteine enhances the synthesis of glutathione, thereby boosting the brain's antioxidant defenses and reducing oxidative stress; inhibits the activity of enzymes such as GSK-3β and iNOS, which are involved in inflammatory pathways indirectly supporting the maintenance of glutamate and glutathione levels, as chronic inflammation can disrupt these pathways and lead to neurotoxicity.

Other B vitamins particularly B6, and B12, are essential for neurotransmitter synthesis and function. B vitamins are involved in the conversion of glutamate to GABA and have been associated with improved mood and reduced symptoms of depression.

Vitamin D modulates glutamate and GABA levels through several mechanisms: upregulation of the expression of glutamate transporters such as EAAT3 (SLC1A1), which enhances the uptake and clearance of glutamate from the synaptic cleft, helping to prevent excitotoxicity; decreases the exocytotic release of glutamate helping to balance excitatory neurotransmission and protect against excitotoxicity; influences the expression of GABA transporters such as GAT-3, which affects the uptake and release of GABA helping to maintain appropriate levels of GABA; and decreases in the exocytotic release of both glutamate and GABA.

Zinc modulates glutamate and GABA levels in the brain through several mechanisms, which are closely linked to its anti-inflammatory effects, including but not limited to: Reduction of glutamate release by activating presynaptic ATP-sensitive potassium (KATP) channels. This action helps to restore membrane potential and reduce excessive glutamate release, which can be neuroprotective by preventing excitotoxicity. Additionally, zinc can inhibit NMDA receptors, which are involved in glutamate signaling, thereby reducing excitatory neurotransmission; zinc increases the release of GABA, the primary inhibitory neurotransmitter, by potentiating AMPA/kainate receptors in the hippocampus, leading to an increase in GABAergic activity, which helps to balance excitatory (glutamate) and inhibitory (GABA) neurotransmission. Zinc also inhibits GABA transporters, such as GAT4, which reduces the reuptake of GABA and increases its availability in the synaptic cleft; zinc can inhibit GABAA receptors, particularly those that are extrasynaptic and neurosteroid-sensitive. This inhibition can modulate tonic inhibition and affect overall neuronal excitability. These actions of Zinc on glutamate and GABA levels contribute to its anti-inflammatory effects by reducing excitotoxicity and promoting inhibitory neurotransmission, thereby maintaining a healthier neural environment and supporting neuroprotection.

L-theanine modulates glutamate and GABA levels through several mechanisms; inhibits the transport of glutamine into neurons and astroglia, which in turn reduces the conversion of glutamine to glutamate by glutaminase decreasing the overall pool of glutamate available for neurotransmission, thereby reducing excitatory signaling; inhibits the release of glutamate in the brain partly due to its antagonistic action on NMDA receptors, which are involved in excitatory neurotransmission, inhibiting these receptors helps to prevent excitotoxicity; increases the release of GABA, increasing GABAergic activity balance excitatory and inhibitory neurotransmission, promoting a calming effect and reducing anxiety; and increase the expression of GABA receptors enhancing the inhibitory effects of GABA.

Four Exemplary Nutritional Ingredients and MOA(s) within Process Pathway for the Composition:
1. L-methylfolate modulates glutamate & NMDA receptors and restores optimal glutathione levels, lowering glutamate levels.
2. Vitamin D3 modulates the enzyme that converts glutamate to GABA, reducing glutamate levels and preventing excitotoxicity.
3. Zinc has been shown to have a key role in regulating glutamate and GABA receptor activation and inactivation.
a. L-theanine modulates glutamate and GABA levels through several mechanisms.

### Oxidative Stress Modulation

### Normal Oxidative Stress Processes

The body modulates oxidative stress within the nervous system through a combination of cellular and molecular mechanisms involving both neurons and glial cells. Neurons and astrocytes play distinct but complementary roles in maintaining redox homeostasis.

Astrocytes are crucial for providing antioxidant support to neurons. They achieve this through the activation of the Nrf2 (nuclear factor erythroid 2-related factor 2) pathway, which regulates a wide array of antioxidant genes. This pathway is robust in astrocytes but weak in neurons, making astrocytes essential for the overall antioxidant defense in the brain. The Nrf2 pathway in astrocytes leads to the upregulation of enzymes such as NAD (P) H: quinone oxidoreductase-1 and heme oxygenase-1, which have significant anti-inflammatory and antioxidant effects.

Neurons, on the other hand, rely on synaptic activity to regulate their antioxidant defenses. Synaptic activity can increase the production of reactive oxygen species (ROS), but it also enhances the antioxidant capacity of neurons through systems such as glutathione and the thioredoxin-peroxiredoxin pathway. This activity-dependent regulation allows neurons to adapt to varying levels of oxidative stress.

Additionally, the brain's redox balance is maintained by various antioxidant molecules and enzymes, including glutathione, superoxide dismutase, and catalase. These antioxidants neutralize ROS and prevent oxidative damage to cellular components.

In summary, modulation of oxidative stress in the nervous system involves a coordinated effort between neurons and astrocytes, with astrocytes playing a key role through the Nrf2 pathway and neurons adapting their antioxidant defenses to synaptic activity. This intricate balance helps protect the brain from oxidative damage and maintain its functional integrity

### Dysregulation of Oxidative Stress Modulation

Oxidative stress modulation is a biological mechanism caused by a disturbance in the normal balance between the production of free radicals (particularly reactive oxygen species) and antioxidant defenses; important to maintain the normal production of free radicals by detoxifying these reactive species, produced in several metabolic reactions that may be enhanced by the exposure to environmental stressors such as smoking or ultraviolet (UV) radiation.

The role of ROS in depression is well known. Studies reveal that depression is associated with lower intake of antioxidants such as vitamins A, C, and E, selenium and zinc, and B vitamins (B6, folate, and B12). Indeed, excessive ROS generation and lack of efficient antioxidant response triggers processes such as inflammation, neurodegeneration, tissue damage, and cell death. Thus, oxidative stress is correlated with the pathogenesis and progression of depression. Evidence suggests that high levels of brain lipidic peroxidation and other parameters such as nitric oxide and cyclooxygenase-2 (COX-2) activity, essential processes in the pathogenesis this disease, lead to high levels of oxidative stress. Together with the reduction of antioxidant defenses, these mechanisms highlight the role of oxidative stress as an essential player in the development and progression of depression. High levels of ROS essentially affect the other five pathways as they are associated with the stress response, neuroinflammation, neurotransmitter imbalance, and neurogenesis/synaptic plasticity imbalance that underlie the development and progression of depression.

Being a highly complex and multifactorial disease, other processes are involved in depression's pathogenesis. For example, hypoxia is involved in anxiety and depressive disorders, with few studies yet. Evidence points out that this process disrupts the neurohormonal homeostasis in the brain, increasing the potential for developing depression by promoting inflammation, apoptosis, dysregulation of serotonin pathways, and mitochondrial oxidative stress. Targeting hypoxia-related pathways is a promising tool for chronic anxiety and depressive disorders. Indeed, hypoxia contributes to high levels of oxidative stress, which is important in the context of depression. In fact, it has recently been found that hypoxia-ischemia induces an increase in ROS in neuron-like cells.

The main reactive oxygen species (ROS), superoxide radicals (O2⁻); hydrogen peroxide (H2O2), hydroxyl radicals (·OH), and singlet oxygen (1O2), are generated through processes such as immunity, apoptosis, protein phosphorylation, and other cellular signaling processes. Indeed, in the human body, ROS are mainly produced in mitochondria, peroxisomes, and the endoplasmic reticulum, being continuously generated by enzymatic reactions that involve cyclooxygenases, NADPH, and xanthine oxidases, and lipoxygenases, and through the Fenton reaction. At low levels, ROS are important for maintaining homeostasis and cellular processes.

However, when the production of these species increases in response to several stimuli such as pollutants and drugs, negative effects occur in cellular structures and processes. Indeed, proteins, lipids, nucleic acids, enzymes, cellular division, and cellular metabolism are highly affected and are associated with the development and progression of several diseases, such as cancer. Intense oxidant exposure affects unspecific targets, promoting an imbalance in adaptive pathways, such as nuclear factor-kB (NF-κB) and Nrf-2, which converge into pathological conditions.

To protect against the effects of increased ROS levels, cells have antioxidant defenses, such as superoxide dismutase (SOD) and catalase (CAT), which are essential for maintaining ROS homeostasis. Exogenous antioxidant defenses may also be introduced by diet or nutritional supplementation, such as carotenoids, vitamins C and E.

High-oxidative stress levels accentuate various cellular mechanisms implicated in MDD, including the stress response, neuroinflammation, neurotransmitter signaling disturbance, and impaired neurogenesis/synaptic plasticity.

The brain is vulnerable to oxidative stress due to its high oxygen consumption and lipid content. Also, neurons, astrocytes, and microglia are rich in mitochondria and NADPH oxidase (NOX), generating high levels of ROS. This makes oxidative stress an essential cause of neurodegeneration and a key factor in MDD. Addressing these changes with suitable antioxidants could be an effective strategy for treating MDD.

Depression is linked to reduced consumption of antioxidants like vitamins A, B, C, E, selenium, and zinc. There is also evidence that high levels of lipid peroxidation in the brain lead to increased oxidative stress levels. Analysis of lipid peroxidation markers in MDD (particularly malondialdehyde) demonstrated that this process was greater in MDD individuals than in controls and was associated with greater severity. When combined with a decrease in antioxidant defenses, these mechanisms support the role of oxidative stress as a key player in depression. Also, research reported elevated levels of peroxidation biomarkers combined with reduced antioxidant activity in the plasma and serum of individuals with MDD. Targeting hypoxia-related pathways may also be a promising tool for depressive disorders in the context of mitigating high-oxidative stress levels, enhanced by hypoxia. In fact, recent findings revealed that hypoxia-ischemia triggers ROS elevation in neuron-like cell lines. Treatment with edaravone enhanced cell viability and reduced ROS levels, likely due to its free radical-scavenging properties. Various studies have also observed reductions in antioxidant levels in both depressed patients and animal models of depression. Furthermore, it has been demonstrated that antidepressants have antioxidant properties, and antioxidants exhibit antidepressant effects. For instance, fluoxetine and citalopram enhanced SOD activity and ascorbic acid levels, and reduced malondialdehyde in MDD patients. Research conducted on cell lines further supports the link between oxidative stress and depression. Notably, the introduction of H₂O₂ to these cells resulted in a reduction in cellular viability. However, this effect was ameliorated when serotonergic compounds such as mirtazapine and L-TRP were applied. These compounds not only enhanced cell viability in the presence of H₂O₂ but also led to a reduction in ROS levels and mitigated DNA damage following exposure to oxidative stress.

These studies support the significant role of oxidative stress as an important factor in the pathogenesis of depression and suggest that antioxidant activity holds promise as a potential therapeutic approach.

Nutritional Modulators that Target Oxidative Stress Modulators

Studies support the link between oxidative stress and neurogenesis and synaptic plasticity impairments in depression, mainly by connecting them with BDNF. A recent study highlighted that the low levels of neurogenesis present in stress-induced depressed mice were rescued by upregulation of the mitochondrial antioxidant sirtuin 3, which is a promising strategy to confer stress resilience and improve depressed behavior. The administration of tilapia skin peptides to mice also improved depression-like behavior by regulating oxidative stress and neurogenesis. Indeed, the data obtained suggest that the BDNF/TRKB/CREB pathway may be involved in the antidepressant effects of these peptides, ameliorating neurogenesis and neural apoptosis. Additionally, this compound enhanced the Nrf2/HO-1 pathway, that regulates the expression of numerous antioxidant genes. The signaling pathway brain-derived neurotrophic factor (BDNF)-protein kinase B (Akt)/signal-related kinase 2 (ERK2) was also restored after administration of the flower essential oil of Tagetes minuta. In this study, oxidative stress was also mitigated, attenuating depressive-like behavior in mice. Another study revealed that melatonin increased antioxidant markers and increased neurogenesis in the hippocampus and pre-frontal cortex of rats previously treated with methotrexate. In fact, melatonin ameliorated the antioxidant defenses of the animals by improving the Nrf2 and BNDF expression. It also upregulated synaptic plasticity and enhanced the expression of doublecortin, an important marker of neurogenesis. The administration of the antioxidant carvedilol to depressed mice also increased brain glutathione and BDNF concentrations, and decreased MDA levels, presenting antidepressant-like effects. Similar results were observed with luteolin-7-O-glucuronide administration in mice. Indeed, this compound has been reported to exhibit antioxidant properties and to improve depression-like behavior, activating BDNF signaling and thus modulating neurogenesis and neuroplasticity. Rosmarinic acid also reversed LPS-induced depressive behaviors in mice by promoting the expression of the BDNF/Nrf2 pathway, also leading to the expression of antioxidant enzymes such as heme oxygenase-1 and NAD (P) H quinone dehydrogenase 1 (NQO1), downregulating the expression of proinflammatory genes. Both celastrol and thymoquinone also alleviated depressive and anxiety behaviors in rats by reversing the concentration of acetylcholine, dopamine, and serotonin, previously decreased by exposure to aluminum chloride. Additionally, these compounds increased BDNF expression and downregulated the oxidative-inflammatory markers (such as MDA and IL-6) in the brains of the rats. In SH-SY5Y cells, walnut polyphenols and the active compound urolithin A also improved oxidative damage caused by hydrogen peroxide, enhancing PKA/CREB/BDNF signaling and promoting neuroprotection.

Synaptic plasticity is a process that refers to the alteration of the strength or efficacy of synaptic transmission at existing synapses. Impairments in this mechanism contribute to several neuropsychiatric disorders, such as depression. BDNF is a neurotrophin involved in synaptic plasticity, extensively studied in the context of depression. Dysfunctions of this neurotrophin culminate in imbalances of synaptic plasticity and decreased excitatory neurons and glutamate, promoting depression. Indeed, levels of BDNF mRNA were reduced in postmortem samples collected from depressed brains (versus control) and as a result of suicide. Additionally, increased BDNF expression was found in the hippocampus in patients treated with antidepressants, compared with antidepressant-untreated patients. BDNF also plays a crucial role in hippocampal neurogenesis. Neurogenesis is the process of the formation of neurons de novo. In adults, it is known to occur in the lateral subventricular zone and in the dentate gyrus of the hippocampus. Several factors may affect this process. Indeed, in animal models, enhanced neurogenesis after regular physical exercise has been reported, whereas stress (acute or chronic) is known to decrease this process. Additionally, corticosteroids reduce neurogenesis and BDNF, other trophic factors (such as epidermal growth factor (EGF)), and 5-HT are known to enhance this process. Depression is also connected with impairment of hippocampal neurogenesis. Indeed, studies report low levels of typical biomarkers of neurogenesis, such as reduced volume of the dentate gyrus and reduced vascularization of the neurogenic niche. Additionally, after SSRI treatment, an increase in neurogenic hippocampal markers was observed in depressed individuals, such as neural progenitor cells in the dentate gyrus. Moreover, there are other situations where there is an association between depression and impairment of neurogenesis, for instance, it is known that Western diets are associated with a reduction in the neurogenic process and with an increased risk of depressive symptoms in adolescents.

### Role of Amino Acids in MDD

### L-Acetylcarnitine (LAC)

L-carnitine is an essential nutrient found in almost all tissues of the human body, including the brain. Intracellular carnitine levels are depleted under specific circumstances such as diabetes, hemodialysis, and carnitine deficiency secondary to genetic conditions that requires supplementation. LAC facilitates the transfer of activated long chain fatty acids (LCFA) through the carnitine shuttle, which is a series of reactions that transport fatty acids into the mitochondria, as acyl-carnitine ester, for β-oxidation. The carnitine shuttle prevents buildup of harmful LCFA and long chain acyl-coenzyme A (acyl-CoA). Carnitine also assists with transferring toxic compounds out of the mitochondria.

LAC is the acetyl derivative of L-carnitine with 2 carbons in the acyl moiety and is commonly found in plasma and body tissue. Carnitine, along with LAC, passes through the BBB and accumulates in the cerebral cortex, of which 10-15% is the LAC moiety. LAC is important for metabolic processes, including modulating glucose metabolism, stimulating glycogen synthesis, increasing plasma adenosine triphosphate (ATP) levels, and improving neurological function.

Although the exact mechanism of LAC for managing depressive symptoms is unclear, it has been hypothesized that its neuroplasticity effect, neurotransmitter regulation, and metabotropic glutamate (mGlu) receptor upregulation likely contribute. LAC has neuroprotective, anti-inflammatory, and antioxidant properties that also may improve depressive symptoms. In animal depression models, LAC rapidly improved depressive-like behaviors, restored glutamate levels, and increased type 2 mGlu (mGlu2) expression through epigenetic modification, specifically histone acetylation. In a recent study, lower LAC levels were reported in MDD patients compared with controls. Furthermore, lower LAC levels in patients with MDD were associated with greater severity and earlier onset of depressive symptoms. A significantly greater proportion of patients with treatment-resistant depression had a decrease in LAC levels (p=0.01), indicating a potential for augmenting antidepressants with LAC. A meta-analysis of studies investigating the effects of LAC on depressive symptoms showed that LAC significantly decreased depressive symptoms compared with placebo (standardized mean difference SMD −1.10; 95% confidence interval CI −1.65, −0.56; p<0.001). Furthermore, patients taking LAC had a comparable rate of adverse events (AEs) as those taking placebo/no interventions (odds ratio OR 0.86; 95% CI: 0.46, 1.63; p=0.648) and a significantly lower risk of AEs compared with antidepressants (OR: 0.21; 95% CI: 0.12, 0.36; p<0.001), with lower rates of gastrointestinal and nervous system AEs. Further investigation is needed to fully characterize the safety of LAC due to the limited data. Individuals who may benefit from LAC supplementation are likely those with lower LAC levels and elevated inflammatory markers.

### Alpha-Lipoic Acid (ALA)

ALA is a lipoamide that is synthesized from octanoic acid in the mitochondria and can be obtained through the diet. Once absorbed from the diet, ALA is reduced to dihydrolipoic acid (DHLA) and accumulates mainly in skeletal muscle, liver, and heart, and can cross the BBB. ALA plays an important role in mitochondrial energy metabolism by acting as a necessary cofactor for mitochondrial α-ketoacid dehydrogenase reactions. Additionally, ALA functions as an antioxidant by scavenging reactive oxygen species, chelating transition metals (e.g., iron and copper), and enhancing activity and synthesizing endogenous antioxidants or antioxidant enzymes. DHLA is among the most potent naturally occurring antioxidants and can regenerate other endogenous antioxidants, neutralize free radicals, and chelate metals that contribute to oxidative stress. ALA also significantly reduces cytokine-induced inflammation by decreasing production of IL-6, IL-1β, and TNF-α (p<0.05). Individuals receiving ALA 300 mg experienced a 15% significant reduction in IL-6 levels compared with placebo (p<0.001).

ALA has been investigated as augmentation therapy for MDD, given its biological properties and potential role in the pathophysiological factors involved in mood disorders. Augmenting desvenlafaxine with ALA in mice was associated with significantly greater improvement in depressive symptoms compared with either treatment alone, demonstrating a potentiating effect of these products together. Combination treatment of ALA and LAC also has been studied in MDD in light of their role in modulating mitochondrial function and metabolism, and neuroprotective effects; however, studies have reported inconsistent results. Together, LAC and ALA have been shown to reduce the number of damaged neuronal mitochondria and increase intact hippocampal mitochondria, thereby improving brain function. In preclinical studies, LAC and ALA reversed age-related increase in oxidants, reduced oxidative damage in the brain, and improved metabolic rate and physiological activity without causing further oxidation. Conversely, a randomized controlled trial of LAC plus ALA versus placebo in humans reported no significant differences in Montgomery-Åsberg Depression Rating Scale (MADRS) score between groups.

### N-Acetylcysteine (NAC)

NAC, the acetyl derivative of cysteine, is a glutathione precursor that is known as an antidote for paracetamol overdose. A critical role of NAC is restoring cellular glutathione concentrations by providing cysteine in glutathione production. The brain is susceptible to various reactive oxygen species that can cause oxidative cellular dysfunction; thus, oxidative stress is implicated in the pathogenesis of mood disorders. In addition to glutathione replenishment, NAC has been shown to have anti-inflammatory activity by reducing inflammatory cytokines in the brain, which is a potential mechanism for how NAC exerts antidepressant effects. NAC plays a role in neurotransmission by modulating glutamate pathways and regulating dopamine release.

Recently, NAC has emerged as a potential supplemental treatment for psychiatric and neurological disorders, including MDD. A significantly greater proportion of MDD patients receiving NAC responded to treatment and reached remission (p<0.05) in a study comparing adjunctive NAC with placebo. In a meta-analysis, NAC improved depressive symptoms and functionality compared with placebo over a follow-up of 12-24 weeks; however, NAC did not improve quality of life. In another meta-analysis of adjunctive NAC in MDD, there was no significant difference in efficacy between NAC and placebo, but individuals treated with adjunctive NAC, showed a positive trend towards efficacy, especially in those with higher MADRS scores. Notably, patients in the NAC group reported more gastrointestinal (33.9% vs. 18.4%; p=0.005) and musculoskeletal complaints compared with placebo (3.9% vs. 0%; p=0.025). Furthermore, adjunctive NAC has been demonstrated to improve symptom severity, function, and quality of life in MDD and major depressive episodes in bipolar disorder. Doses ranging from 1-3 g have been studied in patients with MDD. Doses of 2400-3000 mg/day have been found to be safe and effective for obsessive-compulsive and related disorders, NAC is generally well tolerated with no severe AEs reported in studies. The most common AEs associated with NAC were gastrointestinal, neurological, psychological, musculoskeletal, and dermatological. Individuals with glutathione deficiency may be potential candidates to supplement their current antidepressant treatment with NAC.

### L-Tryptophan

L-tryptophan is an essential amino acid used for protein synthesis and serotonin biosynthesis. Tryptophan undergoes degradation through the kynurenine pathway, where it is converted to kynurenine and ultimately serotonin. It has been suggested that an impairment of neuroprotective components of the kynurenine metabolic pathway plays a role in depression, as evidenced by lower tryptophan availability, higher tryptophan breakdown, and lower mean plasma kynurenic acid concentration in MDD patients. Alterations in tryptophan levels can impact serotonin synthesis and mood. An increase in HAM-D score was reported following acute tryptophan depletion in patients with a history of MDD. Another study also reported significantly increased HAM-D scores (p<0.0009) in medication-free remitted MDD patients. Low tryptophan levels and increased levels of its detrimental catabolites, kynurenine and quinolinic acid, in plasma are associated with the development of depressive disorders. Additionally, elevations in IL-6 in models of depressive symptoms are due to increased HPA activity, thereby increasing cortisol and activating tryptophan 2,3-dioxygenase (TDO), which produces more tryptophan catabolites and less serotonin. In the presence of inflammation, tryptophan produces kynurenic and quinolinic acid; therefore, it must be used with caution in patients with inflammation.

High L-tryptophan doses can result in mild nausea, tremors, dry mouth, and dizziness. Tryptophan should be used cautiously with monoamine oxidase inhibitors and SSRIs, which increase the risk of serotonin syndrome, which occurs when there is excessive synaptic serotonin in the brain. Serotonin syndrome typically presents with tremors, hyperreflexia, autonomic irregularities, and changes in mental status (e.g., agitation, restlessness, delirium, confusion). Other serotonin-elevating drugs include SSRIs, SNRIs, tricyclic antidepressants, St. John's wort, and pain medications. Despite the compelling evidence from tryptophan depletion studies that suggest that tryptophan is associated with depressive symptoms, the actual relationship between tryptophan and the pathophysiology of MDD has not been established. Furthermore, tryptophan must be used with caution when taken with medications that increase serotonin, a common mechanism of action of numerous standard-of-care treatments for MDD. In addition to being mindful of concomitant medications, prescribers and patients must consider the 2- to 3-times-daily dosing regimen, which may pose a compliance issue.

Zinc also has notable oxidative stress and anti-inflammatory effects in the brain, which contribute to its role in reducing depressive symptoms. Zinc supplementation has been shown to; reduce the expression of pro-inflammatory cytokines such as tumor necrosis factor-alpha (TNF-α) and interleukin-6 (IL-6) in microglial cells reducing cytokine levels which helps to mitigate neuroinflammation, a known contributor to depression; blocks the generation of ROS, which are harmful byproducts of cellular metabolism that contribute to oxidative stress and inflammation in the brain whereby reducing ROS levels, helps protect neuronal cells from damage and supports overall brain health; increases the expression of A20, a zinc-finger protein with anti-inflammatory properties, which inhibits the NF-κB signaling pathway involved in the production of inflammatory mediators and this upregulation helps to further reduce inflammation in the brain; inhibits astrogliosis, which is the excessive proliferation of astrocytes in response to injury or inflammation, this helps to maintain a healthier neural environment and supports neuroplasticity mechanisms; influences the signaling pathways that modulate brain-derived neurotrophic factor (BDNF), which is crucial for neuroplasticity and neuronal survival supporting the growth and differentiation of new neurons, beneficial for mood regulation and cognitive function.

Four Exemplary Nutritional Ingredients and MOA(s) within Process Pathway for the Composition:
1. L-methylfolate Increases nitric oxide production, enhancing antioxidant properties and reducing free radical production.
2. L-theanine: Reduces ROS and boosts antioxidant enzymes to mitigate oxidative stress
3. Vitamin D3 Modulates ROS signaling to help reduce the risk of oxidative stress
4. Zinc Regulates ROS scavenger enzymes to eliminate free radicals in the synapse

### Neuroinflammation Modulation

### Normal Anti-Inflammatory Processes

The body modulates neuroinflammation within the nervous system through a complex interplay of cellular and molecular mechanisms involving key mediators and receptors.

Microglia are the primary immune cells in the CNS and play a central role in neuroinflammation. Upon activation by injury or disease, microglia release pro-inflammatory cytokines and chemokines, which can exacerbate inflammation but also aid in tissue repair and debris clearance.

Astrocytes also contribute to neuroinflammation by releasing cytokines and modulating the blood-brain barrier's permeability, thus influencing the infiltration of peripheral immune cells.

Key mediators in neuroinflammation include cytokines (e.g., IL-1β, TNF-α), chemokines, and reactive oxygen species (ROS). These molecules can activate various signaling pathways, such as the NF-κB pathway, which further amplifies the inflammatory response.

Lipid mediators like arachidonic acid (AA) and docosahexaenoic acid (DHA) are also crucial. AA is metabolized into pro-inflammatory prostaglandins and leukotrienes, while DHA is converted into anti-inflammatory resolvins and neuroprotectins, which help resolve inflammation.

Receptors involved in modulating neuroinflammation include:
CD200-CD200R: This receptor-ligand pair, found on neurons and microglia, respectively, helps inhibit pro-inflammatory microglial activation, maintaining CNS homeostasis.
Dopamine receptors: These receptors, particularly the D2-like family, modulate neuroinflammation by influencing glial cell activity and the NLRP3 inflammasome.
   CB2 receptors: These cannabinoid receptors, although expressed at low levels in the CNS, play a protective role under neuroinflammatory conditions by modulating immune responses.

In summary, the body modulates neuroinflammation through the coordinated actions of microglia, astrocytes, cytokines, lipid mediators, and specific receptors like CD200-CD200R, dopamine receptors, and CB2 receptors. This intricate network ensures a balanced inflammatory response, crucial for CNS health and function.

### Dysregulation of Anti-Inflammatory Processes

### Inflammation and Inflammatory Biomarkers

Inflammation and depression are directly correlated and form a bidirectional loop that plays a critical role in the mechanism behind depression in a subgroup of MDD patients potentially causing downstream metabolic and behavioral effects. Increased inflammation causes the central nervous system (CNS) to elicit or intensify depressive symptoms such as negative mood, fatigue, anhedonia, increased pain sensitivity, an altered sleep pattern, and cognitive deficits. Depression can also promote inflammation by decreasing the sensitivity of the immune system to glucocorticoid hormones that stop the inflammatory response. Furthermore, in MDD, there is a lack of parasympathetic activity to counter the continual sympathetic activity, which results in elevated norepinephrine and epinephrine levels and low acetylcholine levels, which ultimately results in release of inflammatory mediators from immune cells.

The link between inflammation and depression lies in the cytokines, which elevate inflammatory signaling in the CNS, which subsequently leads to depressive symptoms.

Cytokines can activate indoleamine 2,3-dioxygenase (IDO), which converts tryptophan, a precursor of serotonin, into kynurenine, thereby decreasing serotonin production and availability in the brain. Other factors involved in the bidirectional pathway of inflammation and depression include psychological stressors, sensitization of cells to neurotoxic peptides, and oxidative and nitrosative stress. Cytokines can affect the production, metabolism, and transport of neurotransmitters responsible for mood (i.e., dopamine, norepinephrine, serotonin, and glutamate). Additionally, cytokines may lead to a decrease in GABA release, which can further exacerbate inflammation in the CNS. Elevated levels of proinflammatory cytokines in the CNS may deleteriously influence neurotransmitters that are central to depression pathophysiology by increasing the activity of transporters that clear monoamine neurotransmitters from neuronal synapses, by decreasing the synthesis of monoamines, and by increasing excitatory and potentially neurotoxic glutamate activity through N-methyl-D-aspartate (NMDA) receptor activation and reduced astrocytic reuptake. Moreover, they are known to decrease neuroplasticity and cause oxidative stress by generating nitrogen and oxygen radicals, which can promote oxidative neurotoxicity. Serologic markers of systemic inflammation are potentially useful tools that may inform clinicians about optimal treatment paradigms and antidepressant selection for individual patients. Antidepressants have been shown to affect the immune system and levels of proinflammatory cytokines.

### Oxidative Stress and Depression's Associated Neuroinflammation

Several studies have connected inflammation with depression. Indeed, elevated levels of immune markers, including granulocytes, monocytes, tumor necrosis factor (TNF), interleukin-6 (IL-6), and microglial activation, are observed in depressed individuals. Another study revealed that the mRNA levels of proinflammatory markers (IL-1β, IL-6, TNF-α, and lymphotoxin A) and anti-inflammatory markers (cytokine IL-10, and of IL-1 receptor antagonist (IL-1RA)) were substantially increased and decreased, respectively, in the prefrontal cortex of depressed individuals who committed suicide. Antidepressants such as imipramine also reduced microglial activation, decreasing proinflammatory cytokine levels, and reversing stress-induced social avoidance in mice. High levels of production of ROS in mitochondria are highly connected with inflammation, promoting oxidative stress, an important player in the pathophysiology of depression. Indeed, several oxidative stress markers have been found to be increased in alcohol-induced aggressive and suicidal behaviors. Thus, the immune system influences neuronal networks involved in depression, playing an essential role in the pathogenesis of this disease.

Increased levels of oxidative stress generate dysregulation of the inflammatory response. Modifications of cell signaling promoted by oxidative stress leads to enhanced production of proinflammatory factors, promoting a proinflammatory response (particularly neuroinflammation, in the central nervous system), that is also modulated by oxidative stress. By activating inflammatory pathways via nuclear factor-KB and the mitogen-activated protein kinase family of stress kinases, ROS are crucial for cell signaling. When present in excess, they promote cell damage and formation of proinflammatory molecules, such as malondialdehyde, ultimately leading to cell death. An overactivated inflammatory system and increased levels of ROS act synergistically, promoting the onset and development of depression.

Several studies demonstrate this connection, the administration of the anti-inflammatory compound muscone to mice ameliorated depression-like behavior by regulating inflammatory responses and improving oxidative stress markers, particularly malondialdehyde (MDA), SOD, and GPx. In another study, the administration of apple phenolic extracts against lead acetate (Pb(Ac)2)-induced cognitive impairment and depression/anxiety-like behavior in mice revealed that the increased cellular oxidative damage and the levels of proinflammatory cytokines interleukin (IL)-1β, IL-6, and tumor necrosis factor-α were attenuated after the administration of apple phenolic extracts via the regulation of oxidative stress, neuroinflammation, and apoptosis via the miR-22-3p/Sirtuin 1 (SIRT1) signaling pathway. Another similar study with cinnamic acid also improved lipopolysaccharide-induced depressive-like behaviors in mice by inhibiting neuroinflammation and oxidative stress. Indeed, proinflammatory cytokines (IL-6 and TNF-α) and oxidative stress markers (SOD, glutathione, and MDA) in the hippocampus and cortex of depressed mice were significantly improved by cinnamic acid administration. MDA is an agent that leads to protein damage and generation of advanced lipoxidation products with proinflammatory characteristics, detected in patients with depression. Another recent study revealed that curcumin attenuated lipopolysaccharide-induced anxiety/depression-like behaviors in rats by decreasing cerebral oxidative stress. This compound increased the activity of SOD and GPx enzymes and reduced MDA concentration. Additionally, it exerted anti-inflammatory properties by inhibiting microglial activation. p-Coumaric acid is a compound that has a protective role against inflammation and oxidative stress (by scavenging ROS) in various diseases. Another recent study revealed that this acid reverses depression-like behavior through inhibition of glycation end products (AGEs) and receptor for AGE (RAGE), AGE-RAGE-mediated neuroinflammation. The administration of quercetin to zebrafish also alleviated oxidative stress and neuroinflammation induced by lipopolysaccharide (LPS), ameliorating behavioral abnormalities. Proinflammatory compounds such as TNF-α and IL-1β decreased and the antioxidant glutathione increased, versus LPS-treated fish. Recently, targeting neuroinflammation using polyphenols was described as a promising therapeutic against inflammation-associated depression. Indeed, polyphenols are plant-derived natural compounds with strong antioxidant properties. In another study, these compounds were also shown to inhibit MAPK signaling pathway-mediated oxidative stress and inflammation in depression. Similar results were obtained with LQFM212, 2,6-di-tert-butyl-4-((4-(2-hydroxyethyl) piperazin-1-yl)methyl) phenol, a piperazine derivative. This compound exhibited elevated antioxidant effects and also ameliorated LPS-induced behavioral, inflammatory, and oxidative changes in the tested animals. Another study in humans aimed to investigate the levels of NOX1 and raftlin in depressed patients. NOX1 is an important source of ROS, and raftlin is important in inflammatory processes. The study concluded that depressed patients had increased serum NOX1 and raftlin levels compared to controls, highlighting the implication of oxidative stress and inflammatory processes in depression. Resveratrol has also been linked to antioxidant, anti-inflammatory, and antidepressant effects. A study revealed that this compound attenuated the depressive-like behavior in stressed animals, mediating alteration in the hippocampal levels of anti-inflammatory and proinflammatory cytokines and inducing antioxidant actions, such as the modulation of SOD and CAT activities.

In another study, the flower essential oil of Tagetes minuta also attenuated oxidative stress and restored the cellular pathway BDNF-Akt/ERK2, leading to an attenuation in inflammation and depressive-like behavior in mice. The oxidative stress induced by indoor air pollution from solid fuel use also markedly increased inflammation, promoting depression and cognitive function impairment in middle-aged and older Chinese adults. Another study also revealed that astilbin, that has antioxidant characteristics, ameliorated depressive-like behavior by regulating astrocyte-mediated neuroinflammation, caused by postnatal immune activation. A study in humans also concluded that the frequently observed post viral chronic fatigue and affective features after coronavirus disease (COVID) are related to high levels of inflammation, oxidative damage, and lowered antioxidant defenses. Depressive symptoms in COVID-19 survivors were also associated with high levels of inflammation, that correlated with low levels of glutathione in the anterior cingulate cortex. Vitamin E, an antioxidant compound, has also been reported to be beneficial for the oxidative stress and inflammation of depressive patients.

All this evidence supports the connection between the inflammatory processes present in depression and oxidative stress. Altogether, these mechanisms promote the development and onset of depression, being important therapeutic targets.

Nutritional Modulators that Target Neuroinflammation Processes

The inflammatory system has been implicated in the onset, phenomenology, and comorbidity of mood disorders. In particular, inflammation can alter mood, energy, sleep, cognition, and motivation, which are key aspects of MDD. Neuroinflammation has been extensively studied in mood disorders and has been implicated in depressive symptoms and neurodegeneration, two commonly comorbid conditions. Inflammation induces neurodegenerative processes, as evidenced by neuronal and glial cell atrophy/loss, in addition to reducing neuroprotection and neuronal repair. It has been suggested that a mechanistic link between inflammation and depression is the impact of cytokines on serotonin levels, glutamate metabolism, the dopamine pathway, the hypothalamic-pituitary-adrenal (HPA) axis, microglial activation, and brain structure. Increased levels of C-reactive protein (CRP]; ≥1.0 mg/L), interleukin-6, interleukin-1, and tumor necrosis factor-α (TNF-α) have been repeatedly shown in clinical trials and meta-analyses to have a positive correlation with MDD. Furthermore, cytokines may interfere with oligodendrocytes, which modulate glutamate transmission, by contributing to glutamate excitotoxicity and axonal damage in the white matter of the brain. This cascade of events ultimately impacts neural plasticity through excitotoxicity, decreased neurogenesis, increased glutamatergic activation, oxidative stress, and induction of apoptosis. These inflammation-mediated changes may have implications for the long-term course of MDD, including response to treatment, so managing inflammation is likely an important aspect of treating MDD. In particular, levels of IL-6 and CRP have been associated with an increased likelihood of experiencing treatment-resistant depression, suggesting that there is a role for anti-inflammatory interventions to be used as adjunctive treatments for patients with MDD.

More recently, epigenetic mechanisms have been identified in the pathophysiology of depressive symptoms and potentially increase the risk for developing MDD. Epigenetic factors are inherited and acquired mechanisms that regulate gene function, by altering DNA methylation and chromatin structure, without modifying DNA sequence. Nucleosomes, the basic units of chromatin, are formed by wrapping DNA around histone octamers, which can be modified via acetylation to increase gene expression or methylation to activate or repress gene transcription, depending on the amino acid involved. These epigenetic modifications to DNA and histones often occur secondary to stress and may result in downstream effects that exaggerate or reduce depression-like behavior. When considering management approaches to target the different mechanisms behind MDD, there is an opportunity to incorporate established medical foods and supplements and also explore the use of additional products in certain subsets of patients.

### Vitamin D

Vitamin D plays a critical role in several physiologic processes, such as muscle function, regulating cell growth, cancer prevention, metabolic signaling, inflammation, and autoimmunity. Vitamin D is integral in a number of brain processes including neuroimmunomodulation, neuroplasticity, neuroprotection, and brain development, which suggests its potential link to depressive disorders. It is thought that vitamin D may affect brain function by acting on vitamin D receptors (VDRs) located in the CNS. VDRs are found in various regions of the brain involved in depressive symptoms including the hypothalamus, prefrontal cortex, hippocampus, thalamus, and substantia nigra. VDR gene polymorphisms have been shown to be associated with cognitive and behavioral impairment, and increased anxiety. Potential actions of vitamin D in the brain include neurotrophin stimulation, antioxidation, and anti-inflammation by inhibiting the release of cytokines and metalloproteinases. Additionally, vitamin D promotes glutathione metabolism in neurons, providing protection from oxidative degeneration.

Vitamin D deficiency has been shown to be associated with the presence of mood disorders and reduced cognitive functioning. In a cross-sectional study, patients with serum 25-hydroxyvitamin D levels <10 ng/mL had a significantly greater likelihood of developing a mood disorder (OR: 11.7; 95% CI 2.04, 66.9) than those with adequate vitamin D levels. Additional support is provided by a meta-analysis that demonstrated that low vitamin D levels are associated with a significantly increased risk for depressive symptoms (HR 2.2; 95% CI 1.4, 3.5; p<0.001). Patients with vitamin D deficiencies not only have a higher risk of depression, but also have greater duration and severity of depressive symptoms.

Numerous studies have demonstrated the effectiveness of vitamin D supplementation in improving depressive symptoms. In a meta-analysis of randomized controlled trials evaluating the effect of vitamin D supplementation (≥800 IU daily) on depressive symptoms, 10 of 15 studies reported significant improvement. When the studies were analyzed to exclude those with biological flaws, there was a significant effect size (SMD: 0.78).

Vitamin D toxicity rarely occurs but is caused by consuming excessively high doses. Doses >50,000-100,000 IU/day can cause hypercalcemia and hyperphosphatemia. Currently, the tolerable upper intake level of vitamin D is 2000 IU/day; however, doses up to 4000 IU/day have been shown to carry low risk of hypercalcemia. Vitamin D toxicity typically manifests as nausea, dehydration, and lethargy. Doses of vitamin D 400-18,400 IU/day have been studied in depression.

Although the exact mechanism behind vitamin D deficiency in depressive disorders is not clear, supplementing vitamin D carries low risk, given the overall health benefits of vitamin D and low toxicity at doses of 1000-2000 I U. Experts recommend using 10,000 IU, then retesting and adjusting in order to reach adequate vitamin D levels.

### Omega-3 Fatty Acids

Omega-3 fatty acids are recognized for their multitude of health benefits due to their anti-inflammatory, antiarrhythmic, antithrombotic, and hypolipidemic effects. Omega-3 polyunsaturated fatty acids (PUFAs) can only be obtained through the diet and are synthesized by consuming short-chained omega-3 fatty acids, which produce eicosapentaenoic acid (EPA) and docosahexaenoic acid (DHA). EPA and DHA are involved with different processes related to brain function. DHA is responsible for maintaining the structural integrity of the phospholipid in neuronal cell membranes. Low levels of DHA cause abnormalities in the brain that impact neuron size, nerve growth factor levels, auditory and olfactory responses, and learning and memory. EPA has important physiological functions such as modulating cytokines that affect neurotransmission and neuromodulation. EPA also is thought to reduce inflammation by decreasing IL-1 and TNF-α levels, and also inhibits the upstream mitogen activated protein kinase (MAPK) pathway.

Omega-3 PUFAs are thought to improve depression through its role in the uptake, release, metabolism, and receptor function of serotonergic and dopaminergic transmission.

Additionally, the anti-inflammatory actions of omega-3 PUFAs are an important mechanism that may address depression-related inflammation. Omega-3 PUFAs, including DHA and EPA, have been found to modulate and reduce neuroinflammation. In a rodent study, omega-3 PUFA deficiency was correlated with increases in the proinflammatory cytokines IL-6 and TNF-α, and CRP. In humans, omega-3 PUFAs also were found to be lower in patients with depressive symptoms compared with non-depressed individuals. In a study conducted by Rapaport et al., MDD patients who had high levels of inflammation (as measured by hs-CRP, IL-1RA and IL-6), experienced greater improvement with EPA than placebo or DHA, while those who received DHA experienced less improvement than placebo. Patients without any high inflammatory biomarkers experienced a decreased response to EPA than those receiving placebo or DHA. By week 8, patients with high biomarkers who received EPA had at least an 11-point decrease in HAM-D scores, compared to those who received placebo who were progressively less responsive and had increases in inflammatory biomarkers. Despite these positive findings, in a recent meta-analysis, supplementation with omega-3 PUFAs showed little to no effect on risk of depression or anxiety symptoms (number needed to harm, 1000), and inconclusive findings on its effects on depression symptom severity and risk of remission. Patients with increased inflammation and comorbid inflammatory diseases may benefit from omega-3 PUFAs; however, there is conflicting evidence for its use in depressive disorders.

### Coenzyme Q10 (CoQ10)

CoQ10, also known as ubiquinone, is a potent antioxidant that possesses anti-inflammatory and neuroprotective properties. It protects cells from reactive oxygen and nitrogen species by regenerating oxidized tocopherol and ascorbate, and also enhances mitochondrial activity in the brain. CoQ10 also is involved in a number of biological roles such as cellular membrane repair, regulation of inflammation, and gene expression. As such, reduced levels of CoQ10 in the body are associated with increased free radicals and free radical damage, along with decreased mitochondrial energy production. Patients with MDD are associated with significantly lower plasma CoQ10 levels than healthy individuals. Furthermore, patients with treatment-resistant depression were found to have significantly lower CoQ10 levels than those who were not treatment resistant. In patients with bipolar disorder, supplementing their psychotropic medications with CoQ10 resulted in a significant decrease in MADRS score from baseline after 4 weeks, with significant changes seen as early as 2 weeks. CoQ10 is generally well tolerated and associated with minimal severe AEs. Clinicians should consider supplementing antidepressant regimens with CoQ10 in patients with treatment-resistant depression.

Four nutritional ingredients and MOA(s) within process pathway for the composition:
(a) L-methylfolate lowers inflammation by regulating pro-inflammatory markers through one-carbon & methylation cycles;
(b) Vitamin D3 Modulates the immune system to reduce pro-inflammatory cytokine production;
(c) L-theanine inhibits nuclear factor kappa B (NF-κB) pathway, thereby reducing expression of proinflammatory factors, cyclooxygenase-2 (COX-2), prostaglandin E2, inducible nitric oxide synthase, as well as NO.

### Neurotrophic and Synaptic Plasticity Modulation

### Normal Neurotrophic and Synaptic Plasticity Processes

The body modulates neuroplasticity within the nervous system through various mechanisms involving key mediators and receptors.

Brain-derived neurotrophic factor (BDNF) is a central mediator of neuroplasticity. BDNF promotes the formation, stabilization, and potentiation of synapses through its high-affinity TrkB receptors. Neuronal activity regulates the synthesis and release of BDNF, which in turn enhances synaptic plasticity and is crucial for learning and memory.

Dopamine is another critical neuromodulator that influences neuroplasticity, particularly through its interaction with NMDA receptors. Dopamine modulates NMDA receptor activity via D1 and D2 receptor subtypes, which are essential for long-term potentiation (LTP) and synaptic plasticity.

Glial cells, including astrocytes and microglia, also play significant roles in neuroplasticity. Astrocytes regulate synaptogenesis and maintain the balance between excitation and inhibition in neuronal networks, while microglia continuously monitor and sculpt synapses, facilitating the remodeling of brain circuits.

Neuregulins (NRGs) and their ErbB receptors are involved in synaptic plasticity by modulating neuronal excitability and neurotransmission. NRGs/ErbB signaling affects various forms of synaptic plasticity, including LTP and long-term depression (LTD), and is essential for proper brain function and cognitive processes.

Serotonin (5-HT) also modulates synaptic plasticity, particularly in the hippocampus, amygdala, and prefrontal cortex. Serotonin influences synaptic reorganization and is involved in cognitive and emotional functions.

In summary, the body modulates neuroplasticity through the actions of BDNF, dopamine, glial cells, neuregulins, and serotonin, along with their respective receptors such as TrkB, NMDA, D1/D2, ErbB, and 5-HT receptors. These mediators and receptors are crucial for the dynamic regulation of synaptic connections and overall brain function.

### Dysregulation of Neurotrophic and Synaptic Plasticity

Civilizational development, advances in medicine and lifestyle changes, including dietary changes, contribute to the constant improvement of the quality of human life. On the other hand, the growing number of seniors results in an increasing percentage of the population suffering from miscellaneous central nervous system (CNS) diseases. Despite significant progress being made in neurobiology over recent years, it is still not possible to precisely define the mechanism leading to nervous tissue pathology. Serious difficulties in developing effective drugs result from limited bioavailability, depending on their ability to cross the blood-brain barrier (BBB), as well as their rapid degradation, leading to the need to administer high concentrations of substances, which indicates the need to search for new therapeutic solutions.

The regenerative processes of the brain tissue are characterized by a limited scope and are regulated by the properties of the tissue environment, dependent on changes in the physiology of the body. The brain's ability to create structural and functional adaptive changes is known as brain plasticity. This process includes neurogenesis, synaptogenesis, and neurochemical changes of the CNS. Therefore, the regulation of neuroplasticity processes raises high clinical hopes, and its forms include pharmacotherapy and biological therapy, causing a synergistic effect between neurogenesis and synaptogenesis. Physical activity, including properly conducted rehabilitation, is an important treatment option inducing neurogenesis, strongly correlating with the improvement of the memory and attention of patients. High-intensity exercise has been shown to increase neurogenesis in the hippocampus, while medium and low-intensity exercise improves the maturation and survival of newly formed neurons. Moreover, the plasticity processes are also influenced by therapeutic methods based on physical stimuli, e.g., therapeutic hypothermia, deep brain stimulation (DBS) and transcranial magnetic stimulation (TMS).

The compensatory plasticity occurring in a damaged brain is an entirely different process than plasticity in a healthy brain. This process begins in critical conditions related to inflammation, edema, the degeneration of nerve fibers, apoptosis, and metabolic disorders. Synaptogenesis is based on enhancing existing synaptic pathways and then forming new connections. The remaining but diminished boutons are activated, other subcortical or cortical structures take over the function of the impaired brain region, and as a result, the functions of the damaged area may be restored.

The most critical signaling pathways involved in brain plasticity include the phosphoinositide-3-kinase-protein kinase B/protein kinase B (PI3K/Akt), phospholipase C/inositol trisphosphate/Ca2+/calmodulin-dependent protein kinase II (PLC/IP3/CAMKII), and mitogen-activated protein kinase/extracellular signal-regulated kinase 1/2 (MAPK/Erk) pathways, which are activated by multiple growth factors: brain-derived neurotrophic factor (BDNF), insulin-like growth factor 1 (IGF1), nerve growth factor (NGF), fibroblast growth factor (FGF), and Wnt.

Brain plasticity processes are indissolubly associated with PI3K pathway activation. The activation of PI3K results in the generation of phosphatidylinositol 3,4,5-trisphosphate, which stimulates the protein kinase Akt/protein kinase B (PKB). Akt kinase plays an essential role in controlling the functionality of many proteins involved in the regulation of cell survival. Among others, Akt, by death, phosphorylating the Bcl-2-associated death promoter (BAD), inhibits apoptosis. In turn, Akt phosphorylates the nuclear factor kappa B inhibitor (IκB), leading to its degradation and NFκB activation, thereby increasing neuronal survival. In addition, Akt limits apoptosis by phosphorylating the transcription factor forkhead 1 (FKHRL1), regulating the expression of apoptosis-promoting gene products such as the Fas ligand (FasL), and by negatively regulating glycogen synthase 3β kinase (GSK-3β), which supports apoptosis. Ras induces activation of PI3K. Ras activation of PI3K is the most important signaling pathway responsible for the survival of neurons. In parts of the neurons, the Ras-dependent activation of PI3K is the major survival-promoting pathway, induced by neurotrophins. However, Ras-independent PI3K signaling may also occur. PI3K binds to the Grb2-associated-binding protein 1 (Gab-1) activated by phosphorylated Grb-2. The adapter protein Gab-1 and the formation of the complex facilitate the activation of PI-3 kinase.

Another important component of brain plasticity is neurogenesis, which is regulated by microenvironmental factors, neurotrophins, neurotransmitters, growth factors and hormones. It has been shown that in certain brain regions, the differentiation of neuronal stem cells (NSCs) present in a fully developed brain into mature neurons occurs throughout life. However, the reduction in the rate of this process, as well as the survival capacity of the newly formed nerve cells, correlates with age. The strongest neurogenesis occurs mainly in the subgranular zone (SGZ) and the subventricular zone (SVZ), in the areas of the brain responsible for memory, learning, and olfactory sensation.

Focusing the therapy on supporting neuroplastic processes seems to be a promising treatment strategy. One of its elements may be the use of natural substances that can be used both in neuroprotection and in supporting conventional treatment.

Neural plasticity is important in several aspects of human physiology, and its expression is observed in both the central nervous system (CNS) and the peripheral nervous system (PNS). Neurons are the principal producers of BDNF, which begins as preproBDNF and then is processed into a preprotein called proBDNF. ProBDNF undergoes further processing to form the mature form. BDNF produces dimers, and biologically active BDNF is typically made up of a dimer composed of two identical mature peptide chains linked together by noncovalent interactions.

The human BDNF gene contains 11 exons (I-IX, Vh, and VIIIh), and the production of BDNF transcripts is cell and activity specific, with different transcripts playing different roles in biochemical processes. Exon IV is the most extensively studied because it plays a role in the modulation of mood, cognition, and behavior. The methylation status of the promoter associated with this exon has emerged as a potential biomarker for antidepressant therapy in individuals with MDD.

BDNF plays an important role in many processes, including neuronal survival and differentiation, synaptic plasticity, neurogenesis, neuroprotection, learning, memory, mood control, and other cognitive processes. Low BDNF levels relate to brain shrinkage, cognitive decline, and an increased risk of mental illnesses. BDNF also influences the release and activity of different neurotransmitters, affecting neuronal communication and general brain function. This neurotrophin is also involved in the formation of dendrites and synaptic specializations, maturation and refinement of dendritic arbors, and axon growth and differentiation. Based on this neurotrophin's actions, several therapies and techniques have been shown to boost BDNF levels, including physical exercise, omega-3 fatty acid supplementation, and several antidepressants, such as SSRIs.

The relationship between BDNF and MDD is a large topic of investigation. The neurotrophic theory of depression is based on the relationship between lower BDNF levels and an increased risk of depression.

Postmortem examinations of brains from patients with depression, including suicide cases, have revealed reduced BDNF mRNA levels, confirming this trend. Animal models of depression also aligned with this evidence, showing that chronic stress and depression lead to diminished BDNF levels, increased cell death, and reduced neurogenesis in the hippocampus, alongside decreased BDNF expression in other brain areas. A genetic variation known as Val66Met, which naturally occurs in the BDNF gene, is also linked with MDD.

Nutritional Modulators that Target Neurogenesis/Synaptic Plasticity Processes

Neuroplasticity is a complex physiological process occurring in the brain throughout its entire life. However, it is particularly important in the case of central nervous system (CNS) disorders. Neurological recovery largely depends on the ability to reestablish the structural and functional organization of neurovascular networks, which must be pharmacologically supported. For this reason, new forms of therapy are constantly being sought. Including adjuvant therapies in standard treatment may support the enhancement of repair processes and restore impaired brain functions. The common hallmark of nerve tissue damage is increased by oxidative stress and inflammation. Thus, studies on flavonoids with strong antioxidant and anti-inflammatory properties, with potential applications in neurointervention, have been carried out for a long time. However, recent results have revealed another important property of these compounds in CNS therapy. Flavonoids possess neuroprotective activity, and promote synaptogenesis and neurogenesis, by, among other means, inhibiting oxidative stress and neuroinflammation.

### Neuroplasticity Modulators

Several research works demonstrate the connection between oxidative stress and serotonin in the context of depression. A recent study showed that seleniummodified interactions with pathways such as tryptophan/kynurenine. Indeed, tryptophan may be metabolized to kynurenine through proinflammatory cytokines and directly by ROS. This molecule may be further metabolized to prooxidant compounds, 3-hydroxykynurenine and quinolinic acid, associated with the pathogenesis of depression.

Several research works demonstrate the connection between oxidative stress and serotonin in the context of depression. A recent study demonstrated that selenium-modified fluoxetine derivatives simultaneously target 5-HT reuptake (antidepressant action) and oxidative stress, leading to promising results. Additionally, another study revealed that mirtazapine and L-tryptophan (both involved in serotonergic pathways) counteracted hydrogen peroxide-induced cellular stress, highlighting the role of serotonergic pathways in oxidative stress. Another study revealed that saffron intake also protected human neurons from oxidative stress, stimulating the production of dopamine, 5-HT, and BDNF. In addition, saffron inhibited the expression of the 5-HT transporter (SERT). Indeed, all these data provided new insights into the context of oxidative stress related to depression. Other data also revealed that oxidative DNA and RNA damage was attenuated after the treatment with SSRIs, in the case of unipolar depression. Nacre extract from pearl oyster also suppressed LPS-induced depression and anxiety in mice. The high levels of oxidative stress induced by LPS were accompanied by changes in 5-HT receptors (5-HT1A and 5-HT2A) and BDNF, suggesting an antidepressant action. Persimmon leaf extract is also associated with antioxidant properties. The administration of this extract to depressed mice prevented dendritic spine loss through the inhibition of 5-HT reuptake, increasing 5-HT brain levels, alleviating the depressive-like behavior of the animals. Another study supported the connection between 5-HT and oxidative stress. This study in murine RAW264.7 macrophages revealed that 5-HT and its metabolites reduced oxidative stress and prevented the production of inflammatory cytokines by macrophages. In a postpartum depression rat model, supplementation with the probiotic Lactobacillus casei improved depression-like behaviors. Indeed, this probiotic reversed rats' gut microbiota, leading to several processes, including enhanced monoamine expression and activation of the BDNF/ERK1/2 pathway, and decreased oxidative stress (suppressing the increase in MDA and promoting SOD activity).

Altogether, these studies highlight the connection between the 5-HT imbalance present in depression and oxidative stress.

Flavonoids: Found in various fruits, vegetables, and teas, flavonoids promote neuroplasticity by inhibiting oxidative stress and neuroinflammation. They also enhance synaptogenesis and neurogenesis through the activation of signaling pathways such as BDNF/CREB.

Polyphenols: These compounds, present in foods like berries, tea, and red wine, improve neuroplasticity by crossing the blood-brain barrier, reducing oxidative stress, and modulating neurotrophic factors. They enhance synaptic plasticity and cognitive functions by activating pathways like Wnt signaling.

Curcumin: The active component of turmeric, curcumin, enhances neuroplasticity by modulating inflammatory pathways and increasing levels of brain-derived neurotrophic factor (BDNF), which supports neuronal growth and differentiation.

Ginseng (Panax ginseng): Ginseng has been shown to improve neuroplasticity by modulating the PI3K/Akt and BDNF/CREB pathways, which are involved in neuronal survival and synaptic plasticity.

Resveratrol: Found in grapes and red wine, resveratrol enhances neuroplasticity by activating SIRT1 and AMPK pathways, which promote neuronal survival and synaptic plasticity.

Bacopa monniera: This herb, commonly used in traditional medicine, enhances neuroplasticity by increasing synaptic density and modulating neurotransmitter levels, thereby improving cognitive functions.

Saffron extract (Protected human neurons from oxidative stress, stimulating the production of dopamine, 5-HT, and BDNF,

Persimmon leaf extract prevented dendritic spine loss, alleviating the depressive-like behavior.

Four Nutritional Ingredients and MOA(s) within the Process Pathway for the Composition:
1. L-methylfolate: Improves neuroplasticity through normalization of monoamine production.
2 Zinc Bisglycinate: Functions as a neuromodulator, regulating neurogenesis and communication through various membrane receptors (NMDA), ion channels and transporters.
3. L-theanine has been shown to influence production of BDNF, as well as NMDA receptors involved in neuroplasticity.
4. Vitamin D3: Numerous studies have demonstrated that vitamin D is a potent regulator of the production of neurotrophic substances, such as BDNF, a neurotrophin (NT)-3, and nerve growth factor (NGF).

### HPA Stress Response Modulation

### Normal HPA Stress Response

The brain is the central organ of stress and adaptation to social and physical stressors because it determines what is threatening, stores memories, and regulates the physiological as well as behavioral responses that may be damaging or protective. The physiological responses that produce adaptation via “allostasis,” include not only the hypothalamic-pituitary-adrenal (HPA) axis and the autonomic nervous system, but also their non-linear interactions with the metabolic system and the pro- and anti-inflammatory components of the immune defense system. Exposure to multiple stressors and dysregulation of nonlinear interactions (e.g., inefficient on/off responses) lead to wear and tear on the body and brain, termed allostatic load and overload.

Allostasis is the active process of adapting to stressors via mediators such as cortisol and the autonomic, metabolic and immune system that act together in a non-linear fashion to maintain homeostasis. Allostatic load refers to the cumulative effect of multiple stressors as well as the dysregulation of the non-linear network of allostasis (eg, too much or too little cortisol, or adrenaline or inflammation in response to a challenge). Allostatic overload refers to the cumulative pathophysiology that can result from dysregulation and excessive stress. Allostasis and allostatic load/overload are more precise biological concepts than the word “stress” to describe adaptation and maladaptation to “stressors.” They include the physiological effects of health-promoting and health-damaging behaviors as well as stressful experiences, health behaviors (i.e., smoking, alcohol, poor diet, or lack of sleep), resulting from the experience of stress, can also play a role and contribute to allostatic load/overload.

“Stress” can be divided into “good stress”, “tolerable stress”, and “toxic stress”. Early life stress can alter neural architecture to increase adverse reactions to stressors, leading to toxic stress. “Biological embedding” of these effects during critical or sensitive periods of early development has lasting effects through the life course. Among the most important early life experiences are those that involve abuse and neglect, on the one hand, versus the establishment of a strong, positive attachment of the child to the caregiver; these alter the ability of the individual to engage in cooperative social experiences or feel excluded and hostile to the social environment later in life.

The brain is a target of stressful experiences, and glucocorticoids, along with excitatory amino acid neurotransmitters, alter neuronal architecture by causing dendritic retraction or expansion and decreased or increased synapse density, depending on the brain region, and by inhibiting dentate gyrus neurogenesis. Many intra- and intercellular mediators and processes are involved in changing the brain during stress and recovery from stressful experiences.

It does so by focusing primarily on three brain regions, the hippocampus, amygdala and prefrontal cortex (PFC), in full recognition of the fact that stress has widespread effects throughout the brain. This review also emphasizes the complex, non-linear interactions among different stress mediators that are central to the concepts of allostasis and allostatic load/overload, in which non-linearity applies not only to systemic hormones but also to intra- and extracellular mediators in the brain. Because of this, the many changes caused by stress often result in an inverted U dose-response relationship.

### Dysregulation of Neurotrophic and Synaptic Processes

### Unraveling the Hypothalamic-Pituitary—Adrenal Axis Dysregulation in Major Depressive Disorder

HPA axis is important in regulating functions such as energy balance, reproduction, and response to stress. The elements of the HPA axis are the hypothalamus, pituitary gland, and adrenal glands. The hippocampus contains neuroendocrine neurons that synthesize and secrete vasopressin and corticotropin-releasing hormone (CRH), mainly in the paraventricular nucleus (PVN). In response to stress, CRH and vasopressin are released into hypophysial portal vessels that access the anterior lobe of the pituitary gland to secrete adrenocorticotropic hormone (ACTH) via the activation of the cyclic adenosine monophosphate (cAMP) pathway, after binding to the CRH receptor 1 and vasopressin V1b (or V3) receptor, respectively. The adrenal cortex produces glucocorticoid hormones (mainly cortisol in humans) in response to stimulation by ACTH, which binds to the melanocortin type 2 receptor. These glucocorticoids regulate physiological responses and inhibit further HPA axis activation.

When the HPA axis is triggered, it leads to an increase in glucocorticoid levels, an important component in the body's adaptation to stress. Different reactions to stress are observed, depending on whether it is of a short-term or long-term nature. The short-term is a natural physiological response, while the long-term tends to be detrimental. Under physiological conditions and in a stress-free context, healthy people release between 10 and 20 mg of cortisol daily, with regular peaks. Initially, there's a rise in ACTH levels, which triggers the release of cortisol. However, when someone experiences prolonged, chronic stress, cortisol levels remain elevated due to more adrenal sensitivity. Thus, when faced with uncontrollable and prolonged stress, it can lead to a range of alterations in various aspects of the CNS, contributing to neuropsychiatric and neurodegenerative conditions. In fact, elevated cortisol secretion during stressful situations can significantly impact the functioning of the brain. This hormone has a pronounced effect on the hippocampus due to the abundance of steroid receptors it possesses. Recurrent exposure to stress triggers alterations in neuronal structures. When stress is acute, the hippocampal atrophy that may occur is often reversible. However, chronic stress can result in neuronal death within the hippocampus. Numerous studies suggested that conditions like depression and post-traumatic stress disorder are associated with a reduction in the volume of the hippocampus, as well as that of the PFC and amygdala.

Glucocorticoid receptors (GRs) and mineralocorticoid receptors (MRs) are the intra-cellular nuclear receptors that respond to cortisol. GRs are found throughout the brain and peripheral tissues, while MRs are primarily located in cardiovascular tissues, the liver, kidneys, and certain brain regions like the corticolimbic areas. These receptors can activate or repress gene transcription in the cell nucleus and also mediate non-genomic effects. During the day, nuclear MRs are typically occupied by cortisol, remaining active. However, during the night, when cortisol levels are very low, these MRs become unoccupied. When cortisol levels rise significantly, both nuclear GRs and membrane-associated MRs and GRs become occupied by this hormone, leading to changes in synaptic plasticity at the cellular level.

Hyperactivity of the HPA axis is one of the most studied findings in MDD. This hyperactivity results in hormones. Elevated glucocorticoid levels can disrupt the body's ability to regulate them effectively.

In individuals with MDD, the GR is typically compromised, leading to diminished negative feedback regulation. This dysregulation leads to increased glucocorticoid synthesis. Antidepressant drugs, on the other hand, also work by influencing the functioning of this system. These influences can take different forms, including regulation of GR expression and post-translational modifications. As a result, the therapeutic effects of antidepressants involve, at least in part, the restoration of the function of GR.

High stress levels have also been associated with disturbances in 5-HT pathways, modifications in the structure of brain regions like the hippocampus and PFC, and epigenetic alterations in genes such as BDNF. Long-term exposure to corticosterone has been demonstrated to induce modifications in the structure of neuronal dendrites, promoting atrophy. Moreover, research on postmortem tissue from individuals with depression and animal models has provided insights into the modifications at the cellular level connected with this condition, particularly structural changes in the brain, including dendritic atrophy, neuronal loss, and alterations in glial elements.

The connection between oxidative stress and MDD's associated stress response is also important in the context of MDD. Increased ROS production leads to hyperactivation of the HPA axis. Furthermore, the release of glucocorticoids caused by HPA axis activation increases the activity of cellular reduction-oxidation systems. When stress activates GRs, there is an increase in mitochondrial membrane potential and mitochondrial oxidation. Consequently, it leads to the generation of ROS, leading to oxidative damage.

Both glucocorticoids and inflammation have been linked to the pathogenesis of depression. According to the glucocorticoid resistance paradigm, less sensitivity to cortisol's anti-inflammatory properties causes increased inflammation in depression. Animal research has indicated that repeated social defeat can produce glucocorticoid-resistant monocytes, depressive-like behaviors in animal models, and enhance neuroinflammatory signaling. Human studies have concurrently observed reduced GR function/expression, HPA axis hyperactivity, and increased inflammation in depressed patients. In fact, depressed people frequently show HPA axis hyperactivity and GR dysfunction, despite elevated inflammatory markers and the glucocorticoids' anti-inflammatory properties. This seeming paradox might be attributed to the fact that the glucocorticoid and inflammatory systems naturally coexist in equilibrium. Chronic stress may alter this balance, favoring inflammatory processes and weakening glucocorticoid signaling. Persistent stress may disrupt the homeostatic balance, causing inflammation while inhibiting the anti-inflammatory effects of glucocorticoids. Also, inflammation and glucocorticoid signaling have separate effects on the same biological processes and structures, resulting in cumulative damage that contributes to depression. In this case, these two systems may not interact directly, but they do converge on shared pathways that lead to depression. Understanding the connection is critical for finding therapy targets in depression.

Additionally, inflammatory changes within the brain have been linked to conditions such as dementia and MDD, highlighting the implications of these interactions. Stress-induced proinflammatory cytokine production also stimulates the indoleamine 2,3-dioxygenase (IDO)/kynurenine (KYN) pathway in cells, including macrophages. IDO plays an important role in the catabolism of TRP, leading to lower 5-HT levels. Particularly noteworthy is the link between elevated concentrations of interleukin (IL)-1 and IL-6 and the stress response. Indeed, an elevated production of IL-6 has been observed to play a role in MDD prognosis and how individuals respond to treatment, possibly through activation of the HPA axis.

The study of the stress response, which is predominantly regulated by the HPA axis, provides a road to understanding the underlying biological mechanisms that are present in depression, allowing for the development of more effective therapies.

The study of the stress response, which is predominantly regulated by the HPA axis, provides a road to understanding the underlying biological mechanisms that are present in depression, allowing for the development of more effective therapies.

Exemplar Nutritional Modulators that Target HPA Stress Response Modulators

Ashwagandha (Withania somnifera): This adaptogen has been extensively studied and shown to reduce cortisol levels, a primary stress hormone, thereby modulating the HPA axis. Clinical trials have demonstrated that doses as low as 125 mg/day can effectively reduce stress in chronically stressed adults.

Saffron (Crocus sativus): Saffron extract has been shown to normalize HPA axis dysregulation and enhance stress resilience in animal models. It reduces adrenocorticotropic hormone and corticosterone levels, indicating its potential in stress mitigation.

Holy Basil (Ocimum tenuiflorum): This herb has demonstrated anti-stress effects by inhibiting cortisol release and antagonizing CRF1 receptors, which are involved in the HPA axis. It has been shown to reduce stress-induced corticosterone levels in animal studies.

Vitamins: such as vitamin C and vitamin E have strong antioxidant properties that can help modulate the HPA axis by combating oxidative stress. These vitamins can help maintain HPA axis homeostasis and reduce cortisol levels. Vitamin D3 has shown some potential in modulating the HPA axis, although the evidence is mixed. A study involving patients with multiple sclerosis found that vitamin D supplementation (4,000 IU/day) led to a reduction in the cortisol awakening response (CAR), suggesting a possible downregulation of HPA axis activity. Another study indicated that vitamin D supplementation during winter helped maintain normal psychophysiological stress responses, although it did not significantly affect cortisol levels. Additionally, animal studies have shown that vitamin D can reduce corticosterone levels and modulate pro-inflammatory cytokines and oxidative stress markers, which are linked to HPA axis activity.

Minerals: Zinc also appears to modulate the HPA axis. Zinc deficiency has been associated with increased glucocorticoid secretion and enhanced HPA axis activity, leading to behavioral abnormalities. Supplementation with zinc has been shown to prevent stress-induced sickness behavior and normalize corticosterone levels in animal models. Furthermore, prenatal zinc supplementation has been found to reduce stress responses in adult offspring exposed to prenatal stress, indicating its potential long-term benefits in HPA axis regulation.

Minerals: Magnesium is crucial for stress management. Supplementation has been shown to improve perceived stress and maintain magnesium homeostasis, which is essential for proper HPA axis function.

Botanicals and Herbs: Botanicals like Rhodiola rosea, Korean ginseng, and Bacopa monnieri have also been studied for their potential to modulate the HPA axis, although the evidence is less consistent compared to Ashwagandha. L-theanine, which modulates the HPA axis by lowering serum cortisol and glutamate levels to reduce chronic stress and its impact on neurons, has shown evidence of effectiveness.

Four exemplary nutritional ingredients and MOA(s) within process pathways for the composition

L-methylfolate supplementation, a key regulator in the one-carbon and methylation cycles, helps to modulate neurotransmitters and receptor synthesis that are affected by chronic activation of the HPA stress response;

Zinc Supplementation can prevent stress-induced sickness behavior and normalize corticosterone levels in animal models;

Vitamin D3 mitigates HPA axis-induced inflammation by regulating the immune system and pro-inflammatory cytokines;

L-theanine modulates the HPA axis, lowering serum cortisol and glutamate levels to reduce chronic stress and its impact on neurons.

### Dosage Forms

Dosage forms and kits according to embodiments herein, target one or more of these six process pathways leading to a mood disorder; 1) neurotransmitter (monoamine), 2) glutamate-GABA, 3) oxidative stress, 4) neuro-inflammation, 5) neurotrophic/synaptic plasticity, 6) HPA stress response, to help bring the processes back into allostasis. Dosage forms, medical foods, dietary supplements and/or methods according to embodiments herein, target one or more of these process pathway systems to help support the body in reaching allostasis and correct nutrient deficits.

According to certain embodiments, the dosage form can be formulated for any suitable route of administration known to those of ordinary skill in the art. In embodiments, the dosage form can be formulated for a route of administration selected from oral, parenteral, subcutaneous, intravenous, intramuscular, intraperitoneal, transdermal, transmucosal (e.g., via the transurethral or rectal routes), sublingual, buccal, gingival, rectal, subcutaneous, transpulmonary, topical, nasal, inhalation and combinations thereof.

According to certain embodiments, dosage forms as disclosed herein may be in the form of a tablet, troche, lozenge, a plurality of units (as described in more detail below), hard capsule, soft capsule, buccal tablet, buccal film, sublingual tablet, gummy chewable, soft chewable, oral powder, sublingual film, liquid (e.g., a liquid shot), etc. In certain embodiments, the dosage form is a shot having a volume of about 1 ml to about 100 ml, about 2 ml to about 75 ml, about 5 ml to about 50 ml, or about 10 ml to about 25 ml. In certain embodiments, the dosage form is a bulk powder to be administered dry or admixed with a liquid, having a dry weight of about 1 gram to about 1000 grams, formulated for reconstitution in liquid, preferably water, in a volume of 1 oz to 16 oz.

According to certain embodiments, dosage forms as disclosed herein can be formulated to have quick or rapidly dissolving properties that would provide an oral sensation of dissolving quickly within the oral cavity. In embodiments, the dosage forms can be formulated to provide rapid delivery (e.g., immediate release) via sub-lingual application incorporating the various components as described herein. In embodiments, oral dosage forms can employ ingredients that produce an effervescent reaction upon placement into liquid, such as water or juice. In yet further embodiments, oral dosage forms described herein can be formulated into a gelatin gummy form. Such “gummies” can incorporate various components that invoke one or more of the following sensations: a cooling or heating sensation within the oral cavity, and bitter, sour and/or sweet taste upon ingestion via oral cavity. In yet further embodiments, dosage forms can be formulated for delivery via a “tea-bag” containing ingredients, as set forth herein. This “tea-bag” form can also employ aromatic oils which are natural oils such as; plant terpenes to stimulate the olfactory system, thus providing a sensory disruption to the sympathetic nervous system.

In certain embodiments, the dosage form can comprise a plurality of units. The plurality of units can include, but are not limited to, particles, powder, granules, beads, microspheres, micro-tablets, microcapsules, extrudates and combinations thereof. In certain embodiments, the plurality of units are in the form of micro-tablets. The micro-tablets can be formed from compressed powder, beds, microspheres and/or granules or can be extrudates. Each of the plurality of units can have a mean size of about 0.1 μm to about 5 mm, or about 1 μm to about 3 mm, or about 50 μm to about 1 mm. In embodiments, the micro-tablets can have a mean size of about 0.5 mm to about 5 mm. The micro-tablets can have any suitable shape known to those of ordinary skill in the art, for example, the micro-tablets can be cylindrical, circular, pyramidal, toroidal and combinations thereof.

According to certain embodiments, the plurality of units can be formulated to provide a controlled release of one or more components therein. The terms “controlled-release,” “extended-release” or “sustained release” are interchangeable and are defined for purposes of the present disclosure as the release of one or more component at such a rate that blood (e.g., plasma) concentrations are maintained within the therapeutic range, but below toxic concentrations, over a period of time of at least about 1 hour or longer, at least about 2 hours or longer, at least about 3 hours or longer, at least about 4 hours or longer, at least about 5 hours or longer, or at least about 6 hours or longer, or at least about 10 hours or longer, or at least about 12 hours or longer or at least about 24 hours or longer. Preferably, a controlled-release dosage form can provide one, two, three, or four times daily dosing.

In certain embodiments, the plurality of units can be formulated to provide an immediate release of one or more components therein. The term “immediate release” is defined for the purposes of the present disclosure as the release of one or more component, “a symptom reliever” selected from one or more “six groups of modulators” (e.g., one or more ingredients known to help relieve symptoms of a mood disorder for example, L-theanine, ketamine) into the air, mouth or in the gastrointestinal contents with no delay or prolongation of dissolution or absorption, smell or taste of the one or more component, intended for rapid relief of symptoms.

In certain embodiments, the dosage form may comprise a pharmaceutically acceptable capsule. In embodiments, at least one of a neurotransmitter modulator, glutamate modulator or HPA stress modulator, can be within the pharmaceutically acceptable capsule. In embodiments where the dosage form comprises a plurality of units, the plurality of units can be contained within the pharmaceutically acceptable capsule. The capsule may be any suitable capsule including a hard shell capsule, a soft shell or a soft gelatin capsule. The pharmaceutically acceptable capsule can be formed of a mixture of natural oils and components.

According to embodiments, the capsule shell can be clear or translucent, or single or multi-colored.

According to certain embodiments, the dosage form provides an immediate release of at least one of the neurotransmitter modulator, glutamate modulator, HPA stress modulator, inflammation modulator, optionally from the oxidative stress, and or neurotrophic synaptic plasticity modulator, and any “modulator” combinations thereof. In embodiments, the dosage form provides a controlled release of at least one of the nutrients intended to return persons deficiency of that nutrient to homeostatic levels, known to occur and contribute to a mood disorder within any of the “modulators” or combinations of the six groups of “modulators”. In further embodiments, the dosage form provides an immediate release of at least one of the deficient nutrients, for example, a vitamin, mineral, enzyme, amino acid, and any combination thereof.

As disclosed above, the dosage form can be formulated for any suitable route of administration known to those of ordinary skill in the art. In embodiments, the dosage form can be in the form of a plurality of units, as discussed above, contained within a soft shell capsule, tablet, caplet or other component. The dosage form may include a controlled release component, an immediate release component or a combination of controlled release and immediate release components as disclosed above.

Dosage forms according to certain embodiments herein can include at least one additional active ingredient as part or a kit (e.g., a drug). Suitable additional active ingredients include, but are not limited to an anti-anxiety drug, an antidepressant, antipsychotic, a prodrug thereof, a cannabinoid, dronabinol, a pharmaceutically acceptable salt thereof, and combinations thereof. Suitable anti-anxiety drugs include, but are not limited to a central nervous system depressant, a benzodiazepine, buspirone, buproprion, a prodrug thereof, a pharmaceutically acceptable salt thereof and combinations thereof. Suitable benzodiazepines include, but are not limited to alprazolam, chlordiazepoxide, clonazepam, diazepam, lorazepam, a prodrug thereof, a pharmaceutically acceptable salt thereof and combinations thereof.

Suitable central nervous system depressants include, but are not limited to a barbiturate, a prodrug thereof, a pharmaceutically acceptable salt thereof and combinations thereof. According to embodiments, the barbiturate includes at least one of secobarbital, butabarbital, mephobarbital, pentobarbital, phenobarbital, amobarbital, a prodrug thereof, a pharmaceutically acceptable salt thereof and combinations thereof. Suitable antidepressants include, but are not limited to; a selective serotonin reuptake inhibitor (SSRI), a selective noradrenaline reuptake inhibitor (SNRI), a tricyclic, a monoamine oxidase inhibitor (MAOI), a beta-blocker, a sedative, a prodrug thereof, a pharmaceutically acceptable salt thereof and combinations thereof. According to embodiments, the SSRI can include at least one of citalopram, escitalopram, fluvoxamine, cerixamine, fenfluramine, paroxetine, dapoxetine, fluoxetine, ifoxetine, vortioxetine, sertraline, vilazodone, cyanodothiepin, nefazodone, a prodrug thereof, a pharmaceutically acceptable salt thereof, and combinations thereof.

Suitable SNRIs include, but are not limited to; venlafaxine, desvenlafaxine, milnacipran, duloxetine, levomilnacipran, reboxetine, desipramine, maprotiline, lofepramine, mirtazapine, oxaprotilin, fezolamine, atomoxetine, bupropion, mianserin, duloxetine, a prodrug thereof, a pharmaceutically acceptable salt thereof and combinations thereof. Suitable tricyclics include but are not limited to; clomiprmamine, imipramine, a prodrug thereof, a pharmaceutically acceptable salt thereof, and combinations thereof. Suitable MAOIs include, but are not limited to; socarboxazid, phenelzine, selegiline, tranylcypromine, a prodrug thereof, a pharmaceutically acceptable salt thereof, and combinations thereof. According to embodiments, the beta-blocker can include at least one of; propranolol, atenolol, a prodrug thereof, a pharmaceutically acceptable salt thereof, and combinations thereof.

In yet further embodiments, disclosed herein are dosage forms, in any known therapeutic dose ranges; comprising the following ingredients: vitamin D3, 1-methylfolate, zinc bisgylcinate, vitamin D3 and 1-theanine. According to embodiments, the dosage form is a pharmaceutically acceptable capsule comprising a combination of natural oils and components as described herein. The ingredients can be in the form of a plurality of units, for example, the ingredients may be prepared as a granulation or powder and formed into compressed micro-tablets or extrudates and other formed types.

### Exemplary Methods of Preparation

Dosage forms according to certain embodiments disclosed herein can be prepared using methods that include, but are not limited to; wet granulation, dry granulation, compression, extrusion, encapsulation, and any other suitable methods known to those of ordinary skill in the art to prepare dosage forms as described herein (i.e., including gummies, topical formulations, liquids, etc.). According to certain embodiments, one or more ingredients of the dosage forms can be prepared in a matrix in the form of particles; a powder, granules, beads, microspheres, and combinations thereof. The granules of each component can be combined into a matrix. Additional ingredients can be added to the matrix as desired. In embodiments, the matrix can be compressed and shaped to form a tablet or a micro-tablet. In further embodiments, the matrix can be converted into an extrudable form and then extruded to form extrudates.

Granules may be formed by wet granulation with water or dry granulation, in an embodiment of a wet granulation process, at least one ingredient selected to target one or more of the six pathways, and are weighed, sifted and mixed (excluding a lubricant), optionally with one or more excipient such as a bulking agent, filler, diluent and disintegrant, in a powder mixer, The ingredients can be mixed using a planetary bowl mixer, ribbon/trough mixer, rotating drum mixer or high-speed mixer until a uniform powder mix (i.e., a matrix) is achieved. The mixing efficiency can be enhanced by the use of powders that have similar average particle size, although this is often not the case in many mixing operations.

Suitable diluents include, but are not limited to; lactose, microcrystalline cellulose, starch, powdered sucrose, mannitol, fructose, sorbitol, calcium phosphate and calcium sulphate. Diluents are usually selected based on the manufacturer's experience with the material, its relative cost, and its compatibility with the drug and other excipients. Suitable disintegrants include, but are not limited to; croscarmellose, sodium starch glycolate, sodium carboxymethylcellulose, polyvinylpyrrolidone (PVP), crospovidone, cation exchange resins, corn and potato starches, alginic acid, and other materials that counteract the effect of binders and the physical forces of compression used in forming the tablets.

The wet granulation method can further include preparing a damp mass. In embodiments, the amount of water added during the wet granulation can be about 1.5 to about 5 times, or about 1.75 to about 3.5 times the dry weight of the matrix. The binder solution can be mixed with the powder mixture to form an adhesive mass, which can be granulated. The amount of binding agent used, as well as the quantity of fluid required to form a damp and coherent mass, is known to those of ordinary skill in the art. The resulting binder-powder mixture should compact when squeezed in the hand. The use of insufficient binder tends to poor adhesion, capping and soft tablets. Excessive binder solution yields hard tablets with slow disintegrating properties. Suitable granulating agents are solutions of; povidone, an aqueous preparation of cornstarch, molasses, methylcellulose, hydroxypropyl methylcellulose, carboxymethylcellulose, glucose solution and microcrystalline cellulose. Suitable dry binders or nonaqueous solution can be used for substances that are adversely affected by aqueous solution. Colorants or flavoring agents can be added to the binding agent to prepare a granulation with an added feature.

The dampened powder can be wet screened into pellets or granules. The wet-massed powder blend can be screened on a 6- to 12-mesh screen to prepare wet granules. This may be done by hand or with suitable equipment that prepares the granules by extrusion through perforations in the apparatus. The granules formed are spread evenly on trays and dried in an oven.

The screened moist granules are then dried in an oven at a controlled temperature not exceeding 55° C. to a consistent weight or constant moisture content. The drying temperature and the duration of drying process depend on the nature of the active ingredient and the level of moisture required for the successful production of satisfactory tablets. Shelf or tray drier and fluidized-bed drier can be used for this purpose.

In certain embodiments, the dried granules are passed through a screen of smaller size than that used to prepare the moist granules. The size of the final granules is dependent on the size of the punches (and hence the final tablet size). Screens of 14- to 20-mesh size are generally used for this purpose. After dry screening, the dried and screened granules can be separated into coarse and fine granules by shaking them on a 250-mesh sieve. An appropriate quantity of lubricant is passed through a 200-mesh sieve. This is mixed with the fine granules before the coarse granules are incorporated. The quantity of lubricant used varies from one formulation scientist to another but usually ranges from about 0.1% to 5% of the weight of the granulation. Suitable lubricants for use in wet granulation include, but are not limited to; magnesium stearate, calcium stearate, stearic acid, wax, hydrogenated vegetable oil, talc, and starch.

A suitable dry granulation process includes weighing formulation ingredients in appropriate quantities. The excipients and active ingredient(s) should be in finely divided form, otherwise, particle size reduction should be carried out.

The weighed formulation ingredients are mixed in a powder mixer until a uniform powder mix is achieved. In embodiments, half the lubricant in the formula can be added at this stage to enhance powder flow during slugging and to prevent the sticking of compressed powder on the die during precompression.

The mixed ingredients can then be compressed into flat large tablets or pellets, referred to as precompression (a.k.a., slugging) and the compacts made in the process typically have sizes of about 25 mm diameter by about 10-15 mm thick. Compression of mixed powders into slugs can be achieved by slugging technique or roller compaction. The pressure used to produce the slugs is usually less than that used in the final compression.

Following slugging, the slugs can be broken into smaller pieces using a hammer mill or other conventional milling equipment. The milled slugs are screened to produce uniform granules. After screening, the remaining lubricant and other extragranular excipients such as disintegrant, glidant etc., as described herein, can be added to the granulation and mixed gently to achieve a uniform blend. The mixed granules can be compressed into tablets using either a single or rotary tablet press fitted with appropriate punches and dies. Similar to wet granulation, tablets manufactured by dry granulation method may be coated if the need arises. The disintegrant can be added to the weighed formulation at the beginning of the process (intragraular) or to the screened milled slugs (extragranular) and sometimes in both steps (intragranular extragranular). The formed granules can then be compressed into tablets or micro-tablets.

In certain embodiments, matrices can be formed using a melt-granulation or melt-extrusion technique. Generally, melt-granulation techniques involve melting a normally solid binder material, e.g. a wax, and incorporating a powder therein. Other ingredients can be added, for example, release modifying agents, diluents, lubricants, binders, granulating aids, colorants, flavorants and glidants that are conventional in the pharmaceutical art in amounts up to about 50% by weight of the particulate if desired. The quantities of these additional materials will be sufficient to provide the desired effect in the desired formulation.

According to certain embodiments, methods of preparing dosage forms as disclosed herein can include a number of pharmaceutically acceptable preparation techniques (e.g., granulation, extrusion, compression, coating, encapsulation, etc. as discussed above). For example, the matrix can be compressed into tablets in a single rotary tablet press with a plurality of punch pairs. In embodiments, the micro-tablets may then be encapsulated within a pharmaceutically acceptable capsule (e.g., a soft gelatin capsule).

In certain embodiments, the kit could be comprised of a “modulation therapy” device, for example, a Transmagnetic stimulation device, digital devices for stimulation of the autonomic nervous system, massage devices, and other external device or treatment that externally applied.

### Methods of Use

Further disclosed herein are methods of using dosage forms and/or kits according to embodiments herein. In certain embodiments, disclosed are methods of supporting a human body's response and nutritional status to any depressive type disorder, including bipolar disorder, that include administering to a human a dosage form, which may be a part of a kit, according to embodiments herein. In embodiments, the dosage form can be administered by the human as needed when the human is suffering from a depressive disorder. In certain embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any anti-psychotic type disorder, that include administering to a human a dosage form, which may be a part of a kit, according to embodiments herein. In embodiments, the dosage form can be administered by the human as needed when the human is suffering from depressive disorder. In certain embodiments, the dosage form can be administered by the human in the morning, afternoon, or evening. According to certain embodiments, the human can be administered any safe and suitable amount. In certain embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any anxiety or stress type disorder, that include administering to a human a dosage form, which may be a part of a kit, according to embodiments herein. In embodiments, the dosage form can be administered by the human as needed when the human is suffering from depressive disorder. In embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any obsessive compulsive type disorder, that include administering to a human a dosage form, which may be a part of a kit, according to embodiments herein. In embodiments, the dosage form can be administered by the human as needed when the human is suffering from depressive disorder. In embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any attention type disorder, that include administering to a human a dosage form, which may be a part of a kit, according to embodiments herein. In embodiments, the dosage form can be administered by the human as needed when the human is suffering from depressive disorder. In embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any autism spectrum type disorder, that include administering to a human a dosage form, which may be a part of a kit, according to embodiments herein. In embodiments, the dosage form can be administered by the human as needed when the human is suffering from depressive disorder. In embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Further disclosed herein are methods of using dosage forms and/or kits according to embodiments herein. In embodiments, disclosed are methods of supporting a human body's response and nutritional status to any obsessive compulsive disorder, that include administering to a human a dosage form, which may be a part of a kit, according to embodiments herein. In embodiments, the dosage form can be administered by the human as needed when the human is suffering from depressive disorder. In embodiments, the dosage form can be administered by the human in the morning, afternoon, or evening. According to various embodiments, the human can be administered any safe and suitable amount. In embodiments, the human can be administered up to 1 serving, up to 2 servings, up to 3 servings, or up to 4 servings, daily of the dosage form where each serving is, for example, 1 or 2 capsules, 1 or 2 tablets, 1 or 2 shots, etc.

Additional methods of treatment and benefits of the dosage forms and kits disclosed herein include the clinical dietary management of mood disorders; providing the nutritional requirements associated with neurotransmitter imbalances in mood disorders; the use alongside SSRIs/SNRIs to help restore biochemical balance supportive of treatment response.

Subjects with specific risk factors that may benefit from the dosage forms and kits of the present invention include subjects with partial SSRI/SNRI response, BMI ≥30, elevated inflammatory markers, relevant genetic variants (e.g., MTHFR), limited sunlight exposure, alcohol/tobacco use, high stress, or taking medications linked to nutrient depletion (e.g., metformin, oral contraceptives, anticonvulsants).

In certain embodiments, the dosage forms and kits of the present invention are appropriate for individuals who prefer nutritional or integrative approaches to mood management. In other embodiments, the dosage forms and kits of the present invention are administered to adults and adolescents ages 12 and older, e.g., once daily (OD), twice daily (BID), three times daily (TID) or for times daily (QID).

In certain embodiments, the dosage forms and kits of the present invention provide support to one or more of mood regulation, neuroprotection, cognitive function, stress management, and mental resilience (neuroplasticity) by addressing nutrient deficiencies that can disrupt neurotransmission.

In certain embodiments, the dosage forms and kits of the present invention support neurotransmitter homeostasis and aim to balance glutamate, GABA, serotonin, dopamine, and norepinephrine through targeted nutritional pathways.

In certain embodiments, the dosage forms and kits of the present invention support monoamine synthesis (serotonin, dopamine, norepinephrine) via methylation/SAMe pathways; reduces homocysteine and oxidative stress; and modulates glutamate/NMDA activity.

In certain embodiments, the dosage forms and kits of the present invention help to regulate the HPA axis, modulate glutamate signaling/NMDA function, support neuroplasticity, and aid serotonin/dopamine synthesis.

In certain embodiments, the dosage forms and kits of the present invention modulate serotonin synthesis and immune signaling.

In certain embodiments, the dosage forms and kits of the present invention support stress reduction and relaxation (reduced cortisol, increased alpha brain-wave power); improves sleep quality and aspects of cognition (executive function, verbal fluency); and supports BDNF and neuroplasticity.

In certain embodiments, the dosage forms and kits of the present invention are one or more of dye-free, sugar-free, non-GMO, vegetarian, and free of major allergens.

In certain embodiments, the dosage forms and kits of the present invention may be utilized for neurotransmitter-imbalance mood disorders such as major depressive disorder (including SSRI-inadequate responders), treatment-resistant depression, and schizophrenia (including negative symptoms).

In certain embodiments, the dosage forms and kits of the present invention may be utilized for anxiety and stress-related conditions such as general anxiety symptoms, stress reduction, and sleep disturbances associated with mood and anxiety disorders.

In certain embodiments, the dosage forms and kits of the present invention may be utilized for cerebral folate deficiency (CFD) and related neurologic/psychiatric conditions such as neurologic and psychiatric diseases associated with low CSF 5-MTHF (e.g., intractable childhood epilepsy, movement disorders, psychomotor retardation, intellectual disability, autism spectrum disorders, Rett syndrome, Aicardi-Goutières syndrome, cerebral hypomyelination, and mitochondrial disorders.

In certain embodiments, the dosage forms and kits of the present invention may be utilized for epileptic syndromes and seizures associated with CFD.

In certain embodiments, the dosage forms and kits of the present invention may be utilized for cognitive and neurodegenerative contexts linked to vitamin D deficiency, depression with cognitive impairment; dementia, Alzheimer's disease, Parkinson's disease, and multiple sclerosis.

In certain embodiments, the dosage forms and kits of the present invention may be utilized for perinatal depression.

In certain embodiments, the dosage forms and kits of the present invention may be utilized for obsessive-compulsive disorder.

In certain embodiments, the dosage forms and kits of the present invention may be utilized for attention deficit disorder (ADD) or attention deficit hyperactivity disorder (ADHD).

In certain embodiments, the dosage forms and kits of the present invention may be utilized for tardive dyskinesia.

### Summary of Exemplary Ingredients and Range of Doses for a Multi-Action Formula

### I-Methylfolate (e.g., 1 mg to 75 mg; or 15 mg)

(a) The role of folate in the body
   I. Folates, in the form of tetrahydrofolates (such as L-methylfolate), serve as enzyme cofactors that carry one-carbon units. One-carbon metabolism is necessary for the de novo synthesis of purines, numerous methylation reactions, including DNA methylation and neurotransmitter synthesis and degradation, and for DNA synthesis and genome stability. These are critical biochemical functions that are the basis of cell activity that when imbalanced in the body may result in manifestation of disease.
(b) the Impact of Folate Deficiency on the Brain
   I. Folate is essential for brain health. Brain folate deficiency can occur in the absence of whole-body folate deficiency.
   II. The brain is susceptible to folate deficiency due to impairments in both folate metabolism and/or energy metabolism. This is because the body concentrates folate across the blood-brain barrier by up to 3-fold (serum/cerebrospinal fluid), which is an energy requiring process. Patients with inborn errors of metabolism affecting folate utilization, energy metabolism, or folate transport across the blood-brain are at increased risk for cerebral folate deficiency and subsequent neurological and neuropsychiatric conditions even in the absence of whole-body deficiency. Similarly, decreased folate transport activity due to autoimmunity or compromised blood-brain barrier function can result in similar biochemical and clinical sequelae.
(c) Use of High-Dose Folate in Folate Deficiency
   I. Administration of high-dose reduced folate, which includes L-methylfolate, to patients with folate receptor autoimmunity as well as populations with folate-related genetic mutations for MTHFR gene, restores cerebrospinal fluid folate levels and is associated with improvements in clinical conditions as evidenced by seizure control and normalization of brain metabolites as shown by MRS, white matter lesions and.
   II. High-dose folic acid is not interchangeable with reduced folate, like L-methylfolate. High-dose folic acid is not effective and not recommended in cases of cerebral folate deficiency due to low rates of folic acid transport across the blood-brain barrier and low brain levels of dihydrofolate reductase, the enzyme required to convert folic acid into a biological and functional cofactor. Furthermore, folic acid in physiological doses can inhibit the transport of L-methylfolate across the blood-brain barrier. Finally, high-dose folic acid may cause nephrotoxicity.
   III. Patients with depression or schizophrenia are more likely to have common genetic variants that contribute to folate deficiency, and many of these polymorphisms are also associated with neuropsychiatric disease. For example, the 677C>T methylenetetrahydrofolate reductase (MTHFR) polymorphism impacts both the functioning of folate-mediated one-carbon metabolism as well as folate status which has been associated with an increased risk of depression (odds ratio=1.36, 95% CI 1.11-1.67) and schizophrenia (odds ratio=1.44, 95% CI 1.21-1.70); up to 70% percent of patients with either depression or schizophrenia have been shown to have some form of the polymorphism. Other polymorphisms that have been associated with either depression or schizophrenia include, catechol-O-methyltransferase, folate hydrolase 1, and methionine synthase. Patients with depression or schizophrenia are more likely to have autoimmunity to folate receptors. The odds of having autoantibodies to folate receptor α, the folate transporter responsible for folate uptake in the brain, in 101 patients with depression not responding to first line antidepressant treatment were significantly greater compared age and sex match healthy controls (odds ratio=3.24, 95% CI 1.70-6.33). In another study, 18 patients diagnosed with schizophrenia, 15 (83.3%) tested positive for serum FRa autoantibodies compared to only 1 in 30 healthy controls (3.3%) (χ2=21.6; p<0.0001).
   IV. The clinical benefits of L-methylfolate administration under medical supervision via prescription for patients with Patients With A Neurotransmitter Imbalance Leading To A Mood Disorder such as; depression or schizophrenia, to address associated imbalances in folate metabolism.
(d) L-methylfolate circumvents the absorption, transport and accretion issues resulting from the genetic polymorphisms and autoimmunity seen in patients with depression or schizophrenia. Clinical studies have shown that nutritional intervention with high dose oral L-methylfolate resulted in improved neuropsychiatric symptoms observed in patients with depression or schizophrenia as assessed through well-recognized clinical evaluation scales. Since most studies in these populations were conducted in populations not stratified by genetic polymorphism or the presence of absorption-blocking autoantibodies (both factors that affect L-methylfolate uptake/metabolism in the brain), the effect in these individuals may be even more pronounced. ZINC (1 mg to 100 mg; preferably 25 mg) The role of Zinc in the body (Zinc (e.g., 1 mg to 100 mg; or 25 mg)

Zinc is involved in many aspects of cellular metabolism. It is required for the catalytic activity of hundreds of enzymes, and it plays a role in enhancing immune function, protein and DNA synthesis, wound healing, and cell signaling and division. Zinc also supports healthy growth and development during pregnancy, infancy, childhood, and adolescence, and is involved in the sense of taste. Because zinc has many functions throughout the body, zinc deficiency affects many different tissues and organs. Zinc deficiency can affect, for example, skin; bones; and the digestive, reproductive, central nervous, and immune systems.
I. Zinc and iron are important in regulating cellular function and neuromodulation. Zinc may also have an influence on the neural transmission involved in depression, such as the serotonergic, dopaminergic and glutamatergic systems.
II. The antidepressant properties of zinc may be explained by modulating the functions of serotonergic and N-methyl-D-aspartate (NMDA) receptors and increasing levels of brain derived neurotrophic factor (BDNF).
(b) The impact of Zinc deficiency on the brain
I. There may be an inverse association between dietary zinc intake and risk of depression.
II. Depleted zinc levels enhance HPA activity, which causes a surge in glucocorticoids, ultimately inducing hippocampal dysfunction and behavioral abnormalities. Hyperactivation of the HPA axis can cause an imbalance in serotonergic and noradrenergic circuits, affecting mood.
III. Zinc deficiency may contribute to developing depressive disorders by increasing cortisol levels, decreasing neurogenesis and neuroplasticity, and disturbing glutamate homeostasis.
IV There may be an inverse association between dietary zinc intake and risk of depression, even after adjusting for potential confounders.
(c) Use of high dose Zinc administration to address associated imbalances in folate metabolism

Supplementation with, e.g., 25 to 75 mg/day of zinc may be beneficial in depressed patients.

In one instance, 3 months of supplementation with, e.g., 30 mg/day of zinc, and e.g., 2,000 IU vitamin D, either individually or in combination, may help depressed patients to improve mood and depression severity

### Depression

I. Increasing serum zinc levels in depressed individuals may have positive outcomes.
II. An inverse relationship may exist between lower zinc levels and higher Hamilton Depression Rating Scale scores.
III Lower serum zinc levels may be associated with major depression.

### Schizophrenia:

I. Has neurodevelopmental underpinnings and prenatal zinc deficiency may also be relevant, whether a consequence of maternal zinc insufficiency or fetal gene variants that impact the movement of zinc across cellular membranes.
II. Prenatal zinc deficiency produces decreased brain volume in rodent models, consistent with impaired cell proliferation and retarded neuronal maturation. Risk of schizophrenia may be associated with brain zinc content.
III. The expression of the schizophrenia phenotype may reflect interactions of prenatal zinc deficiency with other risk genes, and/or ongoing deficiency following birth.
IV. In addition, intracellular deficiency may arise from low circulating zinc levels due to dietary insufficiency, or impaired absorption from aging or medical conditions, including alcoholism.
V. A host of medications commonly administered to psychiatric patients, including anticonvulsants, oral medications for diabetes, hormones, antacids, anti-inflammatories, and others, also impact zinc absorption.
VI. Furthermore, inefficient genetic variants in zinc transporter molecules that transport the ion across cellular membranes impede its action even when circulating zinc concentrations is in the normal range. Findings from a meta-analysis of studies on zinc deficiency and zinc supplementation in relation to psychosis found that well powered clinical studies, have shown beneficial effects of supplemental zinc in depression and concluded that the meta-analyses support the adjunctive use of zinc in the dietary management major depression and for psychotic symptoms.

### Use of Zinc in Post-Partum Depression

Postpartum zinc supplementation may significantly improve the status of maternal blood zinc levels and reduced the risk of developing postpartum depression.

### Vitamin D (e.g., 1000IU to 100,000IU; or 2000IU)

### (a) the Role of Vitamin D in the Body

Vitamin D has other roles in the body, including reduction of inflammation as well as modulation of such processes as cell growth, neuromuscular and immune function, and glucose metabolism. Many genes encoding proteins that regulate cell proliferation, differentiation, and apoptosis are modulated in part by vitamin D.

### (a) the Impact of Vitamin D Deficiency on the Brain

Vitamin D can have effects on the central nervous system. Vitamin D receptors are widely distributed in areas of the brain, such as the amygdala and substantia nigra. Vitamin D is able to cross the blood-brain barrier, activate vitamin D receptors, and play a role in human behavior control.

Vitamin D acts as a neuro-immunomodulator, influencing neuroinflammation, oxidative stress, and neurotransmitter synthesis. It helps regulate the production of neurotrophic factors, which are essential for neuronal growth, survival, and synaptic plasticity. Deficiency in vitamin D can lead to increased neuroinflammation, oxidative stress, and impaired neurotransmission, contributing to cognitive decline and increased risk of dementia.

### (a) Use of High Dose Vitamin D3 Administration to Address Associated Imbalances in Vitamin D

Vitamin D deficiency has significant implications for brain health, impacting cognitive function and increasing the risk of neurodegenerative diseases. Vitamin D deficiency can have an increased risk of cognitive impairment, dementia, and Alzheimer's disease.

Vitamin D plays a role in neuroinflammation, which is a critical immune response in the brain. Chronic neuroinflammation can contribute to neurodegenerative and psychiatric mood related disorders. Vitamin D modulates neuroinflammation by influencing the production of proinflammatory cytokines and antioxidants, potentially mitigating neuronal damage. While vitamin D is associated with calcium homeostasis and bone health, it also exerts immunomodulatory and neuroprotective effects within the central nervous system. Through comprehensive analysis of preclinical and clinical studies, researchers have reported that vitamin D, acting through its receptors in glial cells, may influence the production of proinflammatory cytokines and antioxidants, potentially mitigating the cascade of events leading to neuronal damage.

Neuroinflammation is a crucial immune response in the brain against injuries or infections, but its persistence can lead to diseases such as Alzheimer's, Parkinson's, multiple sclerosis, and depression. Cholecalciferol (Vitamin D3) is a regulator of neuroinflammation, present in brain cells such as astrocytes and microglia, modulating immune function. Vitamin D's mechanisms of action include cytokine modulation and regulation of nuclear and mitochondrial genes. It adjusts inflammatory mediators and antioxidants, resulting in neuroprotective effects. Additionally, vitamin D impacts neurotransmitter synthesis and brain plasticity. This positions vitamin D as a potential adjunct in treating diseases like Alzheimer's and Parkinson's. Lastly, its role in intestinal microbiota and serotonin synthesis contributes to psychiatric disorders like schizophrenia and depression. Thus, vitamin D presents a positive therapeutic approach for neuroinflammatory, neurodegenerative, and neuropsychiatric diseases.

Vitamin D deficiency is a common thread in the increased risks of multiple sclerosis, Parkinson's disease, Alzheimer's, and depression, among other mood related disorders. Furthermore, preclinical models suggest vitamin D's regulatory capacity over inflammatory mediators, its protective role against neuronal apoptosis, and its contribution to neurogenesis and synaptic plasticity. These insights underscore the potential of vitamin D supplementation not only in slowing the progression of neurodegenerative diseases but also in improving the quality of life for patients suffering from psychiatric conditions.

Low vitamin D status is associated with adverse neuroimaging outcomes, such as reduced gray and white matter volumes, and an increased risk of dementia. There may be a causal relationship between vitamin D deficiency and dementia, emphasizing the importance of maintaining adequate vitamin D levels for brain health.

Vitamin D deficiency negatively impacts cognitive function and increases the risk of neurodegenerative diseases through mechanisms involving neuroinflammation and brain structural changes. Maintaining optimal vitamin D levels is crucial for preserving cognitive health and preventing neurodegenerative conditions.

### L-THEANINE (e.g., 25 mg to 500 mg; or 250 mg)

L-theanine (γ-glutamylethylamide) was discovered as a constituent of green tea in 1949 by Sakato and in 1964 was approved as a food additive in Japan. Because of its various health functions, 1-theanine has been commercially developed as a valuable ingredient easily used for various applications in food and pharmaceutical industries. L-theanine may have antipsychotic-like and possibly antidepressant-like effects.

Regarding its clinical features, inhibition of the central nervous system mediated by the inhibitory neurotransmitter gamma-aminobutyric acid (GABA) may be associated with the anxiolytic effects of L-theanine, and such association may be helpful for sleep disturbances, particularly in MDD individuals. Hidese and colleagues found that 1-theanine administration reduced depressive symptoms and improved cognitive function in MDD patients.

### (a) L-Theanine's Multiple Mechanisms of Action: Overview

It is a water-soluble compound and when ingested orally is absorbed in the small intestine. In rats, peak plasma concentration was found 30 minutes after oral dosing. Theanine crosses the blood-brain barrier via the large neutral amino acid (leucine-preferring) transport system.

L-theanine may have antipsychotic-like and possibly antidepressant-like effects. At molecular level, 1-theanine appears to stimulate brain-derived neurotrophic factor (BDNF) in the hippocampus whilst agonistically acting on NMDA receptors, having also a modulatory effect on central monoaminergic neurotransmitter systems. Regarding its clinical features, inhibition of the central nervous system mediated by the inhibitory neurotransmitter gamma-aminobutyric acid (GABA) may be associated with the anxiolytic effects of 1-theanine, and such association may be helpful for sleep disturbances, particularly in MDD individuals.
1 Neurotransmitter Modulation 1-theanine can influence the levels of key monoamine neurotransmitters such as serotonin (5-HT), norepinephrine (NE), and dopamine (DA). L-theanine administration may increase the levels of these neurotransmitters in brain regions associated with mood regulation, such as the prefrontal cortex, nucleus accumbens, and hippocampus.
2 Brain-Derived Neurotrophic Factor (BDNF) L-theanine can increase the expression of BDNF in the hippocampus, which is beneficial for neuroplasticity and may be useful in the pathophysiology of depression.
3. NMDA Receptor Modulation L-theanine acts as an agonist at NMDA receptors, which are involved in synaptic plasticity and cognitive functions. This action may contribute to its antidepressant-like effects.
4. Anxiolytic Effects L-theanine can reduce anxiety-like behaviors in animal models. L-theanine downregulates the hypothalamic-pituitary-adrenal (HPA) axis involved in the stress response through several mechanisms. It enhances hippocampal activity and alters the cerebrospinal fluid amino acid profile, which may contribute to its anxiolytic effects.
5. Anti-inflammatory activity: L-theanine may inhibit the nuclear factor kappa B (NF-κB) pathway, thereby reducing the expression of proinflammatory factors, including cyclooxygenase-2 (COX-2), prostaglandin E2, inducible nitric oxide synthase, as well as NO, and protect the degradation of extracellular matrix
6. Increases alpha-brain wave activity: L-theanine may increase alpha-brain wave activity, a sign of induced relaxation. In the brain L-theanine increases dopamine and serotonin production.
   (a) Clinical benefits of administering Theanine in patients with neurotransmitter imbalances resulting in a mood disorder

L-theanine may be used as an adjunct to conventional antidepressants. L-theanine may exert its effects on mood disorders through multiple mechanisms, including modulation of neurotransmitter levels, enhancement of BDNF expression, and NMDA receptor activity.

### OTHER EMBODIMENTS

In certain embodiments, the pharmaceutical composition comprises L-methylfolate in an amount from about 1 mg to about 50 mg, about 5 mg to about 25 mg, about 10 mg to about 20 mg or about 15 mg.

In certain embodiments, the pharmaceutical composition disclosed herein maintains at least 90% of the amount of L-methylfolate after storage for 1 month at 40° C.±2° C. and 75%±5% RH.

In certain embodiments, the pharmaceutical composition disclosed herein maintains at least 95% of the amount of L-methylfolate after storage for 1 month at 40° C.±2° C. and 75%±5% RH.

In certain embodiments, the pharmaceutical composition disclosed herein maintains at least 98% of the amount of L-methylfolate after storage for 1 month at 40° C.±2° C. and 75%±5% RH.

In certain embodiments, the pharmaceutical composition disclosed herein maintains 100% of the amount of L-methylfolate after storage for 1 month at 40° C.±2° C. and 75%±5% RH.

In certain embodiments, the pharmaceutical composition disclosed herein maintains at least 90% of the amount of L-methylfolate after storage for 2 months at 40° C.±2° C. and 75%±5% RH.

In certain embodiments, the pharmaceutical composition disclosed herein maintains at least 95% of the amount of L-methylfolate after storage for 2 months at 40° C.±2° C. and 75%±5% RH.

In certain embodiments, the pharmaceutical composition disclosed herein maintains at least 98% of the amount of L-methylfolate after storage for 2 months at 40° C.±2° C. and 75%±5% RH.

In certain embodiments, the pharmaceutical composition disclosed herein maintains 100% of the amount of L-methylfolate after storage for 2 months at 40° C.±2° C. and 75%±5% RH.

In certain embodiments, the pharmaceutical composition disclosed herein maintains at least 90% of the amount of L-methylfolate after storage for 3 months at 40° C.±2° C. and 75%±5% RH.

In certain embodiments, the pharmaceutical composition disclosed herein maintains at least 95% of the amount of L-methylfolate after storage for 3 months at 40° C.±2° C. and 75%±5% RH.

In certain embodiments, the pharmaceutical composition disclosed herein maintains at least 98% of the amount of L-methylfolate after storage for 3 months at 40° C.±2° C. and 75%±5% RH.

In certain embodiments, the pharmaceutical composition disclosed herein maintains 100% of the amount of L-methylfolate after storage for 3 months at 40° C.±2° C. and 75%=5% RH.

In certain embodiments, the pharmaceutical composition disclosed herein maintains at least 90% of the amount of L-methylfolate after storage for 6 months at 40° C.±2° C. and 75%±5% RH.

In certain embodiments, the pharmaceutical composition disclosed herein maintains at least 95% of the amount of L-methylfolate after storage for 6 months at 40° C.±2° C. and 75%±5% RH.

In certain embodiments, the pharmaceutical composition disclosed herein maintains at least 98% of the amount of L-methylfolate after storage for 6 months at 40° C.±2° C. and 75%±5% RH.

In certain embodiments, the pharmaceutical composition disclosed herein maintains 100% of the amount of L-methylfolate after storage for 6 months at 40° C.±2° C. and 75%±5% RH.

In certain embodiments, the pharmaceutical composition disclosed herein provides a dissolution of at least 75% L-methylfolate at 60 minutes under USP <2040> conditions.

In certain embodiments, the pharmaceutical composition disclosed herein provides a dissolution of at least 85% L-methylfolate at 60 minutes under USP <2040> conditions.

In certain embodiments, the pharmaceutical composition disclosed herein provides a dissolution of at least 95% L-methylfolate at 60 minutes under USP <2040> conditions.

In certain embodiments, the pharmaceutical composition disclosed herein provides a dissolution of 100% L-methylfolate at 60 minutes under USP <2040> conditions.

In certain embodiments, the pharmaceutical composition disclosed herein provides a dissolution of at least 75% L-methylfolate at 60 minutes under USP <2040> conditions after storage for 1 month at 40° C.±2° C. and 75%±5% RH

In certain embodiments, the pharmaceutical composition disclosed herein provides a dissolution of at least 85% L-methylfolate at 60 minutes under USP <2040> conditions after storage for 1 month at 40° C.±2° C. and 75%±5% RH.

In certain embodiments, the pharmaceutical composition disclosed herein provides a dissolution of at least 95% L-methylfolate at 60 minutes under USP <2040> conditions after storage for 1 month at 40° C.±2° C. and 75%±5% RH.

In certain embodiments, the pharmaceutical composition disclosed herein provides a dissolution of 100% L-methylfolate at 60 minutes under USP <2040> conditions after storage for 1 month at 40° C.±2° C. and 75%±5% RH.

In certain embodiments, the pharmaceutical composition disclosed herein provides a dissolution of at least 75% L-methylfolate at 60 minutes under USP <2040> conditions after storage for 2 months at 40° C.±2° C. and 75%±5% RH

In certain embodiments, the pharmaceutical composition disclosed herein provides a dissolution of at least 85% L-methylfolate at 60 minutes under USP <2040> conditions after storage for 2 months at 40° C.±2° C. and 75%±5% RH.

In certain embodiments, the pharmaceutical composition disclosed herein provides a dissolution of at least 95% L-methylfolate at 60 minutes under USP <2040> conditions after storage for 2 months at 40° C.±2° C. and 75%±5% RH.

In certain embodiments, the pharmaceutical composition disclosed herein provides a dissolution of 100% L-methylfolate at 60 minutes under USP <2040> conditions after storage for 2 months at 40° C.±2° C. and 75%±5% RH

In certain embodiments, the pharmaceutical composition disclosed herein provides a dissolution of at least 75% L-methylfolate at 60 minutes under USP <2040> conditions after storage for 3 months at 40° C.±2° C. and 75%±5% RH

In certain embodiments, the pharmaceutical composition disclosed herein provides a dissolution of at least 85% L-methylfolate at 60 minutes under USP <2040> conditions after storage for 3 months at 40° C.±2° C. and 75%±5% RH.

In certain embodiments, the pharmaceutical composition disclosed herein provides a dissolution of at least 95% L-methylfolate at 60 minutes under USP <2040> conditions after storage for 3 months at 40° C.±2° C. and 75%±5% RH.

In certain embodiments, the pharmaceutical composition disclosed herein provides a dissolution of 100% L-methylfolate at 60 minutes under USP <2040> conditions after storage for 3 months at 40° C.±2° C. and 75%±5% RH

In certain embodiments, the pharmaceutical composition disclosed herein provides a dissolution of at least 75% L-methylfolate at 60 minutes under USP <2040> conditions after storage for 6 months at 40° C.±2° C. and 75%±5% RH

In certain embodiments, the pharmaceutical composition disclosed herein provides a dissolution of at least 85% L-methylfolate at 60 minutes under USP <2040> conditions after storage for 6 months at 40° C.±2° C. and 75%±5% RH.

In certain embodiments, the pharmaceutical composition disclosed herein provides a dissolution of at least 95% L-methylfolate at 60 minutes under USP <2040> conditions after storage for 6 months at 40° C.±2° C. and 75%±5% RH.

In certain embodiments, the pharmaceutical composition disclosed herein provides a dissolution of 100% L-methylfolate at 60 minutes under USP <2040> conditions after storage for 6 months at 40° C.±2° C. and 75%±5% RH.

In certain embodiments, the pharmaceutical composition disclosed herein comprises L-theanine in an amount from about 50 mg to about 1000 mg, about 100 mg to about 500 mg, about 200 mg to about 300 mg or about 250 mg.

In certain embodiments, the pharmaceutical composition disclosed herein maintains at least 90% of the amount of L-theanine after storage for 1 month at 40° C.±2° C. and 75%±5% RH.

In certain embodiments, the pharmaceutical composition disclosed herein maintains at least 95% of the amount of L-theanine after storage for 1 month at 40° C.±2° C. and 75%±5% RH.

In certain embodiments, the pharmaceutical composition disclosed herein maintains at least 98% of the amount of L-theanine after storage for 1 month at 40° C.±2° C. and 75%±5% RH.

In certain embodiments, the pharmaceutical composition disclosed herein maintains 100% of the amount of L-theanine after storage for 1 month at 40° C.±2° C. and 75%±5% RH.

In certain embodiments, the pharmaceutical composition disclosed herein maintains at least 90% of the amount of L-theanine after storage for 2 months at 40° C.±2° C. and 75%±5% RH.

In certain embodiments, the pharmaceutical composition disclosed herein maintains at least 95% of the amount of L-theanine after storage for 2 months at 40° C.±2° C. and 75%±5% RH.

In certain embodiments, the pharmaceutical composition disclosed herein maintains at least 98% of the amount of L-theanine after storage for 2 months at 40° C.±2° C. and 75%±5% RH.

In certain embodiments, the pharmaceutical composition disclosed herein maintains 100% of the amount of L-theanine after storage for 2 months at 40° C.±2° C. and 75%±5% RH.

In certain embodiments, the pharmaceutical composition disclosed herein maintains at least 90% of the amount of L-theanine after storage for 3 months at 40° C.±2° C. and 75%±5% RH.

In certain embodiments, the pharmaceutical composition disclosed herein maintains at least 95% of the amount of L-theanine after storage for 3 months at 40° C.±2° C. and 75%±5% RH.

In certain embodiments, the pharmaceutical composition disclosed herein maintains at least 98% of the amount of L-theanine after storage for 3 months at 40° C.±2° C. and 75%±5% RH.

In certain embodiments, the pharmaceutical composition disclosed herein maintains 100% of the amount of L-theanine after storage for 3 months at 40° C.±2° C. and 75%±5% RH.

In certain embodiments, the pharmaceutical composition disclosed herein maintains at least 90% of the amount of L-theanine after storage for 6 months at 40° C.±2° C. and 75%±5% RH.

In certain embodiments, the pharmaceutical composition disclosed herein maintains at least 95% of the amount of L-theanine after storage for 6 months at 40° C.±2° C. and 75%±5% RH.

In certain embodiments, the pharmaceutical composition disclosed herein maintains at least 98% of the amount of L-theanine after storage for 6 months at 40° C.±2° C. and 75%±5% RH.

In certain embodiments, the pharmaceutical composition disclosed herein maintains 100% of the amount of L-theanine after storage for 6 months at 40° C.±2° C. and 75%±5% RH.

In certain embodiments, the pharmaceutical composition disclosed herein provides a dissolution of at least 75% L-theanine at 60 minutes under USP <2040> conditions.

In certain embodiments, the pharmaceutical composition disclosed herein provides a dissolution of at least 85% L-theanine at 60 minutes under USP <2040> conditions.

In certain embodiments, the pharmaceutical composition disclosed herein provides a dissolution of at least 95% L-theanine at 60 minutes under USP <2040> conditions.

In certain embodiments, the pharmaceutical composition disclosed herein provides a dissolution of 100% L-theanine at 60 minutes under USP <2040> conditions.

In certain embodiments, the pharmaceutical composition disclosed herein provides a dissolution of at least 75% L-theanine at 60 minutes under USP <2040> conditions after storage for 1 month at 40° C.±2° C. and 75%±5% RH

In certain embodiments, the pharmaceutical composition disclosed herein provides a dissolution of at least 85% L-theanine at 60 minutes under USP <2040> conditions after storage for 1 month at 40° C.±2° C. and 75%±5% RH.

In certain embodiments, the pharmaceutical composition disclosed herein provides a dissolution of at least 95% L-theanine at 60 minutes under USP <2040> conditions after storage for 1 month at 40° C.±2° C. and 75%±5% RH.

In certain embodiments, the pharmaceutical composition disclosed herein provides a dissolution of 100% L-theanine at 60 minutes under USP <2040> conditions after storage for 1 month at 40° C.±2° C. and 75%±5% RH.

In certain embodiments, the pharmaceutical composition disclosed herein provides a dissolution of at least 75% L-theanine at 60 minutes under USP <2040> conditions after storage for 2 months at 40° C.±2° C. and 75%±5% RH

In certain embodiments, the pharmaceutical composition disclosed herein provides a dissolution of at least 85% L-theanine at 60 minutes under USP <2040> conditions after storage for 2 months at 40° C.±2° C. and 75%±5% RH.

In certain embodiments, the pharmaceutical composition disclosed herein provides a dissolution of at least 95% L-theanine at 60 minutes under USP <2040> conditions after storage for 2 months at 40° C.±2° C. and 75%±5% RH.

In certain embodiments, the pharmaceutical composition disclosed herein provides a dissolution of 100% L-theanine at 60 minutes under USP <2040> conditions after storage for 2 months at 40° C.±2° C. and 75%±5% RH

In certain embodiments, the pharmaceutical composition disclosed herein provides a dissolution of at least 75% L-theanine at 60 minutes under USP <2040> conditions after storage for 3 months at 40° C.±2° C. and 75%±5% RH

In certain embodiments, the pharmaceutical composition disclosed herein provides a dissolution of at least 85% L-theanine at 60 minutes under USP <2040> conditions after storage for 3 months at 40° C.±2° C. and 75%±5% RH.

In certain embodiments, the pharmaceutical composition disclosed herein provides a dissolution of at least 95% L-theanine at 60 minutes under USP <2040> conditions after storage for 3 months at 40° C.±2° C. and 75%±5% RH.

In certain embodiments, the pharmaceutical composition disclosed herein provides a dissolution of 100% L-theanine at 60 minutes under USP <2040> conditions after storage for 3 months at 40° C.±2° C. and 75%±5% RH

In certain embodiments, the pharmaceutical composition disclosed herein provides a dissolution of at least 75% L-theanine at 60 minutes under USP <2040> conditions after storage for 6 months at 40° C.±2° C. and 75%±5% RH

In certain embodiments, the pharmaceutical composition disclosed herein provides a dissolution of at least 85% L-theanine at 60 minutes under USP <2040> conditions after storage for 6 months at 40° C.±2° C. and 75%±5% RH.

In certain embodiments, the pharmaceutical composition disclosed herein provides a dissolution of at least 95% L-theanine at 60 minutes under USP <2040> conditions after storage for 6 months at 40° C.±2° C. and 75%±5% RH.

In certain embodiments, the pharmaceutical composition disclosed herein provides a dissolution of 100% L-theanine at 60 minutes under USP <2040> conditions after storage for 6 months at 40° C.±2° C. and 75%±5% RH.

In certain embodiments, the pharmaceutical composition disclosed herein comprises zinc in an amount from about 5 mg to about 100 mg, about 10 mg to about 50 mg, about 20 mg to about 30 mg or about 25 mg.

In certain embodiments, the pharmaceutical composition disclosed herein maintains at least 90% of the amount of zinc after storage for 1 month at 40° C.±2° C. and 75%±5% RH.

In certain embodiments, the pharmaceutical composition disclosed herein maintains at least 95% of the amount of zinc after storage for 1 month at 40° C.±2° C. and 75%±5% RH.

In certain embodiments, the pharmaceutical composition disclosed herein maintains at least 98% of the amount of zinc after storage for 1 month at 40° C.±2° C. and 75%±5% RH.

In certain embodiments, the pharmaceutical composition disclosed herein maintains 100% of the amount of zinc after storage for 1 month at 40° C.±2° C. and 75%±5% RH.

In certain embodiments, the pharmaceutical composition disclosed herein maintains at least 90% of the amount of zinc after storage for 2 months at 40° C.±2° C. and 75%±5% RH.

In certain embodiments, the pharmaceutical composition disclosed herein maintains at least 95% of the amount of zinc after storage for 2 months at 40° C.±2° C. and 75%±5% RH.

In certain embodiments, the pharmaceutical composition disclosed herein maintains at least 98% of the amount of zinc after storage for 2 months at 40° C.±2° C. and 75%±5% RH.

In certain embodiments, the pharmaceutical composition disclosed herein maintains 100% of the amount of zinc after storage for 2 months at 40° C.±2° C. and 75%±5% RH.

In certain embodiments, the pharmaceutical composition disclosed herein maintains at least 90% of the amount of zinc after storage for 3 months at 40° C.±2° C. and 75%±5% RH.

In certain embodiments, the pharmaceutical composition disclosed herein maintains at least 95% of the amount of zinc after storage for 3 months at 40° C.±2° C. and 75%±5% RH.

In certain embodiments, the pharmaceutical composition disclosed herein maintains at least 98% of the amount of zinc after storage for 3 months at 40° C.±2° C. and 75%±5% RH.

In certain embodiments, the pharmaceutical composition disclosed herein maintains 100% of the amount of zinc after storage for 3 months at 40° C.±2° C. and 75%±5% RH.

In certain embodiments, the pharmaceutical composition disclosed herein maintains at least 90% of the amount of zinc after storage for 6 months at 40° C.±2° C. and 75%±5% RH.

In certain embodiments, the pharmaceutical composition disclosed herein maintains at least 95% of the amount of zinc after storage for 6 months at 40° C.±2° C. and 75%±5% RH.

In certain embodiments, the pharmaceutical composition disclosed herein maintains at least 98% of the amount of zinc after storage for 6 months at 40° C.±2° C. and 75%±5% RH.

In certain embodiments, the pharmaceutical composition disclosed herein maintains 100% of the amount of zinc after storage for 6 months at 40° C.±2° C. and 75%±5% RH.

In certain embodiments, the pharmaceutical composition disclosed herein provides a dissolution of at least 75% zinc at 60 minutes under USP <2040> conditions.

In certain embodiments, the pharmaceutical composition disclosed herein provides a dissolution of at least 85% zinc at 60 minutes under USP <2040> conditions.

In certain embodiments, the pharmaceutical composition disclosed herein provides a dissolution of at least 95% zinc at 60 minutes under USP <2040> conditions.

In certain embodiments, the pharmaceutical composition disclosed herein provides a dissolution of 100% zinc at 60 minutes under USP <2040> conditions.

In certain embodiments, the pharmaceutical composition disclosed herein provides a dissolution of at least 75% zinc at 60 minutes under USP <2040> conditions after storage for 1 month at 40° C.±2° C. and 75%±5% RH

In certain embodiments, the pharmaceutical composition disclosed herein provides a dissolution of at least 85% zinc at 60 minutes under USP <2040> conditions after storage for 1 month at 40° C.±2° C. and 75%±5% RH.

In certain embodiments, the pharmaceutical composition disclosed herein provides a dissolution of at least 95% zinc at 60 minutes under USP <2040> conditions after storage for 1 month at 40° C.±2° C. and 75%±5% RH.

In certain embodiments, the pharmaceutical composition disclosed herein provides a dissolution of 100% zinc at 60 minutes under USP <2040> conditions after storage for 1 month at 40° C.±2° C. and 75%±5% RH.

In certain embodiments, the pharmaceutical composition disclosed herein provides a dissolution of at least 75% zinc at 60 minutes under USP <2040> conditions after storage for 2 months at 40° C.±2° C. and 75%±5% RH

In certain embodiments, the pharmaceutical composition disclosed herein provides a dissolution of at least 85% zinc at 60 minutes under USP <2040> conditions after storage for 2 months at 40° C.±2° C. and 75%±5% RH.

In certain embodiments, the pharmaceutical composition disclosed herein provides a dissolution of at least 95% zinc at 60 minutes under USP <2040> conditions after storage for 2 months at 40° C.±2° C. and 75%±5% RH.

In certain embodiments, the pharmaceutical composition disclosed herein provides a dissolution of 100% zinc at 60 minutes under USP <2040> conditions after storage for 2 months at 40° C.±2° C. and 75%±5% RH

In certain embodiments, the pharmaceutical composition disclosed herein provides a dissolution of at least 75% zinc at 60 minutes under USP <2040> conditions after storage for 3 months at 40° C.±2° C. and 75%±5% RH

In certain embodiments, the pharmaceutical composition disclosed herein provides a dissolution of at least 85% zinc at 60 minutes under USP <2040> conditions after storage for 3 months at 40° C.±2° C. and 75%±5% RH.

In certain embodiments, the pharmaceutical composition disclosed herein provides a dissolution of at least 95% zinc at 60 minutes under USP <2040> conditions after storage for 3 months at 40° C.±2° C. and 75%±5% RH.

In certain embodiments, the pharmaceutical composition disclosed herein provides a dissolution of 100% zinc at 60 minutes under USP <2040> conditions after storage for 3 months at 40° C.±2° C. and 75%±5% RH

In certain embodiments, the pharmaceutical composition disclosed herein provides a dissolution of at least 75% zinc at 60 minutes under USP <2040> conditions after storage for 6 months at 40° C.±2° C. and 75%±5% RH

In certain embodiments, the pharmaceutical composition disclosed herein provides a dissolution of at least 85% zinc at 60 minutes under USP <2040> conditions after storage for 6 months at 40° C.±2° C. and 75%±5% RH.

In certain embodiments, the pharmaceutical composition disclosed herein provides a dissolution of at least 95% zinc at 60 minutes under USP <2040> conditions after storage for 6 months at 40° C.±2° C. and 75%±5% RH.

In certain embodiments, the pharmaceutical composition disclosed herein provides a dissolution of 100% zinc at 60 minutes under USP <2040> conditions after storage for 6 months at 40° C.±2° C. and 75%±5% RH.

The pharmaceutical composition of claim 1 or 13, comprising vitamin D3 in an amount from about 0.001 mg to about 1 mg, about 0.005 mg to about 0.5 mg, about 0.001 mg to about 0.01 mg or about 0.05 mg.

In certain embodiments, the pharmaceutical composition disclosed herein maintains at least 90% of the amount of vitamin D3 after storage for 1 month at 40° C.±2° C. and 75%±5% RH.

In certain embodiments, the pharmaceutical composition disclosed herein maintains at least 95% of the amount of vitamin D3 after storage for 1 month at 40° C.±2° C. and 75%±5% RH.

In certain embodiments, the pharmaceutical composition disclosed herein maintains at least 98% of the amount of vitamin D3 after storage for 1 month at 40° C.±2° C. and 75%±5% RH.

In certain embodiments, the pharmaceutical composition disclosed herein maintains 100% of the amount of vitamin D3 after storage for 1 month at 40° C.±2° C. and 75%±5% RH.

In certain embodiments, the pharmaceutical composition disclosed herein maintains at least 90% of the amount of vitamin D3 after storage for 2 months at 40° C.±2° C. and 75%±5% RH.

In certain embodiments, the pharmaceutical composition disclosed herein maintains at least 95% of the amount of vitamin D3 after storage for 2 months at 40° C.±2° C. and 75%±5% RH.

In certain embodiments, the pharmaceutical composition disclosed herein maintains at least 98% of the amount of vitamin D3 after storage for 2 months at 40° C.±2° C. and 75%±5% RH.

In certain embodiments, the pharmaceutical composition disclosed herein maintains 100% of the amount of vitamin D3 after storage for 2 months at 40° C.±2° C. and 75%±5% RH.

In certain embodiments, the pharmaceutical composition disclosed herein maintains at least 90% of the amount of vitamin D3 after storage for 3 months at 40° C.±2° C. and 75%±5% RH.

In certain embodiments, the pharmaceutical composition disclosed herein maintains at least 95% of the amount of vitamin D3 after storage for 3 months at 40° C.±2° C. and 75%±5% RH.

In certain embodiments, the pharmaceutical composition disclosed herein maintains at least 98% of the amount of vitamin D3 after storage for 3 months at 40° C.±2° C. and 75%±5% RH.

In certain embodiments, the pharmaceutical composition disclosed herein maintains 100% of the amount of vitamin D3 after storage for 3 months at 40° C.±2° C. and 75%±5% RH.

In certain embodiments, the pharmaceutical composition disclosed herein maintains at least 90% of the amount of vitamin D3 after storage for 6 months at 40° C.±2° C. and 75%±5% RH.

In certain embodiments, the pharmaceutical composition disclosed herein maintains at least 95% of the amount of vitamin D3 after storage for 6 months at 40° C.±2° C. and 75%±5% RH.

In certain embodiments, the pharmaceutical composition disclosed herein maintains at least 98% of the amount of vitamin D3 after storage for 6 months at 40° C.±2° C. and 75%±5% RH.

In certain embodiments, the pharmaceutical composition disclosed herein maintains 100% of the amount of vitamin D3 after storage for 6 months at 40° C.±2° C. and 75%±5% RH.

All references disclosed herein are incorporated by reference for all purposes.

### EXAMPLES

### Example 1. Formulation Having Multiple Mechanisms of Action

### Current Formulation Selected

A selection of one or more nutritional ingredients are selected based on their targeted effects within one or more of the six process pathways, that provide additive or synergistic effects via their respective mechanisms of action. The dosage format can be a capsule with blended powder contained within. Optionally, it can be prepared in other commonly used formats to those skilled in the art (eg, tablet, caplet, soft gel, gummy, etc)

Optionally, the nutritional ingredients can be prepared into a kit, wherein, one compartment of a capsule in a capsule, muli-layer tablet or caplet, where one part of the kit contains the nutritional ingredients, and the other compartment, a pharmaceutical medicament or medicaments, commonly used in the treatment of a specific mood disorder (eg, SSRI/SSNRIs, Antipsychotics, Anti-anxiety medicaments, Attention deficit, Autism, OCD).

The following ingredients are selected based on all having targeted effects in all the six pathways

### Ingredients and Targets

1. Neurotransmitter synthesis and release
   (a) L-methylfolate (dose range 1 mg-75 mg, preferred dose 15 mg): This bioactive form of folate is crucial for the synthesis of monoamine neurotransmitters such as serotonin, dopamine, and norepinephrine. It acts as a cofactor in the conversion of homocysteine to methionine, which is a precursor for S-adenosylmethionine (SAMe), a key methyl donor in the synthesis of these neurotransmitters. By enhancing monoamine synthesis, L-methylfolate can potentially improve mood and reduce anxiety symptoms.
   (b) Vitamin D3; dose range 1000IU-100,000IU, preferred dose 2000IU): can influence the brain's dopamine system. It can enhance dopamine synthesis and release, and modulate dopamine transporter activity, which is crucial for maintaining dopamine levels in the synaptic cleft. Additionally, vitamin D3 supplementation has been associated with increased serotonin levels, which can help alleviate anxiety symptoms. 2000IU is typically the minimal clinically effective dose.
   (c) Zinc; dose range 15 mg-250 mg (dependent on zinc form), preferred dose 25 mg zinc bisglycinate): Zinc plays a role in modulating neurotransmitter systems, including the serotonergic, dopaminergic, and glutamatergic systems. It can enhance the function of GABA, an inhibitory neurotransmitter, and modulate NMDA receptors, which are involved in excitatory neurotransmission. Zinc deficiency can increase anxiety, and supplementation can help restore normal neurotransmitter function and reduce anxiety symptoms.
   (d) L-theanine; dose range 25 mg-500 mg, or 250 mg): This amino acid can modulate levels of serotonin, dopamine, and norepinephrine in various brain regions, including the prefrontal cortex, nucleus accumbens, and hippocampus. L-theanine can increase GABA levels and reduce glutamate levels, promoting relaxation and reducing anxiety. Its anxiolytic effects are likely mediated through these changes in neurotransmitter levels and activity.
2 Glutamate modulation
   (a) L-methylfolate: modulates glutamate & NMDA receptors and restores optimal glutathione levels, lowering glutamate levels.
   (b) Vitamin D3: modulates the enzyme that converts glutamate to GABA reducing glutamate levels and prevent excitotoxicity.
   (c) Zinc: can regulate glutamate and GABA receptor activation and inactivation.
   (d) L-theanine: modulates glutamate and GABA levels through several mechanisms.
3. Oxidative stress modulation
   (a) L-methylfolate: iIncreases nitric oxide production, enhancing antioxidant properties and reducing free radical production.
   (b) L-theanine: reduces ROS and boosts antioxidant enzymes to mitigate oxidative stress
   (c) Vitamin D3: modulates ROS signaling to help reduce the risk of oxidative stress
   (d) Zinc: regulates ROS scavenger enzymes to eliminate free radicals in the synapse
4. Inflammation modulation
   (a) L-methylfolate: lowers inflammation by regulating pro-inflammatory markers through one-carbon & methylation cycles.
   (b) Vitamin D3: modulates the immune system to reduce pro-inflammatory cytokine production
   (c) L-theanine: inhibits nuclear factor kappa B (NF-κB) pathway, thereby reducing expression of proinflammatory factors, cyclooxygenase-2 (COX-2), prostaglandin E2, inducible nitric oxide synthase, as well as NO
5. Neural communication and plasticity
   (a) L-methylfolate: improves neuroplasticity through normalization of monoamine production
   (b) Zinc Bisglycinate: fFunctions as a neuromodulator, regulating neurogenesis and communication through various membrane receptors (NMDA), ion channels and transporters
   (c) L-theanine: can influence production of BDNF, as well as NMDA receptors involved in neuroplasticity
   (d) Vitamin D3: is a potent regulator of the production of neurotrophic substances, such as BDNF, a neurotrophin (NT)-3, and nerve growth factor (NGF)
6. Stress response modulation
   (a) L-theanine: modulates the HPA axis, lowering serum cortisol and glutamate levels to reduce chronic stress and its impact on neurons
   (b) Vitamin D3: mitigates HPA axis-induced inflammation by regulating the immune system and pro-inflammatory cytokines

### Example 2: Formulation Having Multiple Mechanisms of Action

A selection of one or more nutritional ingredients are selected based on their targeted effects within one or more of the six process pathways, that provide additive or synergistic effects via their respective mechanisms of action. The dosage format can be a capsule with blended powder contained within. Optionally, it can be prepared in other commonly used formats to those skilled in the art (eg, tablet, caplet, soft gel, gummy, etc)

Optionally, the nutritional ingredients can be prepared into a kit, wherein, one compartment of a capsule in a capsule, muli-layer tablet or caplet, where one part of the kit contains the nutritional ingredients, and the other compartment, a pharmaceutical medicament or medicaments, commonly used in the treatment of a specific mood disorder (eg, SSRI/SSNRIs, Antipsychotics, Anti-anxiety medicaments, Attention deficit, Autism, OCD).

The following ingredients are selected based on all having targeted effects in all the six pathways
the following ingredients are selected based on all having targeted effects in all the six pathways

### Ingredients

1. Calcium L-Methylfolate; dose range, 1 mg-50 mg; or 10 mg
2. Folinic acid; dose range 1-50 mg; or 25 mg
3. Vitamin B6 (Pyridoxal-5-Phosphate); dose range 1 mg-100 mg, or 50 mg
4. Vitamin D3; dose range, 500 mcg-100,000IU, or 5000IU
5. Magnesium; dose range 1 mg-500 mg (dependent also on mg type (oxide, glycinate, citrate etc), e.g., 50 mg mg glycinate
6. Saffron (Crocus sativus); dose range, 5 mg-100 mg, or 28 mg)

### Example 3: Formulation Having Multiple Mechanisms of Action

A selection of one or more nutritional ingredients are selected based on their targeted effects within one or more of the six process pathways, that provide additive or synergistic effects via their respective mechanisms of action. The dosage format can be a capsule with blended powder contained within. Optionally, it can be prepared in other commonly used formats to those skilled in the art (eg, tablet, caplet, soft gel, gummy, etc)

Optionally, the nutritional ingredients can be prepared into a kit, wherein, one compartment of a capsule in a capsule, muli-layer tablet or caplet, where one part of the kit contains the nutritional ingredients, and the other compartment, a pharmaceutical medicament or medicaments, commonly used in the treatment of a specific mood disorder (eg, SSRI/SSNRIs, Antipsychotics, Anti-anxiety medicaments, Attention deficit, Autism, OCD).

The following ingredients are selected based on all having targeted effects in all the six pathways

### Ingredients

1. Sodium L-methylfolate; dose range, 1 mg-50 mg; or 10 mg
2. Folinic acid; dose range 1-50 mg; or 25 mg
3. Vitamin D3; dose range, 500 mcg-100,000IU, or 5000IU
4. Iron; dose range 1 mg-500 mg (dependent also on mg type (oxide, glycinate, citrate etc), or 50 mg mg glycinate
5. Glutathione; dose range 100 mg-1000 mg
6. Saffron (Crocus sativus); dose range, 5 mg-100 mg, or 28 mg)

### Example 3: Novel Formulation Having Multiple Mechanisms of Action

7. Calcium L-methylfolate; dose range, 1 mg-50 mg; or 10 mg
8. Vitamin D3; dose range, 500 mcg-100,000IU, or 5000IU
9. Acetyl-L-carnitine; dose range, 250-3000 mg, or 1000 mg
10. Zinc; dose range 1 mg-500 mg (dependent also on mg type (oxide, glycinate, citrate etc), e.g., 50 mg mg glycinate
11. L-theanine; dose range 25 mg-500 mg, or 250 mg)

### Example 4

Immediate-release capsules were manufactured that include four medical food active ingredients: L-methylfolate calcium, L-theanine, zinc bisglycinate, and vitamin D3.

The actives were dispensed and sieved along with silicon dioxide through a 20-mesh screen to de-lump materials. The screened blend was collected in a stainless-steel intermediate bulk container (IBC) and tumbled in a Matcon IBC Blender for 30 minutes at 12 RPM. Separately, magnesium stearate, NF was passed through a 20-mesh screen to de-agglomerate and then added to the IBC. The blend was tumbled for an additional 4 minutes at 12 RPM to achieve the final lubricated blend. The final blend was encapsulated in a size 00 capsule.

The final formulation per unit (capsule) is in Table 4A below:

TABLE 4A

| | | | Quantity |
|---|---|---|---|
| | | % | per unit |
| Ingredient | Label Claim (mg) | Overage | (mg) |
| L-Methylfolate Calcium, | 15.0 (L-Methylfolate | 15% | 17.250 |
| USP² | Calcium) | | |
| L-theanine Granulation | 250.0 | 7% | 267.500 |
| Vitamin D3 100,000 IU | 0.05 | 30% | 26.000 |
| Zinc Bisglycinate | 25.0 | 15% | 28.750 |
| Silicon Dioxide | N/A | 0% | 6.060 |
| Magnesium Stearate, | N/A | 0% | 6.060 |
| USP-NF | | | |

The vitamin D3 entry reflects the mass of a vitamin D3 material used to deliver the stated label claim with overage.

### Example 5: Stability Studies

Accelerated stability studies were performed on 6-count and 90-count bottles of dosage forms stored at 40° C./75% RH. Assay (% recovery) was monitored for L-theanine, L-methylfolate (LMF), vitamin D3, and zinc, and dissolution was evaluated for L-theanine, LMF, and zinc. The dosage forms were assessed at TO, 1 month, 2 months, and 3 months and presented in Table 5A below:

TABLE 5A

| | | L-theanine | LMF | D3 | Zinc |
|---|---|---|---|---|---|
| Count | Timepoint(months) | (%) | (%) | (%) | (%) |
| 6 | 0 | 111.2 | 108.6 | 132.0 | 109.5 |
| 6 | 1 | 109.6 | 113.1 | 126.5 | 110.0 |
| 6 | 2 | 108.5 | 116.0 | 139.6 | 110.9 |
| 6 | 3 | 111.3 | 109.0 | 120.4 | 108.8 |
| 90 | 0 | 111.2 | 108.6 | 132.0 | 109.5 |
| 90 | 1 | 109.7 | 109.0 | 144.6 | 108.8 |
| 90 | 2 | 110.4 | 113.3 | 134.7 | 114.8 |
| 90 | 3 | 106.6 | 120.8 | 132.7 | 113.2 |

Across all configurations, assays remained within the predefined acceptance range of 90.0%-165.0% of the labeled amount of vitamin D, 90.0%-125.0% of the labeled amount of zinc and 90.0%-150.0% for the labeled amount of L-theanine and L-methylfolate Ca²⁺.

### Example 6: Dissolution Studies

Dissolution testing was conducted on the immediate-release capsules for L-theanine, LMF, and zinc. Testing was performed on 6-count and 90-count bottles of dosage forms stored a TO, 1 month, 2 months, and 3 months under accelerated storage (40° C./75% RH). The dissolution testing was bed on USP <2040> as follows:

### Dissolution for L-Theanine and L-Methylfolate Ca²⁺

Apparatus 1 (Baskets).
Media: 900 mL of 0.05 M Citrate Buffer (pH 6.0±0.1) with Ascorbic Acid to stabilize at 37.0° C.
100 RPM
6 units tested individually and equal volumes pooled to test as sample solution (tested by HPLC)
Acceptance Criteria: not less than 75% of label claim at 60 minutes.

### Dissolution for Zinc

Apparatus 1 (Baskets)
Media: 900 mL 0.1 N hydrochloric acid at 37.0° C.
100 RPM
6 units tested individually and equal volumes pooled to test as sample solution (tested by ICP-OES)
Acceptance Criteria: not less than 75% of label claim at 60 minutes.

The dissolution results are summarized below in Table 6A.

TABLE 6A

| | Timepoint | L-theanine | LMF | Zinc |
|---|---|---|---|---|
| Count | (months) | (%) | (%) | (%) |
| 6 | 0 | 111.0 | 115.9 | 117.6 |
| 6 | 1 | 110.5 | 117.1 | 116.8 |
| 6 | 2 | 111.2 | 114.4 | 117.2 |
| 6 | 3 | 108.1 | 108.9 | 110.8 |
| 90 | 0 | 111.0 | 115.9 | 117.6 |
| 90 | 1 | 112.6 | 117.7 | 116.0 |
| 90 | 2 | 111.8 | 115.3 | 120.8 |
| 90 | 3 | 112.6 | 116.4 | 113.6 |

All dissolution results exceeded the acceptance criteria.

Additional Examples: Formulation having multiple mechanisms of action with Ingredients selected from any combination of one or more of the following groups of ingredients, known to have an effect on one or more of the six process pathways:

### Neurotransmitter (Monoamine) Modulators

Folic acid, Folinic acid, 5-methyltetrahydrofolate (D- and/or L-isomers; 5MTH), Calcium L-Methylfolate (LMF) Sodium L-methylfolate, Glucosamine L-methylfolate, vitamin B12 (methylcobalamin), Vitamin B12 (cyanocobalamin, methylcobalamin), magnesium, 5-hydroxytryptophan (5-HTP), S-adenosylmethionine (SAMe); amino acids Acetyl-L-carnitine (ALC or ALCAR), L-Carnitine (Levocarnitine), Propionyl-L-Carnitine (PLC), Isovaleryl-L-Carnitine, alpha-lipoic acid, N-acetylcysteine, and L-tryptophan, Zinc gluconate, Zinc Picolinate, Zinc Citrate, Zinc Acetate, Zinc Sulfate, Zinc Methionine (Zinc Monomethionine), Zinc Orotate, Zinc Oxide; Iron, Magnesium Oxide (MgO), Magnesium Picolinate (MgPic), Magnesium Citrate, Magnesium L-Threonate (MgLT), Magnesium Glycinate (Bisglycinate), Magnesium Sulfate (MgSO₄); St. John's Wort, Oregano extract, Curcumin, Ginseng (Panax ginseng), Rhodiola rosea, Saffron (Crocus sativus), Luteolin, Tilianin (from Agastache mexicana) and L-theanine.

### Oxidative Stress Modulators

Folic acid, Folinic acid, 5-methyltetrahydrofolate (D- and/or L-isomers; 5MTH), Calcium L-Methylfolate (LMF) Sodium L-methylfolate, Glucosamine L-methylfolate, Acetyl-L-carnitine (ALC or ALCAR), L-Carnitine (Levocarnitine), Propionyl-L-Carnitine (PLC), Isovaleryl-L-Carnitine, glutathione, coenzyme Q10, N-acetylcysteine, inositol, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Apple phenolic extracts, Cinnamic acid, Curcumin, Flower essential oil of Tagetes minuta, p-Coumaric acid, Tilapia skin peptides, Flower essential oil of Tagetes minuta, Melatonin, Carvedilol, Saffron extract, Luteolin-7-O-glucuronide, Rosmarinic acid, Celastrol, Thymoquinone and L-theanine.

### Anti-Inflammatory Modulators

Calcium L-Methylfolate (LMF) Sodium L-methylfolate, vitamin D3, Zinc, Omega-3 fatty acids (DHA/EPA), Ahi flower oil, alpha-lipoic acid (ALA), medium chain triglyceride (MCT) oil, Curcumin, Boswellia, Resveratrol and L-theanine.

### Glutamate—GABA Modulators

folic acid, Folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Magnesium Picolinate (MgPic), Magnesium Citrate, Magnesium L-Threonate (MgLT), Magnesium Glycinate (Bisglycinate), Magnesium Sulfate (MgSO4); gamma amino butyric acid (GABA), and L-theanine.

### Neurotrophic and Synaptic Plasticity Modulators

folic acid, Folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide; Magnesium Picolinate (MgPic), Magnesium Citrate, Magnesium, L-Threonate (MgLT), Magnesium Glycinate (Bisglycinate), Magnesium Sulfate (MgSO4); Vitamin D3, Curcumin, Ginseng (Panax ginseng) Bacopa monniera; Flavonoids extracts found in various fruits, vegetables, and teas, including, polyphenols Persimmon leaf extract, and Curcumin.

### HPA Stress and Anxiety Modulators:

L-theanine, valerian root, lemon balm, passionflower, magnolia extract, phaelladendron extract, Saffron extract, and ashwagandha, 1-methylfolate, folinic acid, vitamin D3, and Zinc. Preferably, the composition is comprised of, L-theanine which modulates the HPA axis, lowering serum cortisol and glutamate levels to reduce chronic stress and its impact on neurons, and Vitamin D3 known to mitigate HPA axis-induced inflammation by regulating the immune system and pro-inflammatory cytokines.

### Kit Examples:

Formulation having multiple mechanisms of action with Ingredients selected from any combination of one or more of the following groups of ingredients having an effect on one or more of the six process pathways, and one or more pharmaceutical ingredients, used in treatment of mood disorders

### Kit 1:

Delivered in one dosage form (e.g. capsule in capsule, multi-layer caplet, tablet or gummy

### Kit 2:

Delivered in two separate dosage forms (e.g., nutritional ingredients in one dosage form and one or more pharmaceutical drugs in separate dosage forms, packaged into the same secondary packaging component

### Kit 3:

Delivered with an external modulation device, known to help reduce symptoms of mood, for example, an autonomic nervous stimulation device, or a treatment such as transmagnetic stimulation, electrical stimulation and other digital stimulation devices

1. A pharmaceutical composition comprising a therapeutically effective amount of a compound to treat neurotransmitter signaling disturbance and a therapeutically effective amount of a compound to treat stress response.

2. The pharmaceutical composition of claim 1, further comprising a therapeutically effective amount of a compound to treat glutamate overreactivity.

3. The pharmaceutical composition of claim 1, further comprising a therapeutically effective amount of a compound to treat oxidative stress.

4. The pharmaceutical composition of claim 1, further comprising a therapeutically effective amount of a compound to treat neuroinflammation.

5. The pharmaceutical composition of claim 1, further comprising a therapeutically effective amount of a compound to treat neural communication and plasticity.

6. The pharmaceutical composition of claim 1, wherein the compound to treat neurotransmitter signaling disturbance is selected from folic acid, folinic acid, 5-methyltetrahydrofolate (D- and/or L-isomers; 5MTH), Calcium L-Methylfolate (LMF) Sodium L-methylfolate, Glucosamine L-methylfolate, vitamin B12 (methylcobalamin), Vitamin B12 (cyanocobalamin), magnesium, 5-hydroxytryptophan (5-HTP), S-adenosylmethionine (SAMe); amino acids Acetyl-L-carnitine (ALC or ALCAR), L-Carnitine (Levocarnitine), Propionyl-L-Carnitine (PLC), Isovaleryl-L-Carnitine, alpha-lipoic acid, N-acetylcysteine, and L-tryptophan, Zinc gluconate, Zinc Picolinate, Zinc Citrate, Zinc Acetate, Zinc Sulfate, Zinc Methionine (Zinc Monomethionine), Zinc Orotate, Zinc Oxide; Iron, Magnesium Oxide (MgO), Magnesium Picolinate (MgPic), Magnesium Citrate, Magnesium L-Threonate (MgLT), Magnesium Glycinate (Bisglycinate), Magnesium Sulfate (MgSO4); St. John's Wort, Oregano extract, Curcumin, Ginseng (Panax ginseng), Rhodiola rosea, Saffron (Crocus sativus), Luteolin, Tilianin (from Agastache mexicana), L-theanine, pharmaceutically acceptable salts thereof and combinations thereof.

7. The pharmaceutical composition of claim 6, wherein the compound to treat neurotransmitter signaling disturbance is L-methylfolate or a pharmaceutically acceptable salt thereof.

8. The pharmaceutical composition of claim 1, wherein the compound to treat stress response is selected from L-theanine, valerian root, lemon balm, passionflower, magnolia extract, phaelladendron extract, saffron extract, and ashwagandha, 1-methylfolate, folinic acid, vitamin D3, Zinc, pharmaceutically acceptable salts thereof and combinations thereof.

9. (canceled)

10. The pharmaceutical composition of claim 2, wherein the compound to treat glutamate overreactivity is selected from folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Magnesium Picolinate (MgPic), Magnesium Citrate, Magnesium L-Threonate (MgLT), Magnesium Glycinate (Bisglycinate), Magnesium Sulfate (MgSO₄); gamma amino butyric acid (GABA), L-theanine and combinations thereof.

11. (canceled)

12. The pharmaceutical composition of claim 3, wherein the compound to treat oxidative stress is selected from folic acid, folinic acid, 5-methyltetrahydrofolate (D- and/or L-isomers; 5MTH), Calcium L-Methylfolate (LMF) Sodium L-methylfolate, Glucosamine L-methylfolate, Acetyl-L-carnitine (ALC or ALCAR), L-Carnitine (Levocarnitine), Propionyl-L-Carnitine (PLC), Isovaleryl-L-Carnitine, glutathione, coenzyme Q10, N-acetylcysteine, inositol, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide, Apple phenolic extracts, Cinnamic acid, Curcumin, Flower essential oil of Tagetes minuta, p-Coumaric acid, Tilapia skin peptides, Flower essential oil of Tagetes minuta, Melatonin, Carvedilol, Saffron extract, Luteolin-7-O-glucuronide, Rosmarinic acid, Celastrol, Thymoquinone, L-theanine and combinations thereof.

13. The pharmaceutical composition of claim 12, wherein the compound to treat oxidative stress is selected from 1-methylfolate, vitamin D3, L-theanine, zinc, pharmaceutically acceptable salts thereof and combinations thereof.

14. The pharmaceutical composition of claim 4, wherein the compound to treat neuroinflammation is selected from Calcium L-Methylfolate (LMF) Sodium L-methylfolate, vitamin D3, Zinc, Omega-3 fatty acids (DHA/EPA), Ahi flower oil, alpha-lipoic acid (ALA), medium chain triglyceride (MCT) oil, Curcumin, Boswellia, Resveratrol, L-theanine and combinations thereof.

15. (canceled)

16. The pharmaceutical composition of claim 5, wherein the compound to treat neural communication and plasticity is selected from folic acid, folinic acid, 5-methyltetrahydrofolate (D- and/or L-isomers; 5MTH), Calcium L-Methylfolate (LMF) Sodium L-methylfolate, Vitamin D3, Zinc gluconate, Zinc Picolinate, Zinc Citrate, Zinc Acetate, Zinc Bisglycinate, Zinc Sulfate, Zinc Methionine (Zinc Monomethionine), Zinc Orotate, Zinc Oxide; Magnesium Picolinate (MgPic), Magnesium Citrate, Magnesium, L-Threonate (MgLT), Magnesium Glycinate (Bisglycinate), Magnesium Sulfate (MgSO4); Vitamin D3, Curcumin, Ginseng (Panax ginseng) Bacopa monniera; Flavonoids extracts found in various fruits, vegetables, and teas, including, polyphenols Persimmon leaf extract, and Curcumin.

17. (canceled)

18. The pharmaceutical composition of claim 5, comprising a therapeutically effective amount of a compound to treat neurotransmitter signaling disturbance, a therapeutically effective amount of a compound to treat stress response, a therapeutically effective amount of a compound to treat glutamate overreactivity, a therapeutically effective amount of a compound to treat oxidative stress, a therapeutically effective amount of a compound to treat neuroinflammation and a therapeutically effective amount of a compound to treat neural communication and plasticity.

19. The pharmaceutical composition of claim 18, wherein the therapeutically effective amount of a compound to treat neurotransmitter signaling disturbance comprises 1-methylfolate, the therapeutically effective amount of a compound to treat stress response comprises vitamin D3 and L-theanine, the therapeutically effective amount of a compound to treat glutamate overreactivity comprises 1-methylfolate and vitamin D3, the therapeutically effective amount of a compound to treat oxidative stress comprises 1-methylfolate, vitamin D3, L-theanine and zinc, the therapeutically effective amount of a compound to treat neuroinflammation comprises 1-methylfolate and vitamin D3 and the therapeutically effective amount of a compound to treat neural communication and plasticity comprises 1-methylfolate and zinc.

20. The pharmaceutical composition of claim 19 that is selected from a dosage form for oral, parenteral, subcutaneous, intramuscular, intraperitoneal, intravenous, transdermal, transmucosal, transurethral, rectal, sublingual, buccal, gingival, rectal, subcutaneous, transpulmonary, topical, nasal, or inhalation administration.

21. The pharmaceutical composition of claim 20 that is an oral dosage form.

22. The pharmaceutical composition of claim 21, wherein the oral dosage form is a tablet, capsule, softgel, liquid, suspension, solution or powder.

23. The pharmaceutical composition of claim 22, wherein the oral dosage form comprises from about 1 mg to about 75 mg 1-methylfolate, from about 1,000 IU to about 100,000 IU vitamin D3, from about 25 mg to about 500 mg L-theanine and from about 15 mg to about 250 mg zinc.

24-30. (canceled)

31. A method of treating mood disorders comprising administering to a patient in need thereof a therapeutically effective amount of a compound to treat neurotransmitter signaling disturbance and a therapeutically effective amount of a compound to treat stress response, wherein the compounds are in the same or different pharmaceutical compositions.

32-259. (canceled)
